(19) United States
(12) Patent Application Publication
(10) Pub. No.: US 2025/0277032 A1
(43) Pub. Date: Sep. 4, 2025

(54) ANTI-ILT3 ANTIBODIES AND USE THEREOF

(71) Applicant: BIOND BIOLOGICS LTD., Misgav Industrial Park (IL)

(72) Inventors: Tsuri PERETZ, Yokneam ilit (IL); Ilana MANDEL, Manof (IL); Yoav PIZEM, Alon Hagalil (IL); Liat IANCOVICI, Haifa (IL); Motti HAKIM, Kibbutz Gazit (IL); Avidor SHULMAN, Rakefet (IL); Yair SAPIR, Manof (IL); Tehila BEN-MOSHE, Tel Aviv (IL)

(21) Appl. No.: 18/858,887

(22) PCT Filed: Apr. 25, 2023
(86) PCT No.: PCT/IL2023/050426

§ 371 Date: Oct. 22, 2024

Related U.S. Application Data
(60) Provisional application No. 63/334,365, filed on Apr. 25, 2022.

(51) Int. Cl. C07K 16/28; A61K 39/00; A61P 35/00

(52) U.S. Cl. CPC C07K 16/2803; A61P 35/00; A61K 2039/505; C07K 2317/24; C07K 2317/33

(57) ABSTRACT

Antibodies that bind Leukocyte immunoglobulin-like receptor subfamily B member 4 (ILT3) are provided. Nucleic acid molecules encoding the antibodies, compositions comprising the antibodies and methods of using the antibodies are also provided.

### CROSS REFERENCE TO RELATED APPLICATIONS

This application claims the benefit of priority of U.S. Provisional Patent Application No. 63/334,365, filed Apr. 25, 2022, the contents of which is incorporated herein by reference in its entirety.

### REFERENCE TO AN ELECTRONIC SEQUENCE LISTING

The contents of the electronic sequence listing (BDB-P-018-PCT SQL.xml; Size: 89,038 bytes; and Date of Creation: Apr. 3, 2023) is herein incorporated by reference in its entirety.

### FIELD OF INVENTION

The present invention is in the field of immune checkpoint inhibition.

### BACKGROUND OF THE INVENTION

During cancer development, cancer cells can evade immune surveillance through various mechanisms. Understanding these mechanisms of escape is critical for developing novel cancer therapy. Cancer immunotherapy aims to re-activate the patients' immune system for the elimination of cancer cells and is currently a highly active area in cancer research with unprecedented results in the clinic. Despite these advances, there is a constant need for new therapies that will allow for a more substantial increase in patients' mortality and quality of life as a standalone treatment or in combination with other agents.

Leukocyte immunoglobulin-like receptor subfamily B member 4, (ILT3, CD85k, LILRB4) is expressed on various myeloid cells and specially on myeloid cells with immunosuppressive activity including tumor associated macrophages (TAM), myeloid derived suppressor cells (MDSCs) and tolerogenic dendritic cells (DCtol). ILT3 contains 2 extracellular immunoglobulin-like domains and 3 intracellular tyrosine-based inhibitory motifs (ITIMs). The expression of ILT3 in myeloid cells and in a soluble form was reported in various malignancies in association with tumor immune escape, metastasis and poor prognosis. ILT3 signaling on myeloid cells leads to reduced expression of co-stimulatory receptors and differentiation to an immunosuppressive phenotype. In addition to regulation of myeloid cell maturation and function, ILT3 signaling induces, through unknown ligands, T cell anergy, and differentiation to T regulatory and T suppressor phenotype.

For many years ILT3 ligands remained undefined, but recently there is accumulating evidence that ILT3 binds proteins in the tumor microenvironment (TME) and tumor extracellular matrix (ECM). Proteins such as Apolipoprotein E (APOE) and Fibronectin (FN1) are present in the TME and can bind ILT3. The binding of ILT3 to its ligands induces an immunosuppressive phenotype in myeloid cells, mediates the inhibition of T cells activity and creates and immunosuppressive TME which supports tumor growth and proliferation.

In concordance with this reported data, the inhibition of ILT3 using blocking antibodies is thought to increase tumor cell elimination. This may occur by inhibition of the immunosuppressive effect of tumor resident myeloid cells, thereby inducing proinflammatory phenotype of myeloid cells which support tumor infiltrated T cells' (TILs') activation and thereby remodeling the TME from immunosuppressive to proinflammatory TME. In total, ILT3 blocking increases the effector activity of various immune cells against the malignant cells. Anti-ILT3 antibodies have been disclosed in International Patent Applicants WO2006/138739, WO2018/089300, WO2019/099597, and WO2020/056077A however, new superior anti-ILT3 antibodies that target the multiple pathways by which ILT3 can create an immunosuppressive environment, and with a strong and lasting effect are still greatly needed.

### SUMMARY OF THE INVENTION

The present invention provides antibodies that bind Leukocyte immunoglobulin-like receptor subfamily B member 4 (ILT3). Nucleic acid molecules encoding the antibodies, compositions comprising the antibodies and methods of using the antibodies are also provided.

According to a first aspect, there is provided an antibody or antigen binding fragment thereof comprising three heavy chain CDRs (CDR-H) and three light chain CDRs (CDR-L), wherein:
CDR-H1 comprises the amino acid sequence set forth in SEQ ID NO: 1 (GYSFX₁GF) wherein X₁ is S or T, CDR-H2 comprises the amino acid sequence as set forth in SEQ ID NO: 2 (FPSX₂GE) wherein X₂ is S or N, CDR-H3 comprises the amino acid sequence as set forth in SEQ ID NO: 3 (QAFYYFDX₃) wherein X₃ is S or Y, CDR-L1 comprises the amino acid sequence as set forth in SEQ ID NO: 4 (KSSQSLLSSSNQKNYLA), CDR-L2 comprises the amino acid sequence as set forth in SEQ ID NO: 5 (WASTRES), and CDR-L3 comprises the amino acid sequence as set forth in SEQ ID NO: 6 (QQYYSYPLT).

According to another aspect, there is provided an antibody or antigen binding fragment thereof comprising three heavy chain CDRs (CDR-H) and three light chain CDRs (CDR-L), wherein:
CDR-H1 comprises the amino acid sequence set forth in SEQ ID NO: 49 (SYAMS), CDR-H2 comprises the amino acid sequence as set forth in SEQ ID NO: 50 (AITFGGGNTYYPDSVKG), CDR-H3 comprises the amino acid sequence as set forth in SEQ ID NO: 51 (HGDGNYDFYAMDY), CDR-L1 comprises the amino acid sequence as set forth in SEQ ID NO: 52 (KSSQSLLNSGNQKNYLT), CDR-L2 comprises the amino acid sequence as set forth in SEQ ID NO: 5 (WASTRES), and CDR-L3 comprises the amino acid sequence as set forth in SEQ ID NO: 53 (QNDYSYPLT).

According to another aspect, there is provided an antibody or antigen binding fragment thereof comprising three heavy chain CDRs (CDR-H) and three light chain CDRs (CDR-L), wherein:
CDR-H1 comprises the amino acid sequence set forth in SEQ ID NO: 49 (SYAMS), CDR-H2 comprises the amino acid sequence as set forth in SEQ ID NO: 56 (TISSDGGNTYYTDSVKG), CDR-H3 comprises the amino acid sequence as set forth in SEQ ID NO: 57 (HDGRGALDY), CDR-L1 comprises the amino acid sequence as set forth in SEQ ID NO: 58 (RASQDISNYLN), CDR-L2 comprises the amino acid sequence as set forth in SEQ ID NO: 59 (YTSRLHS), and CDR-L3 comprises the amino acid sequence as set forth in SEQ ID NO: 60 (QQGNTLPWT).

According to another aspect, there is provided an antibody or antigen binding fragment thereof comprising three heavy chain CDRs (CDR-H) and three light chain CDRs (CDR-L), wherein:
CDR-H1 comprises the amino acid sequence set forth in SEQ ID NO: 63 (NSAVH), CDR-H2 comprises the amino acid sequence as set forth in SEQ ID NO: 64 (VIWAGGNTNYNSTLMS), CDR-H3 comprises the amino acid sequence as set forth in SEQ ID NO: 65 (HETYGDSFDY), CDR-L1 comprises the amino acid sequence as set forth in SEQ ID NO: 66 (RSSQSLLDSDGKTYLN), CDR-L2 comprises the amino acid sequence as set forth in SEQ ID NO: 67 (LVSKLDS), and CDR-L3 comprises the amino acid sequence as set forth in SEQ ID NO: 68 (WQGTHFPFT).

According to another aspect, there is provided an antibody or antigen binding fragment thereof that competes with the antibody or antigen binding fragment of the invention for binding to ILT3.

According to another aspect, there is provided a nucleic acid molecule or a plurality of nucleic acid molecules encoding an antibody or antigen binding fragment of the invention.

According to another aspect, there is provided a pharmaceutical composition comprising an antibody or antigen binding fragment of the invention and a pharmaceutically acceptable carrier, excipient or adjuvant.

According to another aspect, there is provided a method of treating cancer in a subject in need thereof, the method comprising administering to the subject an antibody or antigen binding fragment thereof of the invention, thereby treating cancer.

According to another aspect, there is provided a method of treating cancer in a subject in need thereof, the method comprising administering to the subject a pharmaceutical composition of the invention, thereby treating cancer.

According to some embodiments,

a. SEQ ID NO: 1 is SEQ ID NO: 15 (GYSFTGF),
SEQ ID NO: 2 is SEQ ID NO: 16 (FPSNGE) and SEQ ID
NO: 3 is SEQ ID NO: 17 (QAFYYFDY);
b. SEQ ID NO: 1 is SEQ ID NO: 15 (GYSFTGF),
SEQ ID NO: 2 is SEQ ID NO: 16 (FPSNGE) and SEQ ID
NO: 3 is SEQ ID NO: 18 (QAFYYFDS);
c. SEQ ID NO: 1 is SEQ ID NO: 15 (GYSFTGF),
SEQ ID NO: 2 is SEQ ID NO: 19 (FPSSGE) and SEQ ID
NO: 3 is SEQ ID NO: 17 (QAFYYFDY);
d. SEQ ID NO: 1 is SEQ ID NO: 20 (GYSFSGF),
SEQ ID NO: 2 is SEQ ID NO: 16 (FPSNGE) and SEQ ID
NO: 3 is SEQ ID NO: 17 (QAFYYFDY);
e. SEQ ID NO: 1 is SEQ ID NO: 20 (GYSFSGF),
SEQ ID NO: 2 is SEQ ID NO: 19 (FPSSGE) and SEQ ID
NO: 3 is SEQ ID NO: 17 (QAFYYFDY);
or
f. SEQ ID NO: 1 is SEQ ID NO: 20 (GYSFSGF),
SEQ ID NO: 2 is SEQ ID NO: 19 (FPSSGE) and SEQ ID
NO: 3 is SEQ ID NO: 18 (QAFYYFDS).

According to some embodiments, the antibody or antigen binding fragment comprises at least one of:
a. a heavy chain variable region comprising the amino acid sequence of SEQ ID NO: 34 and a light chain variable region comprising the amino acid sequence of SEQ ID NO: 35;
b. a heavy chain variable region comprising the amino acid sequence of SEQ ID NO: 36 and a light chain variable region comprising the amino acid sequence of SEQ ID NO: 37;
c. a heavy chain variable region comprising the amino acid sequence of SEQ ID NO: 38 and a light chain variable region comprising the amino acid sequence of SEQ ID NO: 39;
d. a heavy chain variable region comprising the amino acid sequence of SEQ ID NO: 40 and a light chain variable region comprising the amino acid sequence of SEQ ID NO: 41;
e. a heavy chain variable region comprising the amino acid sequence of SEQ ID NO: 42 and a light chain variable region comprising the amino acid sequence of SEQ ID NO: 41;
f. a heavy chain variable region comprising the amino acid sequence of SEQ ID NO: 42 and a light chain variable region comprising the amino acid sequence of SEQ ID NO: 39;
g. a heavy chain variable region comprising the amino acid sequence of SEQ ID NO: 43 and a light chain variable region comprising the amino acid sequence of SEQ ID NO: 39;
h. a heavy chain variable region comprising the amino acid sequence of SEQ ID NO: 44 and a light chain variable region comprising the amino acid sequence of SEQ ID NO: 45;
i. a heavy chain variable region comprising the amino acid sequence of SEQ ID NO: 44 and a light chain variable region comprising the amino acid sequence of SEQ ID NO: 39;
j. a heavy chain variable region comprising the amino acid sequence of SEQ ID NO: 46 and a light chain variable region comprising the amino acid sequence of SEQ ID NO: 39;
k. a heavy chain variable region comprising the amino acid sequence of SEQ ID NO: 47 and a light chain variable region comprising the amino acid sequence of SEQ ID NO: 39; and
l. a heavy chain variable region comprising the amino acid sequence of SEQ ID NO: 48 and a light chain variable region comprising the amino acid sequence of SEQ ID NO: 39.

According to some embodiments, the antibody or antigen binding fragment comprises a heavy chain variable region comprising the amino acid sequence of SEQ ID NO: 54 and a light chain variable region comprising the amino acid sequence of SEQ ID NO: 55.

According to some embodiments, the antibody or antigen binding fragment comprises a heavy chain variable region comprising the amino acid sequence of SEQ ID NO: 61 and a light chain variable region comprising the amino acid sequence of SEQ ID NO: 62.

According to some embodiments, the antibody or antigen binding fragment comprises a heavy chain variable region comprising the amino acid sequence of SEQ ID NO: 69 and a light chain variable region comprising the amino acid sequence of SEQ ID NO: 70.

According to some embodiments, the heavy chain comprises an IgG4 constant region.

According to some embodiments, the IgG4 constant region comprises a sequence with at least 80% sequence identity to SEQ ID NO: 21

(ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRVE
SKYGPPCPPCPAPEFEGGPSVFLFSPKPKDTLMISRTPEVTCVVVDVSQE
DPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLGK).

According to some embodiments, the sequence with at least 80% sequence identity to SEQ ID NO: 21 comprises an S124P and an L131E mutation in SEQ ID NO: 21.

According to some embodiments, the light chain comprises a kappa constant region, optionally wherein the kappa constant region comprises a sequence with at least 80% sequence identity to SEQ ID NO: 22

(RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVT
KSFNRGEC).

According to some embodiments, the antibody of antigen binding fragment comprises at least one of
a. a heavy chain comprising the amino acid sequence of SEQ ID NO: 7 and a light chain comprising the amino acid sequence of SEQ ID NO: 8;
b. a heavy chain comprising the amino acid sequence of SEQ ID NO: 9 and a light chain comprising the amino acid sequence of SEQ ID NO: 10;
c. a heavy chain comprising the amino acid sequence of SEQ ID NO: 11 and a light chain comprising the amino acid sequence of SEQ ID NO: 12;
d. a heavy chain comprising the amino acid sequence of SEQ ID NO: 23 and a light chain comprising the amino acid sequence of SEQ ID NO: 24;
e. a heavy chain comprising the amino acid sequence of SEQ ID NO: 25 and a light chain comprising the amino acid sequence of SEQ ID NO: 24;
f. a heavy chain comprising the amino acid sequence of SEQ ID NO: 25 and a light chain comprising the amino acid sequence of SEQ ID NO: 12;
g. a heavy chain comprising the amino acid sequence of SEQ ID NO: 26 and a light chain comprising the amino acid sequence of SEQ ID NO: 12;
h. a heavy chain comprising the amino acid sequence of SEQ ID NO: 27 and a light chain comprising the amino acid sequence of SEQ ID NO: 12;
i. a heavy chain comprising the amino acid sequence of SEQ ID NO: 27 and a light chain comprising the amino acid sequence of SEQ ID NO: 28;
j. a heavy chain comprising the amino acid sequence of SEQ ID NO: 29 and a light chain comprising the amino acid sequence of SEQ ID NO: 12;
k. a heavy chain comprising the amino acid sequence of SEQ ID NO: 30 and a light chain comprising the amino acid sequence of SEQ ID NO: 12; and
l. a heavy chain comprising the amino acid sequence of SEQ ID NO: 31 and a light chain comprising the amino acid sequence of SEQ ID NO: 12.

According to some embodiments, the antibody or antigen binding fragment is capable of binding Leukocyte immunoglobulin-like receptor subfamily B member 4 (LILRB4 or ILT3).

According to some embodiments, ILT3 comprises the amino acid sequence provided in SEQ ID NO: 71.

According to some embodiments, the ILT3 is on the surface of myeloid cells, dendritic cells, macrophages or a combination thereof.

According to some embodiments, the myeloid cells are myeloid derived suppressor cells (MDSC), the dendritic cells are tolerogenic dendritic cells, the macrophages are suppressive macrophages, optionally wherein the macrophages are tumor associated macrophages (TAMs) or a combination thereof.

According to some embodiments, the binding to ILT3 inhibits binding of ILT3 to an ILT3 ligand.

According to some embodiments, the binding inhibits binding of ILT3 to both Apolipoprotein E (APOE) and fibronectin (FN1).

According to some embodiments, the APOE, FN1 or both are present in a tumor microenvironment (TME).

According to some embodiments, the binding to ILT3 releases T cells, monocytes, macrophages or a combination thereof from ILT3-mediated inhibition.

According to some embodiments, release of the T cells comprises increased T cell proliferation, increased pro-inflammatory cytokine secretion by the T cells, increased pro-inflammatory chemokine secretion by said T cells or a combination thereof.

According to some embodiments, the binding to ILT3 increases dendritic cell activation.

According to some embodiments, increasing dendritic cell activation comprises restoring dendritic cell activation suppressed by FN1, APOE or both.

According to some embodiments, increasing dendritic cell activation comprises increasing secretion of a pro-inflammatory cytokine or chemokine by the dendritic cells.

According to some embodiments, the binding to ILT3 increases monocyte and/or macrophage activation.

According to some embodiments, increasing monocyte and/or macrophage activation comprises increasing secretion of a pro-inflammatory cytokine or chemokine by the dendritic cells.

According to some embodiments, the pro-inflammatory cytokine is selected from interferon gamma (IFNG) interleukin 8 (IL-8) and tumor necrosis factor alpha (TNF-α), said pro-inflammatory chemokine is selected from C—C Motif Chemokine Ligand 3 (CCL3) and CCLA, or both.

According to some embodiments, the antigen binding fragment is selected from the group consisting of a Fv, Fab, F(ab′)2, scFV or a scFV2 fragment.

According to some embodiments, the antibody or antigen binding fragment is humanized.

According to some embodiments, the antibody is a monoclonal antibody.

According to some embodiments, the agent does not induce antibody-dependent cell-mediated cytotoxicity (ADCC) or complement-dependent cytotoxicity (CDC).

According to some embodiments, the pharmaceutical composition is formulated for administration to a subject.

According to some embodiments, the pharmaceutical composition is formulated for systemic administration or intratumoral administration.

According to some embodiments, the cancer is a solid cancer.

According to some embodiments, the cancer is selected from breast cancer, kidney cancer, head and neck cancer, lung cancer, sarcoma, gastric cancer, colorectal cancer and ovarian cancer.

According to some embodiments, the cancer is characterized by the presence of tumor infiltrating immune cells expressing ILT3.

According to some embodiments, the cancer TME is characterized by expression of APOE, FN1 or both.

According to some embodiments, expression is overexpression as compared to non-cancerous tissue of the same type as the tumor.

According to some embodiments, the method further comprises administering an immune checkpoint inhibitor (ICI).

According to some embodiments, the ICI inhibits the PD-1/PD-L1/L2 checkpoint.

According to some embodiments, the ICI is selected from pembrolizumab, nivolumab, atezolizumab, cemiplimab, dostarlimab, durvalumab and avelumab.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C: Histograms of ILT3 surface expression in (1A) monocytes and MDSCs derived from two healthy donors, (1B) immature dendritic cells (iDC), mature dendritic cells (mDC) and tolerogenic dendritic cells (DCtol) from two healthy donors, and (1C) monocytes that were differentiated into M0, M1 and M2 macrophages from two healthy donors. IC—isotype control, Mix—including anti-ILT3 antibody. Black lines show control cells incubated with an isotype control antibody and grey lines show anti-ILT3 PE-conjugated antibody staining of cells.

FIGS. 2A-2B: (2A) Micrographs of IHC staining of tissue samples for ILT3 (Red) and CD68 (Blue). Arrows indicate double-positive cells. (2B) Bar graph quantifying the number percent of all cancers sampled, for each cancer type, that were ILT3 positive.

FIGS. 3A-3B: (3A) A dot plot tracking relative MDSC enrichment of cancers from the TCGA database divided into 3 bins: low, medium and high ILT3 expression. (3B) Bar chart of M2 fraction enrichment relative to patients with different ILT3 levels in the same binned cancers.

FIGS. 4A-4B: (4A) Histograms of exogenous hILT3 expression on the surface of HEK cells as measured with a commercial antibody (ZM4.1) and the antibodies of the invention. Isotype control histograms are shown in black and ILT3 binding is shown in grey. (4B) Histograms of antibody (50 μg/ml) binding to cynomolgus ILT3. Isotype control histograms are shown in grey and ILT3 binding is shown in black.

FIG. 5: Line graph of ELISA detection of ILT3 when competing with a naked ZM4.1 antibody. ZM4.1 and antibody AB #2 compete for binding to ILT3, whereas 5E5 and AB #3 bind ILT3 at an epitope that results in no competition with ZM4.1

FIGS. 6A-6G: Bar graphs of (6A) TNFa secretion from PBMCs, (6B-6C) IFNg secretion from CD8 T cells in the presence of autologous MDSCs, (6D-6F) IFNg secretion from CD4 T cells in the presence of different dendritic cells in the presence or the absence of the 5E5 antibody, (6G) IFNg secretion from CD4 T cells in the presence of M2 macrophages produced in the presence or the absence of 5E5 antibody, and (6C) as compared to Merck's m52B8 antibody. (6E-6F) Combination of the 5E5 antibody with anti-PD-1 antibody is measured (6E) with anti-CD3 stimulation of the T cells and (6F) without the stimulation. A single donor is shown in 6A-6D though 3-4 donors were examined, and all showed comparable results. All donors are shown in 6E-6F and their individual increases are shown in the line graphs at the right. * P<0.05; ** P<0.01; un-paired Student's T-test compared to mIgG1.

FIGS. 7A-7E: Line graphs of blocking of ILT3 binding to (7A, 7C-7D) fibronectin and (7B, 7E) APOE in the presence of the 5E5 antibody and (7A-7B, 7D) the ZM4.1 antibody or (7C, 7E) the m52B8 antibody or (7D) other antibodies of the invention.

FIGS. 8A-8D: Bar graphs of (8A) IL-8 secretion from THP-1 cells, (8B) TNFa secretion from DCs, (8C) IL-8 secretion from DCs after activation with Rituximab and (8D) TNFa secretion from DCs after activation with Erbitux and culture with fibronectin with or without various anti-ILT3 antibodies. h52B8 is a humanized Merck anti-ILT3 antibody, IO202 is a humanized Immune-One Therapeutics anti-ILT3 antibody and h5A7 is a humanized NGM Bio anti-ILT3 antibody.

FIGS. 9A-9B: Bar graphs of TNFa secretion from (9A) ILT3 expressing DCs and (9B) non-expressing DCs cultured in the presence of fibronectin with and without 5E5 antibody. * P<0.05; un-paired Student's T-test compared to IgG.

FIG. 10: Line graph of binding of various h5E5 antibodies to hILT3. The EC₅₀ levels were determined using GraphPad software.

FIGS. 11A-11B: Line graphs of % blocking of ILT3 binding to FN1 by (11A) various h5E5 antibodies and (11B) a direct comparison of c5E5 and h5E5-133. The IC₅₀ levels were determined using GraphPad software.

FIGS. 12A-12D: Bar graphs of (12A) IFNg secretion from CD8 T cells in the presence of autologous MDSCs, (12B) TNFa secretion from DCs and (12C-12D) IL-8 secretion from THP-1 cells in the presence of fibronectin and Rituximab treated with (12C) c5E5 or h5E5, or (12D) m5E5 or alternative antibodies of the invention. 12A includes an inset of the 11 donor samples tested showing levels after administration of the isotype control and h5E5 at a concentration of 16 μg/ml.

FIGS. 13A-13B: Bar graph of TNFa secretion from monocytes differentiated into M1 activated macrophages (MF) in the presence of (13A) gastric cancer cells or (13B) colon cancer cells with and without the h5E5 antibody. * P<0.05; un-paired Student's T-test.

FIG. 14: Bar graphs of TNFa secretion from immature DCs stimulated with IL-10 and LPS to generate DCtols when cultured with or without anti-ILT3 antibodies or isotype control. Samples from 2 donors are shown.

FIGS. 15A-15B: Bar graphs of (15A) IFNg secretion from CD8 T cells incubated with autologous MDSC, and (15B) IFNg secretion from CD4 T cells co-cultured with the indicated dendritic cells treated with anti-PD1 antibody, h5E5 or a combination of the two.

FIGS. 16A-16D: (16A-16B) Bar graphs of IFNg production from (16A) sarcoma tumoroid and (16B) breast cancer tumoroid activated in the presence of h5E5 or isotype control. (16C-16D) Bar graphs of CCL3, CCL4 and IL-8 production from (16C) breast cancer tumoroids and (16D) CRC tumoroids activated in the presence of various anti-ILT3 antibodies, isotype control, pembrolizumab or combinations thereof. * P<0.05; ** P<0.01; *** P<0.005; un-paired Student's T-test.

FIG. 17: Line graph of MC38 tumor size over time in syngeneic transgenic hILT3 mice treated with c5E5 or an isotype control.

### DETAILED DESCRIPTION OF THE INVENTION

The present invention, in some embodiments, provides antibodies that bind Leukocyte immunoglobulin-like receptor subfamily B member 4 (ILT3). Nucleic acid molecules encoding the antibodies, compositions comprising the antibodies and methods of using the antibodies are also provided.

The invention is based on the generation of several ILT3 blocking antibodies using hybridoma technology. The lead antibody 5E5 demonstrated the requested binding profile, selectivity, cross-reactivity, blocking activity and activity in functional assays. This antibody was humanized using germline humanization. Humanized 5E5 is able to block ILT3 interaction with various ligands (e.g., APOE and FN1) and inhibit the immunosuppressive effect mediated by ILT3 signaling. By blocking and neutralizing ILT3, 5E5 remodulate the immunosuppressive TME, inducing pro-inflammatory phenotypes in tumor resident myeloid cells which results in enhanced tumor infiltrated T cell activation and tumor growth inhibition.

By a first aspect, there is provided an antibody or antigen binding fragment thereof that binds ILT3.

Leukocyte immunoglobulin-like receptor subfamily B member 4 is known as LILRB4, ILT3 and CD85K among many other names. The Entrez gene identifier for human ILT3 is 11006 and the mouse identifier is 14727. The protein sequence for human ILT3 can be found under the Uniprot identifier Q8NHJ6 and the mouse protein sequence under identifier Q61450. The human mRNA sequence can be found in RefSeq sequences NM_001081438, NM_001278426, NM_001278427, NM_001278428 and NM_001278429. The mouse mRNA sequence can be found in RefSeq sequences NM_001291892, NM_001291893 and NM_008147. The human protein sequence can be found in RefSeq sequences NP_001265355, NP_001265356, NP_001265357, NP_001265358 and NP_001265359. The mouse protein sequence can be found in RefSeq sequences NP001278821, NP_00127882 and NP_032173. In some embodiments, the amino acid sequence of ILT3 comprises MIPTFTALLCLGLSLGPRTHMQAGPLPKPTLWAEPGSVISWGNSVTIWCQGTLEAR EYRLDKEESPAPWDRQNPLEPKNKARFSIPSMTEDYAGRYRCYYRSPVGWSQPSD PLELVMTGAYSKPTLSALPSPLVTSGKSVTLLCQSRSPMDTFLLIKERAAHPLLHLR SEHGAQQHQAEFPMSPVTSVHGGTYRCFSSHGFSHYLLSHPSDPLELIVSGSLEDPR PSPTRSVSTAAGPEDQPLMPTGSVPHSGLRRHWE (SEQ ID NO: 71). In some embodiments, the amino acid sequence of ILT3 consists of SEQ ID NO: 71. In some embodiments, SEQ ID NO: 71 is human ILT3.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass.

In some embodiments, the antibody is a murine antibody. In some embodiments, the antibody is a chimeric antibody. In some embodiments, the antibody is a humanized antibody. In some embodiments, the antibody is a humanized antibody. In some embodiments, the antibody is a full IgG. In some embodiments, the antibody is an IgG2. In some embodiments, the antibody is an IgG4. In some embodiments, the antibody or antigen binding domain is an antigen binding domain that lacks an Fc domain.

In some embodiments, the peptide is selected from an antibody, an antigen binding fragment of an antibody, a Fab fragment, a single chain antibody, a single-domain antibody, a nanobody, a VHH antibody and an antibody mimetic. As used herein, the term “antibody mimetic” refers to an organic compound that can specifically bind to a target antigen. In some embodiments, an antibody mimetic is not structurally related to an antibody. Examples of antibody mimetics include, but are not limited to, affilins, affimers, affitins, alphabodies, anticalins, avimers, DARPins, fynomers, Kunitz domain peptides, monobodies, and nanoCLAMPS. In some embodiments, the antibody mimetic is a DARPin. All of these agents are well known in the art and are known to be useful in blocking interactions between receptors and their ligands. Small molecules and proteins that can bind mCD28 may occlude the cleavage site or may cause hinderance or impair access for the protease. In some embodiments, the protein is an antibody mimetic. As used herein, the term “DARPin” refers to a designed ankyrin repeat protein. DARPins are genetically engineered antibody mimetic proteins that are generally highly specific for their protein target. Thus, a DARPin for CD28 may be an example of an agent.

The basic unit of the naturally occurring antibody structure is a heterotetrameric glycoprotein complex of about 150,000 Daltons, composed of two identical light (L) chains and two identical heavy (H) chains, linked together by both noncovalent associations and by disulfide bonds. Each heavy and light chain also has regularly spaced intra-chain disulfide bridges. Five human antibody classes (IgG, IgA, IgM, IgD and IgE) exist, and within these classes, various subclasses, are recognized based on structural differences, such as the number of immunoglobulin units in a single antibody molecule, the disulfide bridge structure of the individual units, and differences in chain length and sequence. The class and subclass of an antibody is its isotype.

The amino terminal regions of the heavy and light chains are more diverse in sequence than the carboxy terminal regions, and hence are termed the variable domains. This part of the antibody structure confers the antigen-binding specificity of the antibody. A heavy variable (VH) domain and a light variable (VL) domain together form a single antigen-binding site, thus, the basic immunoglobulin unit has two antigen-binding sites. Particular amino acid residues are believed to form an interface between the light and heavy chain variable domains (Chothia et al., J. Mol. Biol. 186, 651-63 (1985); Novotny and Haber, (1985) Proc. Natl. Acad. Sci. USA 82 4592-4596).

The carboxy terminal portion of the heavy and light chains form the constant domains i.e. CH1, CH2, CH3, CL. While there is much less diversity in these domains, there are differences from one animal species to another, and further, within the same individual there are several different isotypes of antibody, each having a different function.

The term “framework region” or “FR” refers to the amino acid residues in the variable domain of an antibody, which are other than the hypervariable region amino acid residues as herein defined. The term “hypervariable region” as used herein refers to the amino acid residues in the variable domain of an antibody, which are responsible for antigen binding. The hypervariable region comprises amino acid residues from a “complementarity determining region” or “CDR”. The CDRs are primarily responsible for binding to an epitope of an antigen. The extent of FRs and CDRs has been precisely defined (see, Kabat et al.).

Immunoglobulin variable domains can also be analyzed using the IMGT information system (www://imgt.cines.fr/) (IMGTR/V-Quest) to identify variable region segments, including CDRs. See, e.g., Brochet, X. et al, Nucl. Acids Res. J6:W503-508 (2008).

Chothia et al. also defined a numbering system for variable domain sequences that is applicable to any antibody. One of ordinary skill in the art can unambiguously assign this system of “Chothia numbering” to any variable domain sequence, without reliance on any experimental data beyond the sequence itself. As used herein, “Chothia numbering” refers to the numbering system set forth by Chothia et al., Journal of Molecular Biology, “Canonical Structures for the Hypervariable regions of immunoglobulins” (1987) and Chothia et al., Nature, “Conformations of Immunoglobulin Hypervariable Regions” (1989).

As used herein, the term “humanized antibody” refers to an antibody from a non-human species whose protein sequences have been modified to increase similarity to human antibodies. A humanized antibody may be produced by production of recombinant DNA coding for the CDRs of the non-human antibody surrounded by sequences that resemble a human antibody. In some embodiments, the humanized antibody is a chimeric antibody. In some embodiments, humanizing comprises insertion of the CDRs of the invention into a human antibody scaffold or backbone. Humanized antibodies are well known in the art and any method of producing them that retains the CDRs of the invention may be employed.

In some embodiments, the antibody is a monoclonal antibody. The term “monoclonal antibody” or “mAb” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies and is not to be construed as produced by any specific preparation method. Monoclonal antibodies to be used in accordance with the methods provided herein, may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al, J. Mol. Biol. 222:581-597 (1991), for example.

The mAb of the present invention may be of any immunoglobulin class including IgG, IgM, IgD, IgE or IgA. A hybridoma producing a mAb may be cultivated in vitro or in vivo. High titers of mAbs can be obtained in vivo production where cells from the individual hybridomas are injected intraperitoneally into pristine-primed Balb/c mice to produce ascites fluid containing high concentrations of the desired mAbs. mAbs of isotype IgM or IgG may be purified from such ascites fluids, or from culture supernatants, using column chromatography methods well known to those of skill in the art.

“Antibody fragments” comprise a portion of an intact antibody, preferably comprising the antigen binding region thereof. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al, Protein Eng. 8(10): 1057-1062 (1995)); one-armed antibodies, single variable domain antibodies, minibodies, single-chain antibody molecules; multispecific antibodies formed from antibody fragments (e.g., including but not limited to, Db-Fc, taDb-Fc, taDb-CH3, (scFV) 4-Fc, di-scFv, bi-scFv, or tandem (di,tri)-scFv); and Bi-specific T-cell engagers (BiTEs).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three surfaces of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called a, delta, e, gamma, and micro, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. In some embodiments, the Fv polypeptide further comprises a polypeptide linker between the VH and VL domains that enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies production is known in the art and is described in Natl. Acad. Sci. USA, 90:6444-6448 (1993).

The term “multispecific antibody” is used in the broadest sense and specifically covers an antibody that has polyepitopic specificity. Such multispecific antibodies include, but are not limited to, an antibody comprising a heavy chain variable domain (VH) and a light chain variable domain (VL), where the VHVL unit has polyepitopic specificity, antibodies having two or more VL and VH domains with each VHVL unit binding to a different epitope, antibodies having two or more single variable domains with each single variable domain binding to a different epitope, full length antibodies, antibody fragments such as Fab, Fv, dsFv, scFv, diabodies, bispecific diabodies, triabodies, tri-functional antibodies, antibody fragments that have been linked covalently or non-covalently. “Polyepitopic specificity” refers to the ability to specifically bind to two or more different epitopes on the same or different target(s).

The monoclonal antibodies of the invention may be prepared using methods well known in the art. Examples include various techniques, such as those in Kohler, G. and Milstein, C, Nature 256:495-497 (1975); Kozbor et al, Immunology Today 4:72 (1983); Cole et al, pg. 77-96 in MONOCLONAL ANTIBODIES AND CANCER THERAPY, Alan R. Liss, Inc. (1985).

Besides the conventional method of raising antibodies in vivo, antibodies can be generated in vitro using phage display technology. Such a production of recombinant antibodies is much faster compared to conventional antibody production, and they can be generated against an enormous number of antigens. Furthermore, when using the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies, one can use various methods all based on display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

Non-human antibodies may be humanized by any methods known in the art. In one method, the non-human complementarity determining regions (CDRs) are inserted into a human antibody or consensus antibody framework sequence. Further changes can then be introduced into the antibody framework to modulate affinity or immunogenicity.

In some embodiments, antibodies and portions thereof include: antibodies, fragments of antibodies, Fab and F(ab′)2, single-domain antigen-binding recombinant fragments and natural nanobodies. In some embodiments, the antigen binding fragment is selected from the group consisting of a Fv, Fab, F(ab′)₂, scFV or a scFV2 fragment.

In some embodiments, the present invention provides nucleic acid sequences encoding the antibodies or antigen binding portions of the present invention.

For example, the polynucleotide may encode an entire immunoglobulin molecule chain, such as a light chain or a heavy chain. A complete heavy chain includes not only a heavy chain variable region (VH) but also a heavy chain constant region (CH), which typically will comprise three constant domains: CH1, CH2 and CH3; and a “hinge” region. In some situations, the presence of a constant region is desirable.

Other polypeptides which may be encoded by the polynucleotide include antigen-binding antibody fragments such as single domain antibodies (“dAbs”), Fv, scFv, Fab′ and CHI and CK or CL domain has been excised. As minibodies are smaller than conventional antibodies they should achieve better tissue penetration in clinical/diagnostic use but being bivalent they should retain higher binding affinity than monovalent antibody fragments, such as dAbs. Accordingly, unless the context dictates otherwise, the term “antibody” as used herein encompasses not only whole antibody molecules, but also antigen-binding antibody fragments of the type discussed above. Each framework region present in the encoded polypeptide may comprise at least one amino acid substitution relative to the corresponding human acceptor framework. Thus, for example, the framework regions may comprise, in total, three, four, five, six, seven, eight, nine, ten, eleven, twelve, thirteen, fourteen, or fifteen amino acid substitutions relative to the acceptor framework regions. Given the properties of the individual amino acids comprising the disclosed protein products, some rational substitutions will be recognized by the skilled worker. Amino acid substitutions, i.e. “conservative substitutions,” may be made, for instance, on the basis of similarity in polarity, charge, solubility, hydrophobicity, hydrophilicity, and/or the amphipathic nature of the residues involved.

Suitably, the polynucleotides described herein may be isolated and/or purified. In some embodiments, the polynucleotides are isolated polynucleotides.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring”.

In some embodiments, the antibody or antigen-binding portion thereof, comprising three heavy chain CDRs (CDR-H) and three light chain CDRs (CDR-L), wherein: CDR-H1 comprises the amino acid sequence set forth in SEQ ID NO: 1 (GYSFX₁GF) wherein X₁ is S or T, CDR-H2 comprises the amino acid sequence as set forth in SEQ ID NO: 2 (FPSX₂GE) wherein X₂ is S or N, CDR-H3 comprises the amino acid sequence as set forth in SEQ ID NO: 3 (QAFYYFDX₃) wherein X₃ is S or Y, CDR-L1 comprises the amino acid sequence as set forth in SEQ ID NO: 4 (KSSQSLLSSSNQKNYLA), CDR-L2 comprises the amino acid sequence as set forth in SEQ ID NO: 5 (WASTRES), and CDR-L3 comprises the amino acid sequence as set forth in SEQ ID NO: 6 (QQYYSYPLT). In some embodiments, the CDRs are numbered by the Chothia numbering system and the CDRs comprise SEQ ID NO: 1-6.

In some embodiments, the antibody or antigen-binding portion thereof, comprising three heavy chain CDRs (CDR-H) and three light chain CDRs (CDR-L), wherein: CDR-H1 comprises the amino acid sequence set forth in SEQ ID NO: 13 (GFYID), CDR-H2 comprises the amino acid sequence as set forth in SEQ ID NO: 14 (YIFPSX₂GETSYNQKFKG) wherein X₂ is S or N, CDR-H3 comprises the amino acid sequence as set forth in SEQ ID NO: 3 (QAFYYFDX₃) wherein X₃ is S or Y, CDR-L1 comprises the amino acid sequence as set forth in SEQ ID NO: 4 (KSSQSLLSSSNQKNYLA), CDR-L2 comprises the amino acid sequence as set forth in SEQ ID NO: 5 (WASTRES), and CDR-L3 comprises the amino acid sequence as set forth in SEQ ID NO: 6 (QQYYSYPLT). In some embodiments, the CDRs are numbered by the Kabat numbering system and the CDRs comprise SEQ ID NO: 13-14 and 3-6. It will be understood by a skilled artisan that the Chothia and Kabat systems are interchangeable and that CDRs given for either system can be converted into the CDRs of the other system.

In some embodiments, SEQ ID NO: 1 is SEQ ID NO: 15 (GYSFTGF). In some embodiments, SEQ ID NO: 1 is SEQ ID NO: 20 (GYSFSGF). In some embodiments, SEQ ID NO: 2 is SEQ ID NO: 16 (FPSNGE). In some embodiments, SEQ ID NO: 2 is SEQ ID NO: 19 (FPSSGE). In some embodiments, SEQ ID NO: 3 is SEQ ID NO: 17 (QAFYYFDY). In some embodiments, SEQ ID NO: 3 is SEQ ID NO: 18 (QAFYYFDS). In some embodiments, SEQ ID NO: 1 is SEQ ID NO: 15, SEQ ID NO: 2 is SEQ ID NO: 16 and SEQ ID NO: 3 is SEQ ID NO: 17. In some embodiments, SEQ ID NO: 3 is SEQ ID NO: 18 (QAFYYFDS). In some embodiments, SEQ ID NO: 14 is SEQ ID NO: 32 (YIFPSNGETSYNQKFKG). In some embodiments, SEQ ID NO: 14 is SEQ ID NO: 33 (YIFPSSGETSYNQKFKG).

In some embodiments, SEQ ID NO: 1 is SEQ ID NO: 15, SEQ ID NO: 2 is SEQ ID NO: 16 and SEQ ID NO: 3 is SEQ ID NO: 18. In some embodiments, SEQ ID NO: 1 is SEQ ID NO: 15, SEQ ID NO: 2 is SEQ ID NO: 19 and SEQ ID NO: 3 is SEQ ID NO: 17. In some embodiments, SEQ ID NO: 1 is SEQ ID NO: 15, SEQ ID NO: 2 is SEQ ID NO: 19 and SEQ ID NO: 3 is SEQ ID NO: 18. In some embodiments, SEQ ID NO: 1 is SEQ ID NO: 20, SEQ ID NO: 2 is SEQ ID NO: 16 and SEQ ID NO: 3 is SEQ ID NO: 17. In some embodiments, SEQ ID NO: 1 is SEQ ID NO: 20, SEQ ID NO: 2 is SEQ ID NO: 16 and SEQ ID NO: 3 is SEQ ID NO: 18. In some embodiments, SEQ ID NO: 1 is SEQ ID NO: 20, SEQ ID NO: 2 is SEQ ID NO: 19 and SEQ ID NO: 3 is SEQ ID NO: 17. In some embodiments, the antibody or antigen binding fragment is 5E5. In some embodiments, SEQ ID NO: 1 is SEQ ID NO: 20, SEQ ID NO: 2 is SEQ ID NO: 19 and SEQ ID NO: 3 is SEQ ID NO: 18. It will be understood that antibodies or antigen binding domain that contain SEQ ID NO: 16 by the Chothia system will contain SEQ ID NO: 32 by the Kabat system. Similarly, antibodies or antigen binding domain that contain SEQ ID NO: 19 by the Chothia system will contain SEQ ID NO: 33 by the Kabat system.

In some embodiments, the antibody or antigen binding fragment comprises a heavy chain. In some embodiments, the heavy chain is an IgG heavy chain. In some embodiments, the IgG is IgG4. In some embodiments, the heavy chain comprises an IgG4 constant region. In some embodiments, the IgG4 constant region comprises a sequence with at least 70, 75, 80, 85, 90, 95, 93, 95, 97, 99 or 100% identity to ASTKGPSVFPLAPCSRSTSESTAALGCLVKDYFPEPVTVSWNSGALTSGVHTFPAV LQSSGLYSLSSVVTVPSSSLGTKTYTCNVDHKPSNTKVDKRVESKYGPPCPPCPAP EFEGGPSVFLFSPKPKDTLMISRTPEVTCVVVDVSQEDPEVQFNWYVDGVEVHNA KTKPREEQFNSTYRVVSVLTVLHQDWLNGKEYKCKVSNKGLPSSIEKTISKAKGQ PREPQVYTLPPSQEEMTKNQVSLTCLVKGFYPSDIAVEWESNGQPENNYKTTPPVL DSDGSFFLYSRLTVDKSRWQEGNVFSCSVMHEALHNHYTQKSLSLSLGK (SEQ ID NO: 21). Each possibility represents a separate embodiment of the invention. In some embodiments, the IgG4 constant region comprises a sequence with at least 80% identity to SEQ ID NO: 21. In some embodiments, the IgG4 constant region comprises a sequence with at least 90% identity to SEQ ID NO: 21. In some embodiments, the IgG4 constant region does not induce ADCC or CDC. In some embodiments, the IgG4 constant region comprises a mutation of serine 124 to proline (S124P). In some embodiments, the numbering is with respect to SEQ ID NO: 21. In some embodiments, S124P is S241P, and the numbering is with respect to a full heavy chain. In some embodiments, the IgG4 constant region comprises a mutation of leucine 131 to glutamic acid (L131E). In some embodiments, the numbering is with respect to SEQ ID NO: 21. In some embodiments, L131E is L248E, and the numbering is with respect to a full heavy chain. It will be understood that a depending on deletions or insertions into the full heavy chain the numbering provided for these two mutations may be shifted slightly.

In some embodiments, the antibody or antigen binding fragment comprises a light chain. In some embodiments, the light chain is kappa light chain. In some embodiments, the light chain is lambda light chain. In some embodiments, the antibody or antigen binding fragment comprises a kappa constant region. In some embodiments, the kappa constant region comprises a sequence with at least 70, 75, 80, 85, 90, 95, 93, 95, 97, 99 or 100% identity to RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 22). Each possibility represents a separate embodiment of the invention. In some embodiments, the kappa constant region comprises a sequence with at least 80% identity to SEQ ID NO: 22. In some embodiments, the kappa constant region comprises a sequence with at least 90% identity to SEQ ID NO: 22.

In some embodiments, the antibody or antigen binding fragment comprises a heavy chain variable region comprising or consisting of the amino acid sequence of EVQLQQSGPELVKPGASVKISCKASGYSFTGFYIDWVKQSPGKSLEWIGYIFPSNGE TSYNQKFKGKATLTVDKSSSTVNMQLNSLTSEDSAVYYCARQAFYYFDYWGQGT TLTVSS (SEQ ID NO: 34). In some embodiments, the antibody or antigen binding fragment comprises a light chain variable region comprising or consisting of the amino acid sequence of DIVMSQSPSSLAVSVGEKVTMSCKSSQSLLSSSNQKNYLAWYQQKPGQSPKLLIY WASTRESGVPDRFAGSGSGTDFTLTISSVKAEDLAVYYCQQYYSYPLTFGAGTKL ELK (SEQ ID NO: 35). In some embodiments, the antibody or antigen binding fragment comprises a heavy chain comprising a variable region comprising or consisting of SEQ ID NO: 34 and a light chain comprising a variable region comprising or consisting of SEQ ID NO: 35. In some embodiments, the antibody or antigen binding fragment is a mouse antibody. In some embodiments, the antibody or antigen binding fragment is 5E5.

In some embodiments, the antibody or antigen binding fragment comprises a heavy chain variable region comprising or consisting of the amino acid sequence of EVQLQQSGPELVKPGASVKISCKASGYSFTGFYIDWVKQSPGKSLEWIGYIFPSNGE TSYNQKFKGKATLTVDKSSSTVNMQLNSLTSEDSAVYYCARQAFYYFDYWGQGT TLTVSS (SEQ ID NO: 36). In some embodiments, the antibody or antigen binding fragment comprises a light chain variable region comprising or consisting of the amino acid sequence of DIVMSQSPSSLAVSVGEKVTMSCKSSQSLLSSSNQKNYLAWYQQKPGQSPKLLIY WASTRESGVPDRFAGSGSGTDFTLTISSVKAEDLAVYYCQQYYSYPLTFGAGTKL ELK (SEQ ID NO: 37). In some embodiments, the antibody or antigen binding fragment comprises a heavy chain comprising a variable region comprising or consisting of SEQ ID NO: 36 and a light chain comprising a variable region comprising or consisting of SEQ ID NO: 37. In some embodiments, the antibody or antigen binding fragment is a chimeric antibody. In some embodiments, the antibody or antigen binding fragment is 5E5.

In some embodiments, the antibody or antigen binding fragment comprises a heavy chain variable region comprising or consisting of the amino acid sequence of QVQLVQSGAEVKKPGASVKVSCKASGYSFSGFYIDWVKQPPGKGLEWIGYIFPSS GETSYNQKFKGRVTMTVDKSTSTVYMELSSLRSEDTAVYYCARQAFYYFDYWGQ GTTVTVSS (SEQ ID NO: 38). In some embodiments, the antibody or antigen binding fragment comprises a light chain variable region comprising or consisting of the amino acid sequence of DIVMTQSPDSLAVSLGERATINCKSSQSLLSSSNQKNYLAWYQQKPGQAPRLLIYW ASTRESGVPDRFAGSGSGTDFTLTISSLQAEDVAVYYCQQYYSYPLTFGQGTKLEI K (SEQ ID NO: 39). In some embodiments, the antibody or antigen binding fragment comprises a heavy chain comprising a variable region comprising or consisting of SEQ ID NO: 38 and a light chain comprising a variable region comprising or consisting of SEQ ID NO: 39. In some embodiments, the antibody or antigen binding fragment is a humanized antibody. In some embodiments, the antibody or antigen binding fragment is 5E5.

In some embodiments, the antibody or antigen binding fragment comprises a heavy chain variable region comprising or consisting of the amino acid sequence of EVQLVQSGAEVKKPGASVKVSCKASGYSFTGFYIDWVKQPPGKGLEWIGYIFPSN GETSYNQKFKGRATMTVDKSTSTVYMELSSLRSEDTAVYYCARQAFYYFDYWGQ GTTVTVSS (SEQ ID NO: 40). In some embodiments, the antibody or antigen binding fragment comprises a light chain variable region comprising or consisting of the amino acid sequence of DIVMTQSPDSLAVSLGERATINCKSSQSLLSSSNQKNYLAWYQQKPGQPPKLLIYW ASTRESGVPDRFAGSGSGTDFTLTISSLQAEDVAVYYCQQYYSYPLTFGQGTKLEI K (SEQ ID NO: 41). In some embodiments, the antibody or antigen binding fragment comprises a heavy chain comprising a variable region comprising or consisting of SEQ ID NO: 40 and a light chain comprising a variable region comprising or consisting of SEQ ID NO: 41. In some embodiments, the antibody or antigen binding fragment is a humanized antibody.

In some embodiments, the antibody or antigen binding fragment comprises a heavy chain variable region comprising or consisting of the amino acid sequence of QVQLVQSGAEVKKPGASVKVSCKASGYSFSGFYIDWVKQPPGKGLEWIGYIFPSN GETSYNQKFKGRVTMTVDKSTSTVYMELSSLRSEDTAVYYCARQAFYYFDYWGQ GTTVTVSS (SEQ ID NO: 42). In some embodiments, the antibody or antigen binding fragment comprises a heavy chain comprising a variable region comprising or consisting of SEQ ID NO: 42 and a light chain comprising a variable region comprising or consisting of SEQ ID NO: 41. In some embodiments, the antibody or antigen binding fragment is a humanized antibody.

In some embodiments, the antibody or antigen binding fragment comprises a heavy chain comprising a variable region comprising or consisting of SEQ ID NO: 42 and a light chain comprising a variable region comprising or consisting of SEQ ID NO: 39. In some embodiments, the antibody or antigen binding fragment is a humanized antibody.

In some embodiments, the antibody or antigen binding fragment comprises a heavy chain variable region comprising or consisting of the amino acid sequence of QVQLVQSGAEVKKPGASVKVSCKASGYSFTGFYIDWVKQPPGKGLEWIGYIFPSN GETSYNQKFKGRVTMTVDKSTSTVYMELSSLRSEDTAVYYCARQAFYYFDSWGQ GTTVTVSS (SEQ ID NO: 43). In some embodiments, the antibody or antigen binding fragment comprises a heavy chain comprising a variable region comprising or consisting of SEQ ID NO: 43 and a light chain comprising a variable region comprising or consisting of SEQ ID NO: 39. In some embodiments, the antibody or antigen binding fragment is a humanized antibody.

In some embodiments, the antibody or antigen binding fragment comprises a heavy chain variable region comprising or consisting of the amino acid sequence of EVQLVQSGAEVKKPGASVKVSCKASGYSFSGFYIDWVKQPPGKGLEWIGYIFPSN GETSYNQKFKGRATMTVDKSTSTVYMELSSLRSEDTAVYYCARQAFYYFDYWGQ GTTVTVSS (SEQ ID NO: 44). In some embodiments, the antibody or antigen binding fragment comprises a light chain variable region comprising or consisting of the amino acid sequence of DIVMTQSPDSLAVSLGERATINCKSSQSLLSSSNQKNYLAWYQQKPGQPPKLLIYW ASTRESGVPDRESGSGSGTDFTLTISSLQAEDVAVYYCQQYYSYPLTFGQGTKLEIK (SEQ ID NO: 45). In some embodiments, the antibody or antigen binding fragment comprises a heavy chain comprising a variable region comprising or consisting of SEQ ID NO: 44 and a light chain comprising a variable region comprising or consisting of SEQ ID NO: 39. In some embodiments, the antibody or antigen binding fragment comprises a heavy chain comprising a variable region comprising or consisting of SEQ ID NO: 44 and a light chain comprising a variable region comprising or consisting of SEQ ID NO: 45. In some embodiments, the antibody or antigen binding fragment is a humanized antibody.

In some embodiments, the antibody or antigen binding fragment comprises a heavy chain variable region comprising or consisting of the amino acid sequence of EVQLVQSGAEVKKPGASVKVSCKASGYSFTGFYIDWVKQPPGKGLEWIGYIFPSS GETSYNQKFKGRATMTVDKSTSTVYMELSSLRSEDTAVYYCARQAFYYFDYWGQ GTTVTVSS (SEQ ID NO: 46). In some embodiments, the antibody or antigen binding fragment comprises a heavy chain comprising a variable region comprising or consisting of SEQ ID NO: 46 and a light chain comprising a variable region comprising or consisting of SEQ ID NO: 39. In some embodiments, the antibody or antigen binding fragment is a humanized antibody.

In some embodiments, the antibody or antigen binding fragment comprises a heavy chain variable region comprising or consisting of the amino acid sequence of EVQLVQSGAEVKKPGASVKVSCKASGYSFSGFYIDWVKQPPGKGLEWIGYIFPSSG ETSYNQKFKGRATMTVDKSTSTVYMELSSLRSEDTAVYYCARQAFYYFDYWGQG TTVTVSS (SEQ ID NO: 47). In some embodiments, the antibody or antigen binding fragment comprises a heavy chain comprising a variable region comprising or consisting of SEQ ID NO: 47 and a light chain comprising a variable region comprising or consisting of SEQ ID NO: 39. In some embodiments, the antibody or antigen binding fragment is a humanized antibody.

In some embodiments, the antibody or antigen binding fragment comprises a heavy chain variable region comprising or consisting of the amino acid sequence of QVQLVQSGAEVKKPGASVKVSCKASGYSFSGFYIDWVKQPPGKGLEWIGYIFPSS GETSYNQKFKGRVTMTVDKSTSTVYMELSSLRSEDTAVYYCARQAFYYFDSWGQ GTTVTVSS (SEQ ID NO: 48). In some embodiments, the antibody or antigen binding fragment comprises a heavy chain comprising a variable region comprising or consisting of SEQ ID NO: 48 and a light chain comprising a variable region comprising or consisting of SEQ ID NO: 39. In some embodiments, the antibody or antigen binding fragment is a humanized antibody.

In some embodiments, the antibody or antigen binding fragment comprises a heavy chain constant region comprising or consisting of the amino acid sequence of EVQLQQSGPELVKPGASVKISCKASGYSFTGFYIDWVKQSPGKSLEWIGYIFPSNGE TSYNQKFKGKATLTVDKSSSTVNMQLNSLTSEDSAVYYCARQAFYYFDYWGQGT TLTVSSAKTTPPSVYPLAPGSAAQTNSMVTLGCLVKGYFPEPVTVTWNSGSLSSGV HTFPAVLQSDLYTLSSSVTVPSSTWPSETVTCNVAHPASSTKVDKKIVPRDCGCKP CICTVPEVSSVFIFPPKPKDVLTITLTPKVTCVVVDISKDDPEVQFSWFVDDVEVHT AQTQPREEQFNSTFRSVSELPIMHQDWLNGKEFKCRVNSAAFPAPIEKTISKTKGRP KAPQVYTIPPPKEQMAKDKVSLTCMITDFFPEDITVEWQWNGQPAENYKNTQPIM DTDGSYFIYSKLNVQKSNWEAGNTFTCSVLHEGLHNHHTEKSLSHSPGK (SEQ ID NO: 7). In some embodiments, the antibody or antigen binding fragment comprises a light chain comprising or consisting of the amino acid sequence of DIVMSQSPSSLA VSVGEKVTMSCKSSQSLLSSSNQKNYLAWYQQKPGQSPKLLIY WASTRESGVPDRFAGSGSGTDFTLTISSVKAEDLA VYYCQQYYSYPLTFGAGTKL ELKRADAAPTVSIFPPSSEQLTSGGASVVCFLNNFYPKDINVKWKIDGSERQNGVL NSWTDQDSKDSTYSMSSTLTLTKDEYERHNSYTCEATHKTSTSPIVKSFNRNEC (SEQ ID NO: 8). In some embodiments, the antibody or antigen binding fragment comprises a heavy chain comprising or consisting of SEQ ID NO: 7 and a light chain comprising or consisting of SEQ ID NO: 8. In some embodiments, the antibody or antigen binding fragment is a mouse antibody.

In some embodiments, the antibody or antigen binding fragment comprises a heavy chain comprising or consisting of the amino acid sequence of EVQLQQSGPELVKPGASVKISCKASGYSFTGFYIDWVKQSPGKSLEWIGYIFPSNGE TSYNQKFKGKATLTVDKSSSTVNMQLNSLTSEDSAVYYCARQAFYYFDYWGQGT TLTVSSASTKGPSVFPLAPCSRSTSESTAALGCLVKDYFPEPVTVSWNSGALTSGV HTFPAVLQSSGLYSLSSVVTVPSSSLGTKTYTCNVDHKPSNTKVDKRVESKYGPPC PPCPAPEFLGGPSVFLFPPKPKDTLMISRTPEVTCVVVDVSQEDPEVQFNWYVDGV EVHNAKTKPREEQFNSTYRVVSVLTVLHQDWLNGKEYKCKVSNKGLPSSIEKTIS KAKGQPREPQVYTLPPSQEEMTKNQVSLTCLVKGFYPSDIA VEWESNGQPENNYK TTPPVLDSDGSFFLYSRLTVDKSRWQEGNVFSCSVMHEALHNHYTQKSLSLSLGK (SEQ ID NO: 9). In some embodiments, the antibody or antigen binding fragment comprises a light chain comprising or consisting of the amino acid sequence of DIVMSQSPSSLA VSVGEKVTMSCKSSQSLLSSSNQKNYLAWYQQKPGQSPKLLIY WASTRESGVPDRFAGSGSGTDFTLTISSVKAEDLAVYYCQQYYSYPLTFGAGTKL ELKRTVAAPSVFIFPPSDEQLKSGTASVVCLLNNFYPREAKVQWKVDNALQSGNS QESVTEQDSKDSTYSLSSTLTLSKADYEKHKVYACEVTHQGLSSPVTKSFNRGEC (SEQ ID NO: 10). In some embodiments, the antibody or antigen binding fragment comprises a heavy chain comprising or consisting of SEQ ID NO: 9 and a light chain comprising or consisting of SEQ ID NO: 10. In some embodiments, the antibody or antigen binding fragment is a chimeric antibody.

In some embodiments, the antibody or antigen binding fragment comprises a heavy chain comprising the or consisting of amino acid sequence of QVQLVQSGAEVKKPGASVKVSCKASGYSFSGFYIDWVKQPPGKGLEWIGYIFPSS GETSYNQKFKGRVTMTVDKSTSTVYMELSSLRSEDTAVYYCARQAFYYFDYWGQ GTTVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKYGP PCPPCPAPEFEGGPSVFLFPPKPKDTLMISRTPEVTCVVVDVSQEDPEVQFNWYVD GVEVHNAKTKPREEQFNSTYRVVSVLTVLHQDWLNGKEYKCKVSNKGLPSSIEKT ISKAKGQPREPQVYTLPPSQEEMTKNQVSLTCLVKGFYPSDIA VEWESNGQPENNY KTTPPVLDSDGSFFLYSRLTVDKSRWQEGNVFSCSVMHEALHNHYTQKSLSLSLG K (SEQ ID NO: 11). In some embodiments, the antibody or antigen binding fragment comprises a light chain comprising or consisting of the amino acid sequence of DIVMTQSPDSLAVSLGERATINCKSSQSLLSSSNQKNYLAWYQQKPGQAPRLLIYW ASTRESGVPDRFAGSGSGTDFTLTISSLQAEDVAVYYCQQYYSYPLTFGQGTKLEI KRTVAAPSVFIFPPSDEQLKSGTASVVCLLNNFYPREAKVQWKVDNALQSGNSQE SVTEQDSKDSTYSLSSTLTLSKADYEKHKVYACEVTHQGLSSPVTKSFNRGEC (SEQ ID NO: 12). In some embodiments, the antibody or antigen binding fragment comprises a heavy chain comprising or consisting of SEQ ID NO: 11 and a light chain comprising or consisting of SEQ ID NO: 12. In some embodiments, the antibody or antigen binding fragment is a humanized antibody.

In some embodiments, the antibody or antigen binding fragment comprises a heavy chain or consisting of the amino acid sequence comprising of EVQLVQSGAEVKKPGASVKVSCKASGYSFTGFYIDWVKQPPGKGLEWIGYIFPSN GETSYNQKFKGRATMTVDKSTSTVYMELSSLRSEDTAVYYCARQAFYYFDYWGQ GTTVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKYGP PCPPCPAPEFEGGPSVFLFPPKPKDTLMISRTPEVTCVVVDVSQEDPEVQFNWYVD GVEVHNAKTKPREEQFNSTYRVVSVLTVLHQDWLNGKEYKCKVSNKGLPSSIEKT ISKAKGQPREPQVYTLPPSQEEMTKNQVSLTCLVKGFYPSDIAVEWESNGQPENNY KTTPPVLDSDGSFFLYSRLTVDKSRWQEGNVFSCSVMHEALHNHYTQKSLSLSLG K (SEQ ID NO: 23). In some embodiments, the antibody or antigen binding fragment comprises a light chain comprising or consisting of the amino acid sequence of DIVMTQSPDSLAVSLGERATINCKSSQSLLSSSNQKNYLAWYQQKPGQPPKLLIYW ASTRESGVPDRFAGSGSGTDFTLTISSLQAEDVAVYYCQQYYSYPLTFGQGTKLEI KRTVAAPSVFIFPPSDEQLKSGTASVVCLLNNFYPREAKVQWKVDNALQSGNSQE SVTEQDSKDSTYSLSSTLTLSKADYEKHKVYACEVTHQGLSSPVTKSFNRGEC (SEQ ID NO: 24). In some embodiments, the antibody or antigen binding fragment comprises a heavy chain comprising or consisting of SEQ ID NO: 23 and a light chain comprising or consisting of SEQ ID NO: 24. In some embodiments, the antibody or antigen binding fragment is a humanized antibody.

In some embodiments, the antibody or antigen binding fragment comprises a heavy chain comprising the or consisting of amino acid sequence of QVQLVQSGAEVKKPGASVKVSCKASGYSFSGFYIDWVKQPPGKGLEWIGYIFPSN GETSYNQKFKGRVTMTVDKSTSTVYMELSSLRSEDTAVYYCARQAFYYFDYWGQ GTTVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKYGP PCPPCPAPEFEGGPSVFLFPPKPKDTLMISRTPEVTCVVVDVSQEDPEVQFNWYVD GVEVHNAKTKPREEQFNSTYRVVSVLTVLHQDWLNGKEYKCKVSNKGLPSSIEKT ISKAKGQPREPQVYTLPPSQEEMTKNQVSLTCLVKGFYPSDIAVEWESNGQPENNY KTTPPVLDSDGSFFLYSRLTVDKSRWQEGNVFSCSVMHEALHNHYTQKSLSLSLG K (SEQ ID NO: 25). In some embodiments, the antibody or antigen binding fragment comprises a heavy chain comprising or consisting of SEQ ID NO: 25 and a light chain comprising or consisting of SEQ ID NO: 24. In some embodiments, the antibody or antigen binding fragment is a humanized antibody.

In some embodiments, the antibody or antigen binding fragment comprises a heavy chain comprising a variable region comprising or consisting of SEQ ID NO: 25 and a light chain comprising a variable region comprising or consisting of SEQ ID NO: 12. In some embodiments, the antibody or antigen binding fragment is a humanized antibody.

In some embodiments, the antibody or antigen binding fragment comprises a heavy chain comprising or consisting of the amino acid sequence of QVQLVQSGAEVKKPGASVKVSCKASGYSFTGFYIDWVKQPPGKGLEWIGYIFPSN GETSYNQKFKGRVTMTVDKSTSTVYMELSSLRSEDTAVYYCARQAFYYFDSWGQ GTTVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKYGP PCPPCPAPEFEGGPSVFLFPPKPKDTLMISRTPEVTCVVVDVSQEDPEVQFNWYVD GVEVHNAKTKPREEQFNSTYRVVSVLTVLHQDWLNGKEYKCKVSNKGLPSSIEKT ISKAKGQPREPQVYTLPPSQEEMTKNQVSLTCLVKGFYPSDIAVEWESNGQPENNY KTTPPVLDSDGSFFLYSRLTVDKSRWQEGNVFSCSVMHEALHNHYTQKSLSLSLG K (SEQ ID NO: 26). In some embodiments, the antibody or antigen binding fragment comprises a heavy chain comprising a variable region comprising or consisting of SEQ ID NO: 26 and a light chain comprising a variable region comprising or consisting of SEQ ID NO: 12. In some embodiments, the antibody or antigen binding fragment is a humanized antibody.

In some embodiments, the antibody or antigen binding fragment comprises a heavy chain comprising or consisting of the amino acid sequence of EVQLVQSGAEVKKPGASVKVSCKASGYSFSGFYIDWVKQPPGKGLEWIGYIFPSN GETSYNQKFKGRATMTVDKSTSTVYMELSSLRSEDTAVYYCARQAFYYFDYWGQ GTTVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKYGP PCPPCPAPEFEGGPSVFLFPPKPKDTLMISRTPEVTCVVVDVSQEDPEVQFNWYVD GVEVHNAKTKPREEQFNSTYRVVSVLTVLHQDWLNGKEYKCKVSNKGLPSSIEKT ISKAKGQPREPQVYTLPPSQEEMTKNQVSLTCLVKGFYPSDIAVEWESNGQPENNY KTTPPVLDSDGSFFLYSRLTVDKSRWQEGNVFSCSVMHEALHNHYTQKSLSLSLG K (SEQ ID NO: 27). In some embodiments, the antibody or antigen binding fragment comprises a light chain comprising or consisting of the amino acid sequence of DIVMTQSPDSLAVSLGERATINCKSSQSLLSSSNQKNYLAWYQQKPGQPPKLLIYW ASTRESGVPDRFSGSGSGTDFTLTISSLQAEDVAVYYCQQYYSYPLTFGQGTKLEIK RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 28). In some embodiments, the antibody or antigen binding fragment comprises a heavy chain comprising a variable region comprising or consisting of SEQ ID NO: 27 and a light chain comprising a variable region comprising or consisting of SEQ ID NO: 12. In some embodiments, the antibody or antigen binding fragment comprises a heavy chain comprising a variable region comprising or consisting of SEQ ID NO: 27 and a light chain comprising a variable region comprising or consisting of SEQ ID NO: 28. In some embodiments, the antibody or antigen binding fragment is a humanized antibody.

In some embodiments, the antibody or antigen binding fragment comprises a heavy chain comprising or consisting of the amino acid sequence of EVQLVQSGAEVKKPGASVKVSCKASGYSFTGFYIDWVKQPPGKGLEWIGYIFPSS GETSYNQKFKGRATMTVDKSTSTVYMELSSLRSEDTAVYYCARQAFYYFDYWGQ GTTVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKYGP PCPPCPAPEFEGGPSVFLFPPKPKDTLMISRTPEVTCVVVDVSQEDPEVQFNWYVD GVEVHNAKTKPREEQFNSTYRVVSVLTVLHQDWLNGKEYKCKVSNKGLPSSIEKT ISKAKGQPREPQVYTLPPSQEEMTKNQVSLTCLVKGFYPSDIAVEWESNGQPENNY KTTPPVLDSDGSFFLYSRLTVDKSRWQEGNVFSCSVMHEALHNHYTQKSLSLSLG K (SEQ ID NO: 29). In some embodiments, the antibody or antigen binding fragment comprises a heavy chain comprising a variable region comprising or consisting of SEQ ID NO: 29 and a light chain comprising a variable region comprising or consisting of SEQ ID NO: 12. In some embodiments, the antibody or antigen binding fragment is a humanized antibody.

In some embodiments, the antibody or antigen binding fragment comprises a heavy chain comprising or consisting of the amino acid sequence of EVQLVQSGAEVKKPGASVKVSCKASGYSFSGFYIDWVKQPPGKGLEWIGYIFPSSG ETSYNQKFKGRATMTVDKSTSTVYMELSSLRSEDTAVYYCARQAFYYFDYWGQG TTVTVSSASTKGPSVFPLAPCSRSTSESTAALGCLVKDYFPEPVTVSWNSGALTSG VHTFPAVLQSSGLYSLSSVVTVPSSSLGTKTYTCNVDHKPSNTKVDKRVESKYGPP CPPCPAPEFEGGPSVFLFPPKPKDTLMISRTPEVTCVVVDVSQEDPEVQFNWYVDG VEVHNAKTKPREEQFNSTYRVVSVLTVLHQDWLNGKEYKCKVSNKGLPSSIEKTI SKAKGQPREPQVYTLPPSQEEMTKNQVSLTCLVKGFYPSDIAVEWESNGQPENNY KTTPPVLDSDGSFFLYSRLTVDKSRWQEGNVFSCSVMHEALHNHYTQKSLSLSLG K (SEQ ID NO: 30). In some embodiments, the antibody or antigen binding fragment comprises a heavy chain comprising a variable region comprising or consisting of SEQ ID NO: 30 and a light chain comprising a variable region comprising or consisting of SEQ ID NO: 12. In some embodiments, the antibody or antigen binding fragment is a humanized antibody.

In some embodiments, the antibody or antigen binding fragment comprises a heavy chain comprising or consisting of the amino acid sequence of QVQLVQSGAEVKKPGASVKVSCKASGYSFSGFYIDWVKQPPGKGLEWIGYIFPSS GETSYNQKFKGRVTMTVDKSTSTVYMELSSLRSEDTAVYYCARQAFYYFDSWGQ GTTVTVSSASTKGPSVFPLAPCSRSTSESTAALGCLVKDYFPEPVTVSWNSGALTS GVHTFPAVLQSSGLYSLSSVVTVPSSSLGTKTYTCNVDHKPSNTKVDKRVESKYGP PCPPCPAPEFEGGPSVFLFPPKPKDTLMISRTPEVTCVVVDVSQEDPEVQFNWYVD GVEVHNAKTKPREEQFNSTYRVVSVLTVLHQDWLNGKEYKCKVSNKGLPSSIEKT ISKAKGQPREPQVYTLPPSQEEMTKNQVSLTCLVKGFYPSDIAVEWESNGQPENNY KTTPPVLDSDGSFFLYSRLTVDKSRWQEGNVFSCSVMHEALHNHYTQKSLSLSLG K (SEQ ID NO: 31). In some embodiments, the antibody or antigen binding fragment comprises a heavy chain comprising a variable region comprising or consisting of SEQ ID NO: 31 and a light chain comprising a variable region comprising or consisting of SEQ ID NO: 12. In some embodiments, the antibody or antigen binding fragment is a humanized antibody.

In some embodiments, the antibody or antigen binding fragment thereof comprises three heavy chain CDRs (CDR-H) and three light chain CDRs (CDR-L), wherein: CDR-H1 comprises the amino acid sequence set forth in SEQ ID NO: 49 (SYAMS), CDR-H2 comprises the amino acid sequence as set forth in SEQ ID NO: 50 (AITFGGGNTYYPDSVKG), CDR-H3 comprises the amino acid sequence as set forth in SEQ ID NO: 51 (HGDGNYDFYAMDY), CDR-L1 comprises the amino acid sequence as set forth in SEQ ID NO: 52 (KSSQSLLNSGNQKNYLT), CDR-L2 comprises the amino acid sequence as set forth in SEQ ID NO: 5 (WASTRES), and CDR-L3 comprises the amino acid sequence as set forth in SEQ ID NO: 53 (QNDYSYPLT). In some embodiments, the antibody comprising CDRS: of SEQ ID NO: 49-53 and 5 is 3F6.

In some embodiments, the antibody or antigen binding fragment thereof comprises a heavy chain variable region comprising the amino acid sequence EVKLVESGGGLVKPGGSLKLSCAASGFTFSSYAMSWVRQTPAKRLEWVAAITFGG GNTYYPDSVKGRFTISRDNARNTLYLQMSSLRSEDTAMYYCARHGDGNYDFYAM DYWGQGTSVTVSS (SEQ ID NO: 54). In some embodiments, the antibody or antigen binding fragment thereof comprises a heavy chain variable region consisting of SEQ ID NO: 54. In some embodiments, the antibody or antigen binding fragment thereof comprises a light chain variable region comprising the amino acid sequence DIVMTQSPSSLTVTAGEKVTMSCKSSQSLLNSGNQKNYLTWYQQKPGQPPKLLIY WASTRESGVPDRFTGSGSRTDFTLTISRVQAEDLAVYYCQNDYSYPLTFGAGTKLE LK (SEQ ID NO: 55). In some embodiments, the antibody or antigen binding fragment thereof comprises a light chain variable region consisting of SEQ ID NO: 55. In some embodiments, the antibody or antigen binding fragment thereof comprises a heavy chain variable region comprising or consisting of SEQ ID NO: 54 and a light chain variable region comprising or consisting of SEQ ID NO: 55. In some embodiments, antibody 3F6 comprises a heavy chain variable region comprising or consisting of SEQ ID NO: 54 and a light chain variable region comprising or consisting of SEQ ID NO: 55.

In some embodiments, the antibody or antigen binding fragment thereof comprises three heavy chain CDRs (CDR-H) and three light chain CDRs (CDR-L), wherein: CDR-H1 comprises the amino acid sequence set forth in SEQ ID NO: 49 (SYAMS), CDR-H2 comprises the amino acid sequence as set forth in SEQ ID NO: 56 (TISSDGGNTYYTDSVKG), CDR-H3 comprises the amino acid sequence as set forth in SEQ ID NO: 57 (HDGRGALDY), CDR-L1 comprises the amino acid sequence as set forth in SEQ ID NO: 58 (RASQDISNYLN), CDR-L2 comprises the amino acid sequence as set forth in SEQ ID NO: 59 (YTSRLHS), and CDR-L3 comprises the amino acid sequence as set forth in SEQ ID NO: 60 (QQGNTLPWT). In some embodiments, the antibody comprising CDRS: of SEQ ID NO: 56-60 and 49 is 7A5.

In some embodiments, the antibody or antigen binding fragment thereof comprises a heavy chain variable region comprising the amino acid sequence EVKLVESGGGLVKPGGSLKLSCAASGFTFSSYAMSWVRQTPAKRLEWVATISSDG GNTYYTDSVKGRFTISRDNARNTLDLQMSSLRSEDTAMYYCARHDGRGALDYW GQGTSVTVSS (SEQ ID NO: 61). In some embodiments, the antibody or antigen binding fragment thereof comprises a heavy chain variable region consisting of SEQ ID NO: 61. In some embodiments, the antibody or antigen binding fragment thereof comprises a light chain variable region comprising the amino acid sequence DIQMTQTTSSLSASLGDRVTISCRASQDISNYLNWYQQKPDGTVKLLIYYTSRLHS GVPSRFSGSGSGTDYSLTISNLEQEDIATYFCQQGNTLPWTFGGGTKLEIK (SEQ ID NO: 62). In some embodiments, the antibody or antigen binding fragment thereof comprises a light chain variable region consisting of SEQ ID NO: 62. In some embodiments, the antibody or antigen binding fragment thereof comprises a heavy chain variable region comprising or consisting of SEQ ID NO: 61 and a light chain variable region comprising or consisting of SEQ ID NO: 62. In some embodiments, antibody 7A5 comprises a heavy chain variable region comprising or consisting of SEQ ID NO: 61 and a light chain variable region comprising or consisting of SEQ ID NO: 62.

In some embodiments, the antibody or antigen binding fragment thereof comprises three heavy chain CDRs (CDR-H) and three light chain CDRs (CDR-L), wherein: CDR-H1 comprises the amino acid sequence set forth in SEQ ID NO: 63 (NSAVH), CDR-H2 comprises the amino acid sequence as set forth in SEQ ID NO: 64 (VIWAGGNTNYNSTLMS), CDR-H3 comprises the amino acid sequence as set forth in SEQ ID NO: 65 (HETYGDSFDY), CDR-L1 comprises the amino acid sequence as set forth in SEQ ID NO: 66 (RSSQSLLDSDGKTYLN), CDR-L2 comprises the amino acid sequence as set forth in SEQ ID NO: 67 (LVSKLDS), and CDR-L3 comprises the amino acid sequence as set forth in SEQ ID NO: 68 (WQGTHFPFT). In some embodiments, the antibody comprising CDRS: of SEQ ID NO: 63-68 is 10G12.

In some embodiments, the antibody or antigen binding fragment thereof comprises a heavy chain variable region comprising the amino acid sequence QVQLKESGPVLVAPSQSLSITCTVSGFSLTNSAVHWVRQPPGKGLEWLGVIWAGG NTNYNSTLMSRLTINKDNSKSQVFLRMNSLQTDDTAIYYCAKHETYGDSFDYWG QGTTLTVSS (SEQ ID NO: 69). In some embodiments, the antibody or antigen binding fragment thereof comprises a heavy chain variable region consisting of SEQ ID NO: 69. In some embodiments, the antibody or antigen binding fragment thereof comprises a light chain variable region comprising the amino acid sequence DVVMTQTPLTLSVTIGQPASISCRSSQSLLDSDGKTYLNWLFQRPGQSPKRLIYLVS KLDSGVPDRFTGSGSGTDFTLKISRVEAEDLGVYYCWQGTHFPFTFGSGTKLEIK (SEQ ID NO: 70). In some embodiments, the antibody or antigen binding fragment thereof comprises a light chain variable region consisting of SEQ ID NO: 70. In some embodiments, the antibody or antigen binding fragment thereof comprises a heavy chain variable region comprising or consisting of SEQ ID NO: 69 and a light chain variable region comprising or consisting of SEQ ID NO: 70. In some embodiments, antibody 10G12 comprises a heavy chain variable region comprising or consisting of SEQ ID NO: 69 and a light chain variable region comprising or consisting of SEQ ID NO: 70.

In some embodiments, the antibody or antigen binding fragment thereof is capable of binding ILT3. In some embodiments, the antibody or antigen binding fragment thereof is an anti-ILT3 antibody. In some embodiments, the target antigen of the antibody is ILT3. In some embodiments, ILT3 is membrane ILT3 (mILT3). In some embodiments, ILT3 is soluble ILT3 (sILT3). An “anti-ILT3 antibody”, “an antibody which recognizes ILT3”, or “an antibody against ILT3” is an antibody that binds ILT3, with sufficient affinity and specificity. In some embodiments, the antibody has increased binding to ILT3. In some embodiments, the antibody has increased binding to ILT3 as compared to a commercially available ILT3 antibody. In some embodiments, the commercially available ILT3 antibody is selected from ZM4.1, m52B8 and IO202. In some embodiments, the antibody or antigen-binding fragment thereof does not significantly bind to any other target. In some embodiments, any other target is any other ILT protein. In some embodiments, ILT3 is mammalian ILT3. In some embodiments, ILT3 is human ILT3. In some embodiments, mammalian is monkey. In some embodiments, monkey is selected from rhesus and cynomolgus. In some embodiments, monkey is cynomolgus. In some embodiments, mammalian is human. In some embodiments, mammalian is not murine. In some embodiments, the antibody does not bind murine ILT3. In some embodiments, the antibody binds human and monkey ILT3.

In some embodiments, membranal ILT3 is on the cell surface. In some embodiments, the membrane is the plasma membrane. In some embodiment, the membrane is the membrane of an immune cell. In some embodiments, the immune cell is selected from myeloid cells, dendritic cells, and macrophages. In some embodiments, the immune cell is a myeloid cell. In some embodiments, the immune cell is a dendritic cell. In some embodiments, the immune cell is a macrophage. In some embodiments, the myeloid cell is a myeloid derived suppressor cell (MDSC). In some embodiments, the dendritic cell is a tolerogenic dendritic cell. In some embodiments, the macrophage is a suppressive macrophage. In some embodiments, the macrophage is an M2 macrophage. In some embodiments, an M2 macrophage is a suppressive macrophage. In some embodiments, the immune cell is a tumor associated immune cell. In some embodiments, the immune cell is a tumor infiltrating immune cell. In some embodiments, the macrophage is a tumor associated macrophage (TAM).

As used herein, the terms “increased binding affinity” and “greater binding affinity” are interchangeable. In some embodiments, antibody or antigen-binding portion thereof of the present invention has a greater binding affinity to sCD28 compared to the mCD28. In one embodiment, greater affinity as used herein is by 10%. In one embodiment, greater affinity as used herein is by 30%. In one embodiment, greater affinity as used herein is by 50%. In one embodiment, greater affinity as used herein is by 75%. In one embodiment, greater affinity as used herein is by 100%. In one embodiment, greater affinity as used herein is by 150%. In one embodiment, greater affinity as used herein is by 250%. In one embodiment, greater affinity as used herein is by 500%. In one embodiment, greater affinity as used herein is by 1,000%. In one embodiment, greater affinity as used herein is by 1.5-fold. In one embodiment, greater affinity as used herein is by 2-fold. In one embodiment, greater affinity as used herein is by 5-fold. In one embodiment, greater affinity as used herein is by 10-fold. In one embodiment, greater affinity as used herein is by 50-fold. In one embodiment, greater affinity as used herein is by 100-fold. In one embodiment, greater affinity as used herein is by 500-fold. In one embodiment, greater affinity as used herein is by 1,000-fold.

An “anti-ILT3 antibody”, “an antibody which recognizes ILT3”, or “an antibody against ILT3” is an antibody that binds to the ILT3, with sufficient affinity and specificity. In some embodiments, an anti-ILT3 antibody has ILT3 as the antigen to which it binds.

An “antigen” is a molecule or a portion of a molecule capable of eliciting antibody formation and being bound by an antibody. An antigen may have one or more than one epitope. The specific reaction referred to above is meant to indicate that the antigen will react, in a highly selective manner, with its corresponding antibody and not with the multitude of other antibodies which may be evoked by other antigens.

The term “antigenic determinant” or “epitope” according to the invention refers to the region of an antigen molecule that specifically reacts with particular antibody. Peptide sequences derived from an epitope can be used, alone or in conjunction with a carrier moiety, applying methods known in the art, to immunize animals and to produce additional polyclonal or monoclonal antibodies. Immunoglobulin variable domains can also be analyzed using the IMGT information system (www://imgt.cines.fr/) (IMGT®/V-Quest) to identify variable region segments, including CDRs. Sec, e.g., Brochet, X. et al, Nucl. Acids Res. J6: W503-508 (2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al, U.S. Dept. of Health and Human Services, “Sequence of Proteins of Immunological Interest” (1983).

In some embodiments, the agent is an antibody or antigen binding fragment thereof. In some embodiments, the antigen binding fragment is a Fab fragment. In some embodiments, the antibody is a single domain antibody. In some embodiments, the antibody lacks a Fc domain. In some embodiments, the agent is an antigen binding domain that lacks an Fc domain. In some embodiments, the agent is a single-domain antibody. In some embodiments, the agent is a camelid, shark or nanobody. In some embodiments, the antibody or fragment is fused to another protein or fragment of a protein. In some embodiments, the second protein or fragment increases half-life, particularly in serum. In some embodiments, the half-life extending protein is human serum albumin. In some embodiments, the agent is modified by a chemical that produces a modification that enhances half-life. In some embodiments, the modification is PEGylation and the chemical is polyethylene glycol. A skilled artisan will appreciate that any half-life extending protein or chemical agent, or modification known in the art may be used.

In some embodiments, the binding of the antibody or antigen binding domain to a cell does not kill the cell. In some embodiments, the binding of the antibody or antigen binding domain to a cell does not lead to death of the cell. In some embodiments the antibody or antigen binding domain does not induce antibody dependent cell-mediated cytotoxicity (ADCC). In some embodiments, the antibody or antigen binding domain does not induce complement-dependent cytotoxicity (CDC). In some embodiments, the antibody or antigen binding domain does not induce ADCC and/or CDC. In some embodiments, the antibody or antigen binding domain comprises an IgG2 or IgG4 domain. In some embodiments, the antibody or antigen binding domain comprises an IgG2 domain. In some embodiments, the antibody or antigen binding domain comprises an IgG4 domain. In some embodiments, the antibody or antigen binding domain comprises an IgG1 or IgG3 mutated to reduce cell death mediated by binding of the antibody. In some embodiments, the mutation mutates a Fc receptor binding domain. In some embodiments, a Fc domain of the antibody is engineered or mutated to decrease CDC, ADCC or both. Fc engineering is well known in the art, and any mutation or amino acid change that is known to decrease antibody mediated cell killing may be used.

In some embodiments, the antibody or antigen binding domain does not comprise IgG1 and/or IgG3. In some embodiments, the antibody or antigen binding domain does not induce antibody-dependent cell-mediated cytotoxicity (ADCC). In some embodiments the antibody or antigen binding domain does not induce complement-dependent cytotoxicity (CDC). In some embodiments, the antibody or antigen binding domain comprises an IgG1 or IgG3 comprising a mutation that reduces ADCC, CDC or both induced by the antibody's binding. In some embodiments, the mutation reduces the ADCC, CDC or both to nothing. ADCC and CDC are well characterized and antibody sequences that allow for these cytotoxic pathways to be induced are well known. Mutations, such as for non-limiting examples, mutation of IgG1 or IgG3 to IgG2 or IgG4 are well known. Any such mutation may be used in the backbone of the antibodies of the invention.

In some embodiments, binding ILT3 inhibits binding of ILT3 to a ligand. In some embodiments, binding ILT3 inhibits binding of a ligand to ILT3. In some embodiments, the ligand is an ILT3 ligand. In some embodiments, the ligand is Apolipoprotein E (APOE). In some embodiments, the ligand is fibronectin (FN1). In some embodiments, the ligand is APOE and FN1. In some embodiments, the ligand inhibits binding of ILT3 to both APOE and FN1. In some embodiments, the APOE is present in the tumor microenvironment (TME). In some embodiments, the FN1 is present in the TME. In some embodiments, the APOE is present in extracellular matrix. In some embodiments, the FN1 is present in extracellular matrix. In some embodiments, the extracellular matrix is tumor extracellular matrix.

In some embodiments, the antibody or antigen binding fragment thereof is an immune checkpoint inhibitor (ICI). In some embodiments, the antibody or antigen binding fragment is an ILT3 blocking antibody. In some embodiments, the antibody or antigen binding fragment's binding produces ILT3 blockade. In some embodiments, binding to ILT3 releases immune cells from ILT3-mediated inhibition. In some embodiments, inhibition is immunosuppression. In some embodiments, the immune cell is a dendritic cell. In some embodiments, the immune cell is T cells. In some embodiments, the T cell is a CD8 T cell. In some embodiments, the T cell is a CD4 T cell. In some embodiments, binding of ILT3 releases T cells from ILT3-mediated inhibition. In some embodiments, binding of ILT3 releases monocytes from ILT3-mediated inhibition. In some embodiments, binding of ILT3 releases macrophages from ILT3-mediated inhibition. In some embodiments, binding of ILT3 releases dendritic cells from ILT3-mediated inhibition. In some embodiments, the immune cell is a macrophage. In some embodiments, the macrophages are M0 macrophages. In some embodiments, a M0 macrophage is a non-activated macrophage. In some embodiments, a M0 macrophage is a naïve macrophage. In some embodiments, the macrophages are M1 macrophages. In some embodiments, a M1 macrophage is an inflammatory macrophage. In some embodiments, a M1 macrophage is a pro-inflammatory macrophage. In some embodiments, the macrophages are M2 macrophages. In some embodiments, a M2 macrophage is an anti-inflammatory macrophage. In some embodiments, a M2 macrophage is a suppressive macrophage. In some embodiments, a M2 macrophage is a tolerogenic macrophage. In some embodiments, the monocytes are macrophages. In some embodiments, the macrophages are immature macrophages. In some embodiments, the macrophages are inflammatory macrophages. In some embodiments, the macrophages are tolerogenic macrophages.

In some embodiments, release is decrease. In some embodiments, decrease is a decrease of at least 10, 15, 20, 25, 30, 35, 40, 45, 50, 55, 60, 65, 70, 75, 80, 85, 90, 95, 97, 99 or 100%. Each possibility represents a separate embodiment of the invention. In some embodiments, release comprises immune cell activation. In some embodiments, activation is increased activation. In some embodiments, increased is an increase of at least 10, 15, 20, 25, 30, 35, 40, 45, 50, 55, 60, 65, 70, 75, 80, 85, 90, 95, 97, 100, 125, 150, 175, 200, 250, 300, 350, 400, 450 or 500%. Each possibility represents a separate embodiment of the invention. In some embodiments, dendritic cell activation is increased. In some embodiments, T cell activation is increased. In some embodiments, monocyte activation in increased. In some embodiments, macrophage activation is increased. In some embodiments, increasing T cell activation comprises restoring T cell activation suppressed by MDSCs. In some embodiments, increasing T cell activation comprises restoring T cell activation suppressed by tolerogenic DCs (DCtols). In some embodiments, activation is Fc receptor mediated activation. In some embodiments, increasing DC activation comprises restoring DC activation suppressed by fibronectin or APOE. In some embodiments, increasing monocyte activation comprises restoring monocyte activation suppressed by fibronectin or APOE.

In some embodiments, release comprises increased proliferation. In some embodiments, activation comprises increased proliferation. In some embodiments, release comprises increased pro-inflammatory cytokine secretion. In some embodiments, release comprises increased pro-inflammatory chemokine secretion. In some embodiments, activation comprises increased pro-inflammatory cytokine secretion. In some embodiments, activation comprises increased pro-inflammatory chemokine secretion. In some embodiments, the pro-inflammatory cytokine is selected from TNFa, IFNg and IL-8. In some embodiments, the pro-inflammatory cytokine is TNFa. In some embodiments, the pro-inflammatory cytokine is IFNg. In some embodiments, the pro-inflammatory cytokine is IL-8. In some embodiments, the pro-inflammatory chemokine is selected from CCL3 and CCL4. In some embodiments, the pro-inflammatory chemokine is CCL3. In some embodiments, the pro-inflammatory chemokine is CCL4.

By another aspect, there is provided an antibody or antigen binding fragment thereof that competes with an antibody or antigen binding fragment of the invention for binding to ILT3.

In some embodiments, competing for binding comprises binding the same epitope. Methods of determining the epitope to which an antibody binds are well known in the art and any such method can be used for determining the epitope to which the antibody of the invention binds. In some embodiments, the epitope is within a ligand binding domain. In some embodiments, the epitope is sufficiently close to a ligand binding domain so that binding of the antibody or antigen binding domain occludes, blocks or alters the ligand binding domain. In some embodiments, the epitope comprises low homology to the corresponding mouse sequence of ILT3.

By another aspect, there is provide a nucleic acid molecule that encodes an antibody or antigen binding fragment of the invention.

In some embodiments, a nucleic acid molecule is a plurality of nucleic acid molecules. In some embodiments, the nucleic acid molecule comprises an open reading frame that encodes the antibody or antigen binding fragment of the invention. In some embodiments, an open reading frame is a plurality of open reading frame. In some embodiments, a nucleic acid molecule encodes the heavy chain. In some embodiments, a nucleic acid molecule encodes the light chain. In some embodiments, the same nucleic acid molecule encodes the heavy chain and the light chain. In some embodiments, different nucleic acid molecules encode the heavy chain and the light chain.

In some embodiments, the nucleic acid molecule is plasmid. In some embodiments, the vector is an expression vector. In some embodiments, the vector is configured to expresses in a target cell. In some embodiments, the nucleic acid molecule comprises an open reading frame.

For example, the polynucleotide may encode an entire immunoglobulin molecule chain, such as a light chain or a heavy chain. A complete heavy chain includes not only a heavy chain variable region (VH) but also a heavy chain constant region (CH), which typically will comprise three constant domains: CH1, CH2 and CH3; and a “hinge” region. In some situations, the presence of a constant region is desirable.

Other polypeptides which may be encoded by the polynucleotide include antigen-binding antibody fragments such as single domain antibodies (“dAbs”), Fv, scFv, Fab′ and CHI and CK or CL domain has been excised. As minibodies are smaller than conventional antibodies they should achieve better tissue penetration in clinical/diagnostic use but being bivalent they should retain higher binding affinity than monovalent antibody fragments, such as dAbs. Accordingly, unless the context dictates otherwise, the term “antibody” as used herein encompasses not only whole antibody molecules, but also antigen-binding antibody fragments of the type discussed above. Each framework region present in the encoded polypeptide may comprise at least one amino acid substitution relative to the corresponding human acceptor framework. Thus, for example, the framework regions may comprise, in total, three, four, five, six, seven, eight, nine, ten, eleven, twelve, thirteen, fourteen, or fifteen amino acid substitutions relative to the acceptor framework regions. Given the properties of the individual amino acids comprising the disclosed protein products, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

Suitably, the polynucleotides described herein may be isolated and/or purified. In some embodiments, the polynucleotides are isolated polynucleotides.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring”.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C).

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, lncRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides.

As used herein, the term “oligonucleotide” refers to a short (e.g., no more than 100 bases), chemically synthesized single-stranded DNA or RNA molecule. In some embodiments, oligonucleotides are attached to the 5′ or 3′ end of a nucleic acid molecule, such as by means of ligation reaction.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Expressing of a gene within a cell is well known to one skilled in the art and herein its delivery may be performed by a method of the invention or using a composition of the invention. In some embodiments, the gene is in an expression vector such as plasmid or viral vector. The vector may be a viral vector. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene or open reading frame is operably linked to a promoter or other regulatory element. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell by a method of the invention). In some embodiments, the regulatory element or promoter is active in a target cell. In some embodiments, the target cell is a packaging cell line.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (+), pGL3, pZcoSV2 (+), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-IMTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

By another aspect, there is provided a composition comprising an antibody or antigen binding fragment of the invention.

In some embodiments, the composition is a pharmaceutical composition. In some embodiments, the composition is a therapeutic composition. In some embodiments, the composition is a preventative composition. In some embodiments, the composition comprises a therapeutically effective amount of the antibody or antigen binding domain. In some embodiments, the composition comprises a therapeutically acceptable carrier, excipient or adjuvant.

The term “therapeutically effective amount” refers to an amount of a drug effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition is for use in a method of the invention. In some embodiments, the antibody or antigen binding domain is for use in a method of the invention. In some embodiments, the composition is for use in enhancing immune function. In some embodiments, the antibody or antigen binding domain is for use in enhancing immune function. In some embodiments, enhancing immune function is alleviating immune suppression. In some embodiments, the composition is for use in treating cancer. In some embodiments, the antibody or antigen binding domain is for use in treating cancer.

In some embodiments, the composition is formulated for administration to a subject. In some embodiments, the composition is formulated for systemic administration. In some embodiments, the composition is formulated for intratumoral administration. As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, or intraperitoneal.

The dosage administered will be dependent upon the age, health, and weight of the recipient, kind of concurrent treatment, if any, frequency of treatment, and the nature of the effect desired.

According to another aspect, there is provided a method of increasing immune function in a subject in need thereof, the method comprising administering to the subject a pharmaceutical composition of the invention or an antibody or antigen binding fragment of the invention to the subject, thereby increasing immune function in the subject.

In some embodiments, increasing immune function comprises increasing immune surveillance. In some embodiments, increasing immune function comprises increasing an immune response. In some embodiments, increasing immune function comprises decreasing immunosuppression. In some embodiments, immune function comprises immune mediated cell killing. In some embodiments, the killing is killing of a disease cell. In some embodiments, the immune response is against a disease cell. In some embodiments, the cell is a target cell.

In some embodiments, the disease is a disease treatable by killing a target cell. In some embodiments, the disease is a disease treatable by inducing an immune response against a target cell. In some embodiments, the target cell is a diseased cell. In some embodiments, the disease is characterized by the presence of immune cells expressing ILT3.

In some embodiments, the disease is cancer. In some embodiments, target cell is a cancer cell. In some embodiments, the cancer is a solid cancer. In some embodiments, the cancer is a tumor. In some embodiments, the target cell is a tumor cell. In some embodiments, the cancer is characterized by the presence of immune cells expressing ILT3. In some embodiments, the cancer is characterized by the presence of tumor infiltrating immune cell expressing ILTs. In some embodiments, the cancer is characterized by the expression of APOE, FN1 or both. In some embodiments, the cancer is the cancer extracellular matrix. In some embodiments, the cancer is the cancer TME. In some embodiments, expression is overexpression. In some embodiments, overexpression is as compared to non-disease tissue. In some embodiments, non-disease tissue is non-cancerous tissue. In some embodiments, the tissue is of the same type as the disease tissue. In some embodiments, the tissue is of the same type as the tumor. In some embodiments, of the same tissue type is derived from the same tissue type.

As used herein “cancer” or “pre-malignancy” are diseases associated with cell proliferation. Non-limiting types of cancer include carcinoma, sarcoma, lymphoma, leukemia, blastoma and germ cells tumors.

In some embodiments, the cancer is solid cancer. In some embodiments, the cancer is a tumor. In some embodiments, the cancer is selected from hepato-biliary cancer, cervical cancer, urogenital cancer (e.g., urothelial cancer), testicular cancer, prostate cancer, thyroid cancer, ovarian cancer, nervous system cancer, ocular cancer, lung cancer, soft tissue cancer, bone cancer, pancreatic cancer, bladder cancer, skin cancer, intestinal cancer, hepatic cancer, rectal cancer, colorectal cancer, esophageal cancer, gastric cancer, gastroesophageal cancer, breast cancer (e.g., triple negative breast cancer), renal cancer (e.g., renal carcinoma), skin cancer, head and neck cancer, leukemia and lymphoma. In some embodiments, the cancer is selected from breast cancer, kidney cancer, head and neck cancer, lung cancer, sarcoma, gastric cancer and ovarian cancer. In some embodiments, the cancer is gastric cancer. In some embodiments, the cancer is colon cancer.

In some embodiments, the method further comprises administering an immune checkpoint inhibitor (ICI). In some embodiments, inhibition is blockade. In some embodiments, the method further comprises administering an immunostimulatory agent. In some embodiments, the agent is a molecule. In some embodiments, the agent is an antibody. In some embodiments, the agent is an ICI. In some embodiments, the immunostimulatory agent has specificity for a target selected from CTLA-4, PD-1, PD-LI, PD-L2, CD40, 0X40, CD137, GFTR, ILT2, or LAG3, or ligands of these proteins. In some embodiments, the ICI targets the PD-1/PD-L1 axis. In some embodiments, the ICI inhibits PD-1, PD-L1, PD-L2 or a combination thereof. In some embodiments, the ICI inhibits the PD-1/PD-L1/PD-L2 checkpoint. In some embodiments, the ICI targets PD-1. In some embodiments, the ICI binds PD-1. In some embodiments, the ICI inhibits PD-1. In some embodiments, the anti-PD-1 agent is pembrolizumab. In some embodiments, the anti-PD-1 agent is nivolumab. In some embodiments, the anti-PD-L1 agent is atezolizumab. Other examples of anti-PD-1 antibodies include but are not limited to cemiplimab, dostarlimab, durvalumab and avelumab.

In some embodiments, the antibody or antigen binding fragment of the invention is for use in combination with an immunostimulatory agent. In some embodiments, the composition of the invention is for use in combination with an immunostimulatory agent. In some embodiments, the use is a method of the invention. In some embodiments, the antibody or antigen binding fragment of the invention is for use in production of a medicament for the treatment of a disease. In some embodiments, the composition of the invention is for use in production of a medicament for the treatment of a disease.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Example 1: Breadth of ILT3 Expression

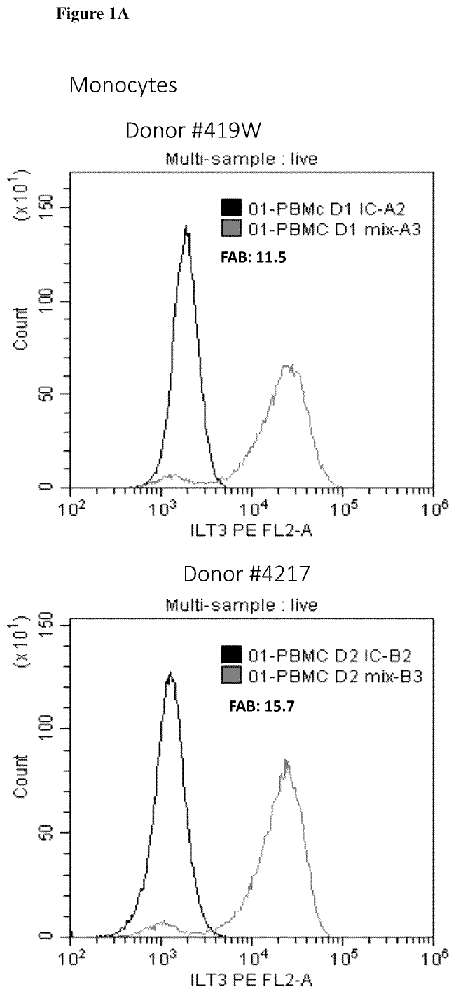

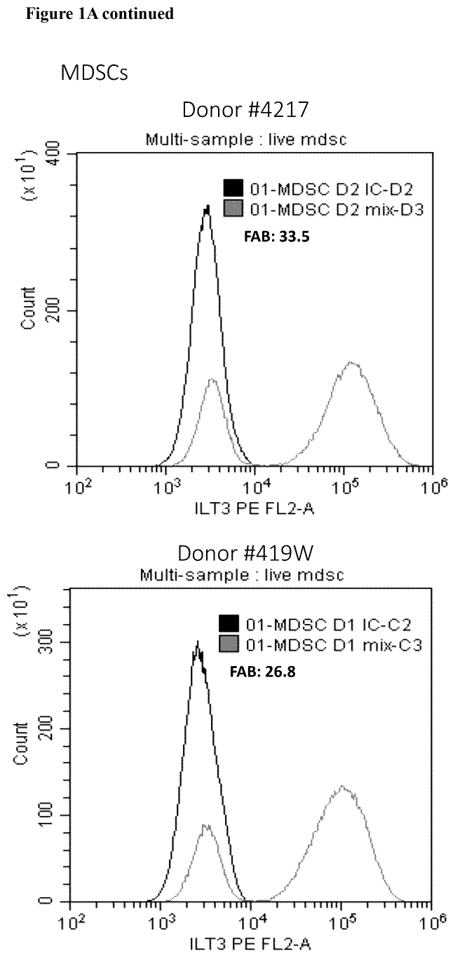

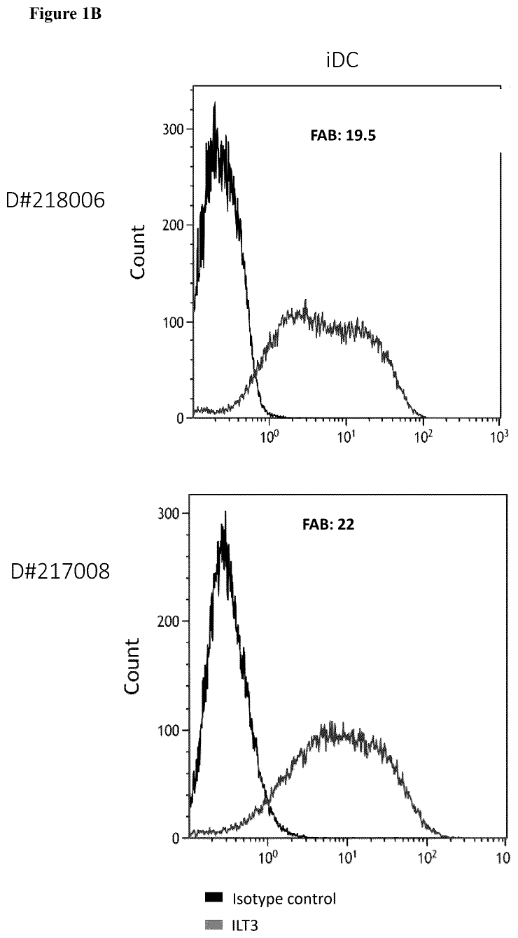

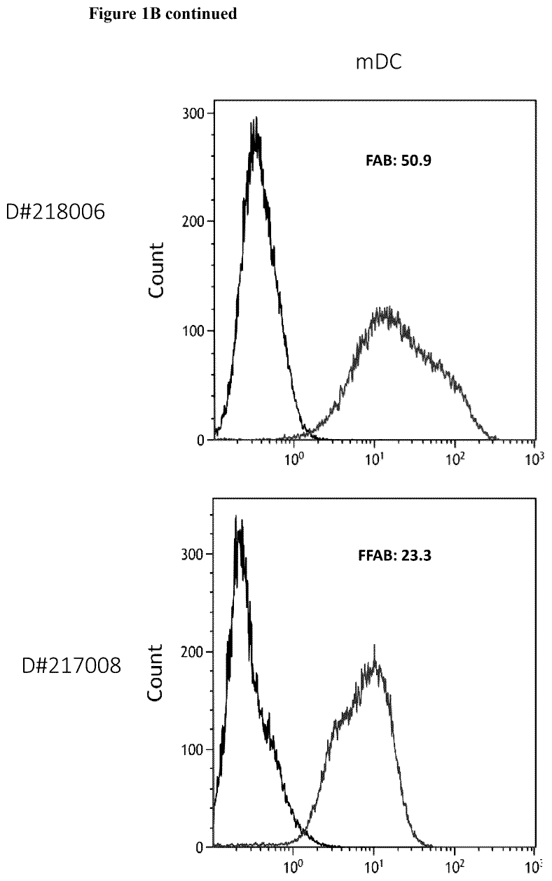

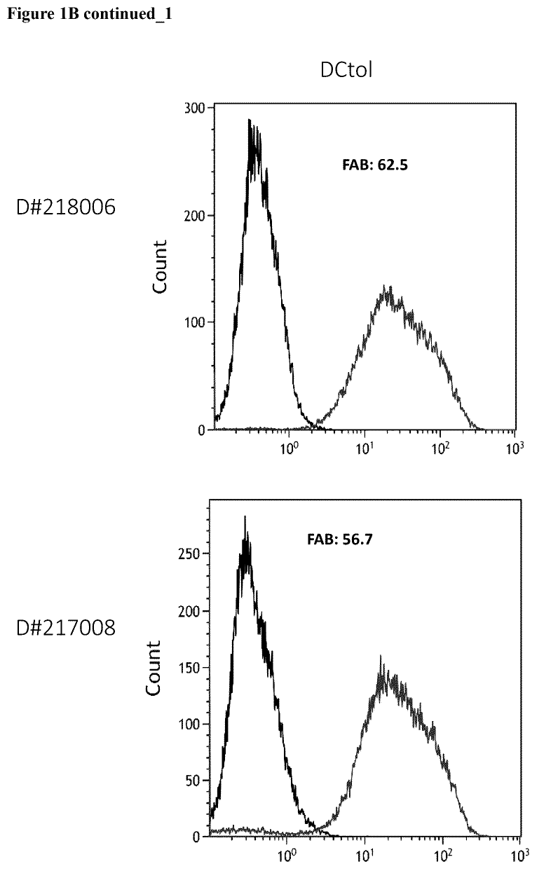

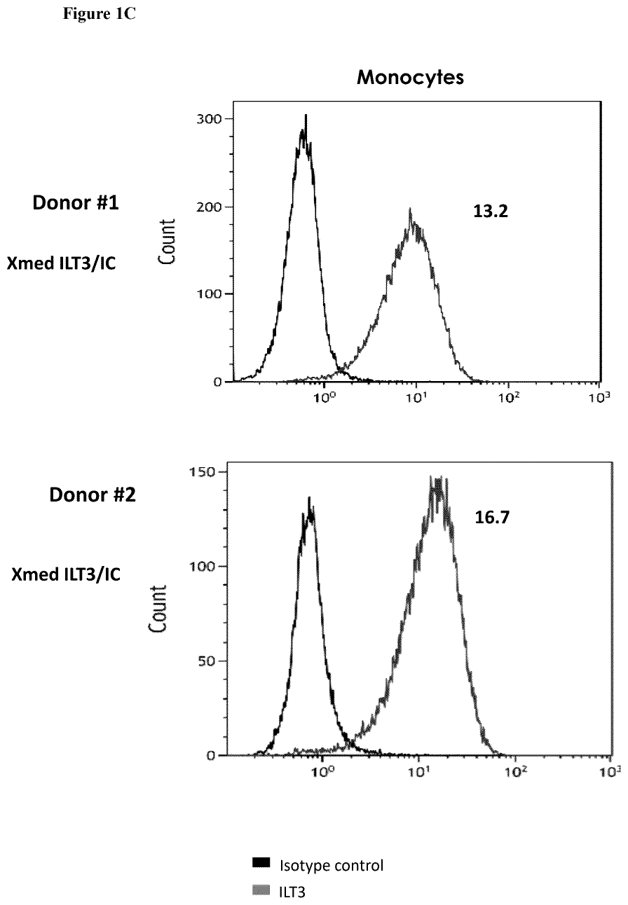

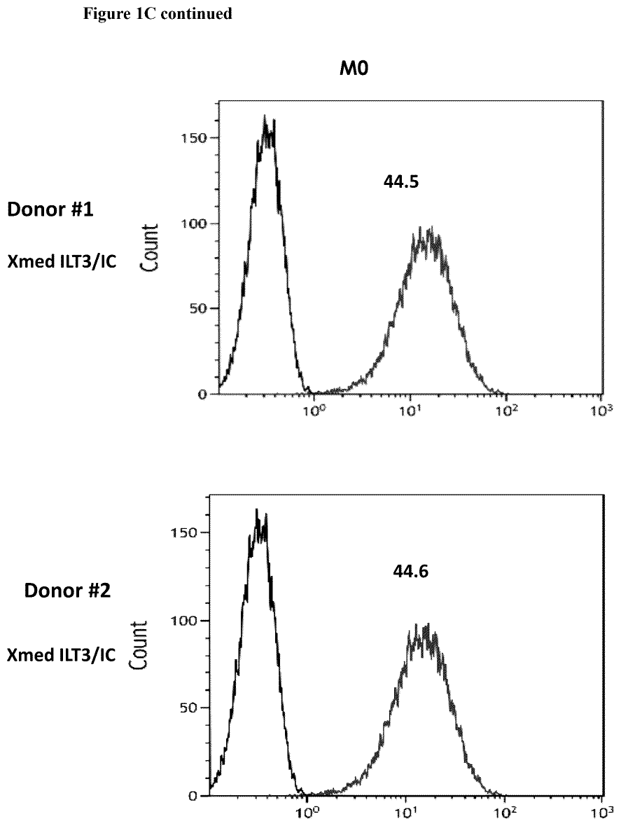

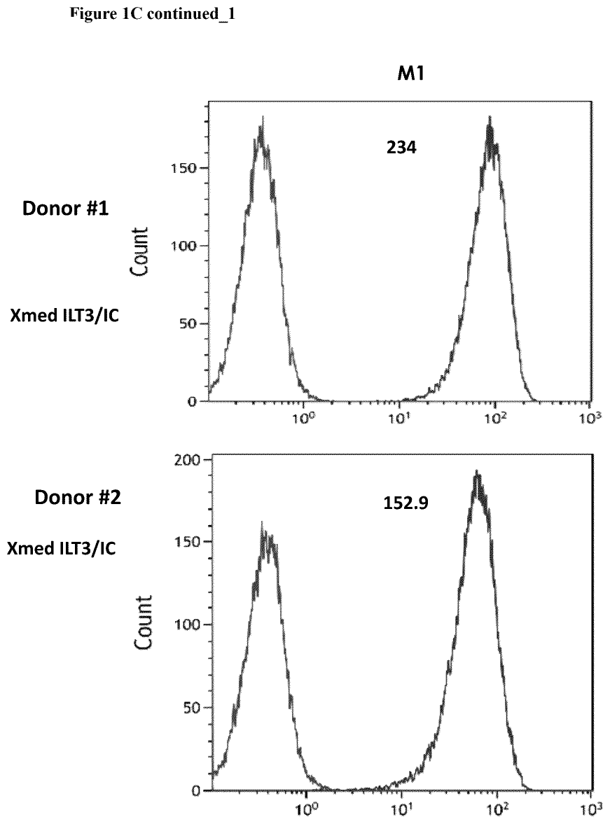

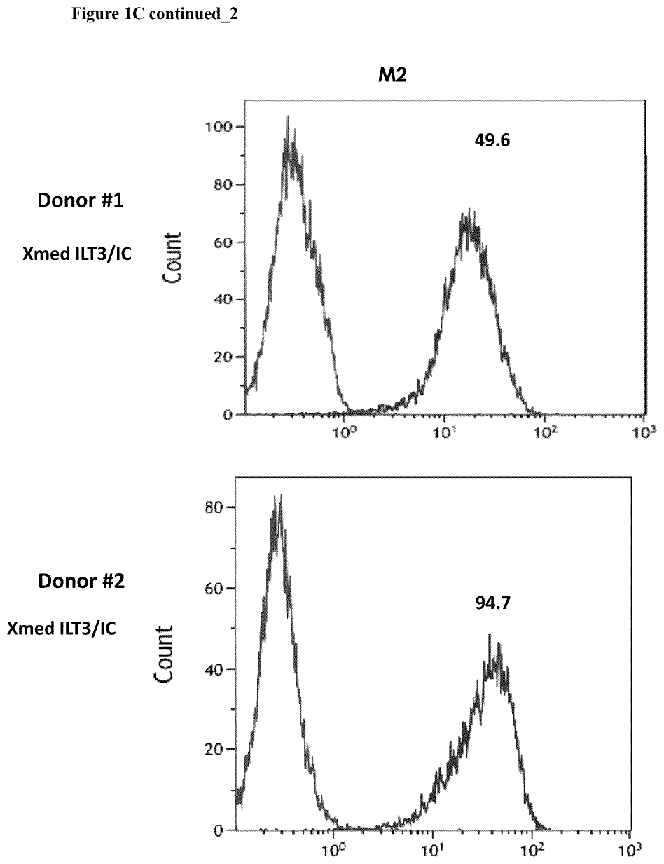

As a starting point, the breadth of ILT3 expression in various immune cells, cancer associated immune cells and cancers was examined. First, PBMCs from two healthy donors were stained with a commercially available anti-ILT3 antibody conjugated to PE (ZM4.1-PE, Biolegend Cat. #333008) and then analyzed by FACS. Monocytes, which were isolated based on CD14 expression, were lowly positive for ILT3 (FIG. 1A). Myeloid derived suppressor cells (MDSCs) were generated from the PBMCs by co-incubation with SK-MEL-5 melanoma cells in the presence of GM-CSF for 7 days. MDSCs were found to have upregulated surface expression of ILT3 as compared to the monocytes (FIG. 1A).

Immature dendritic cells (iDC) were generated from monocytes isolated from two other healthy donors by culturing in the presence of GM-CSF (50 ng/ml) and IL-4 (20 ng/ml) for 6 days. Mature dendritic cells (mDC) were produced by a further culture in the presence of LPS (100 ng/ml) for 48 hours, and tolerogenic DCs (DCtol) were produced by culturing iDC with with IL-10 (100U/ml) and IFNa-2b (1000U/ml) for 48 hours. For both donor's cells the DCtol showed the highest level of surface ILT3 (FIG. 1B).

Monocytes were again isolated from heathy donors and differentiated into various types of macrophages. First, isolated monocytes were cultured with M-CSF (50 ng/ml) for 6 days to produce M0 macrophages. The M0 macrophages were then cultured with IFNg (20 ng/ml) and LPS (100 ng/ml) for 48 hrs to produce M1 macrophages. M2 macrophages were produced by culturing the M0 macrophages with M-CSF (10 ng/ml) and IL4 (20 ng/ml) for 48 hrs. After differentiation expression of CD80, CD206, CD14 and HLA-DR were used to verify the obtained populations. ILT3 levels in these cells were evaluated by FACS. All macrophages (M0, M1 and M2) showed increased ILT3 expression as compared to control monocytes, with M1 macrophages showing a more than 10-fold increase (FIG. 1C).

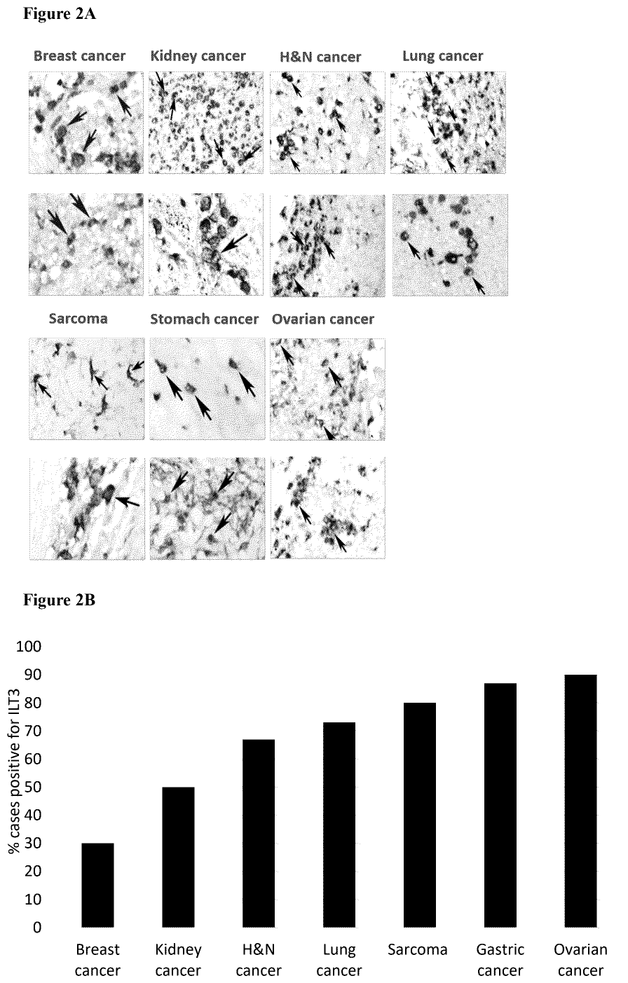

Next, immunohistochemistry was performed on sections from various tumors to check for ILT3 expression. Sections were double stained for CD68, a marker for tumor associated macrophages, and ILT3 (FIG. 2A). Samples were considered ILT3 positive if over 200 ILT3 positive cells were identified. Between 30 and 90% of breast cancers, kidney cancers, head and neck cancers, lung cancers, sarcomas, gastric cancers and ovarian cancers sampled were ILT3 positive, that is contained ILT3 positive infiltrating immune cells (FIG. 2B).

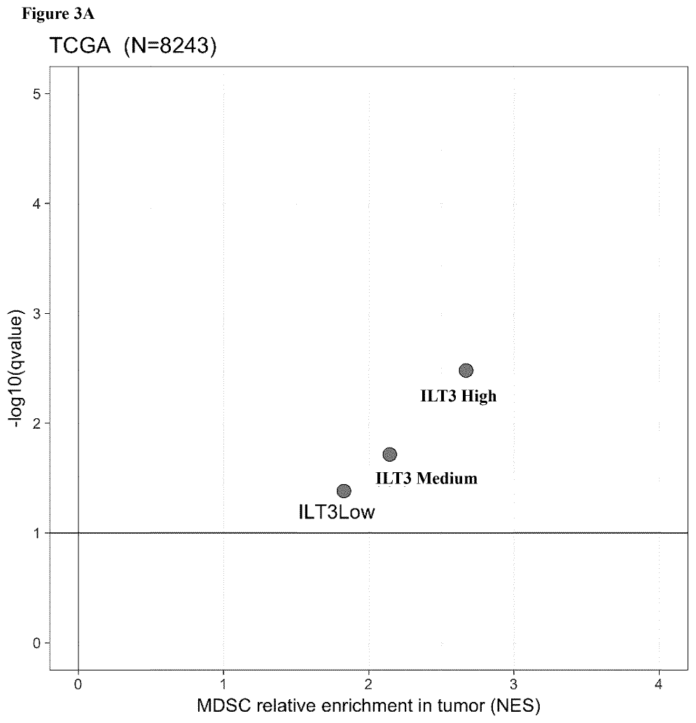

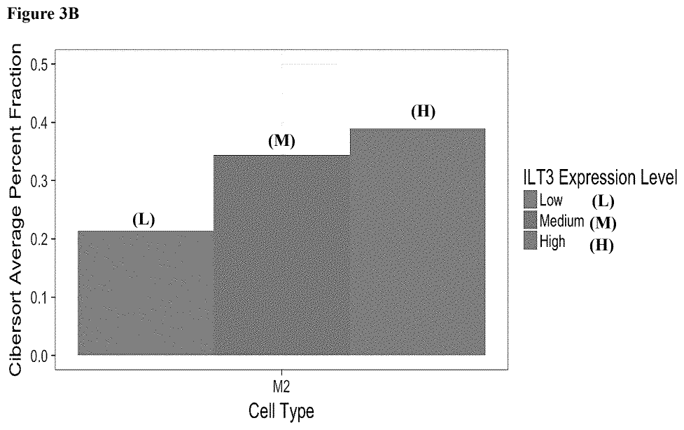

When the enrichment of MDSCs and M2 (suppressive) macrophages in cancers were compared to the average ILT3 expression in those cancers a strong correlation was found between patients with high ILT3 levels and both the enrichment of MDSCs (FIG. 3A) and M2 macrophages (FIG. 3B). All of these results emphasize the need for an antibody that can effectively block ILT3 on tumor associated immune cells and convert an immunosuppressive TME into a TME with active immune effector function.

### Example 2: Relief of Immune Cell Suppression

Anti-ILT3 antibodies were generated by hybridoma technology. The antibodies were screened for specificity to human ILT3 and for cross-reactivity to non-human primate (NHP) ILT3. Antibodies that were found to be specific to ILT3 were screened for functional activity.

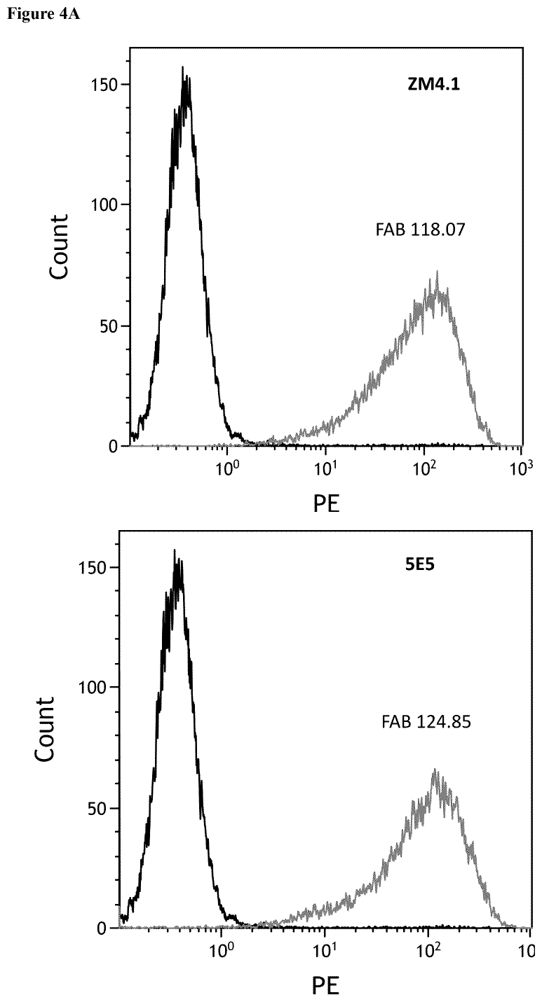

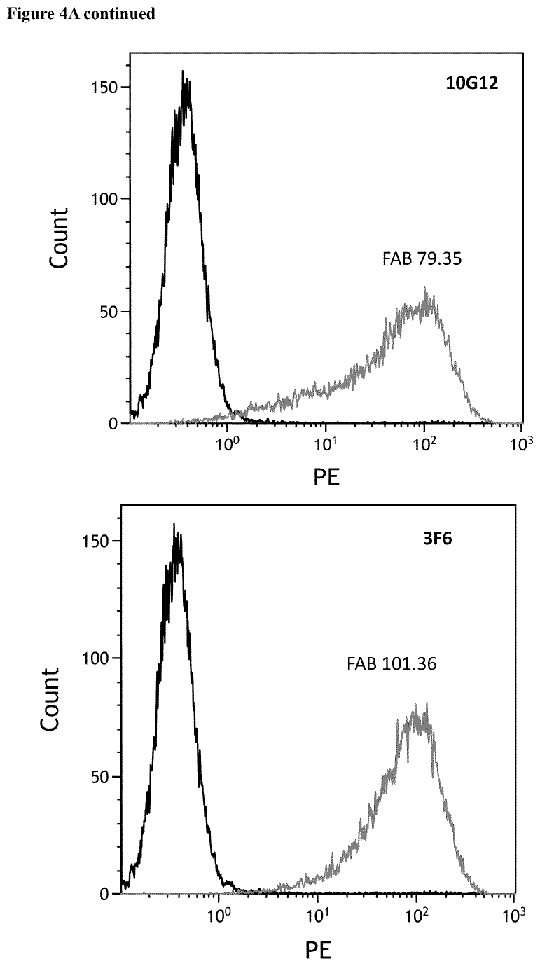

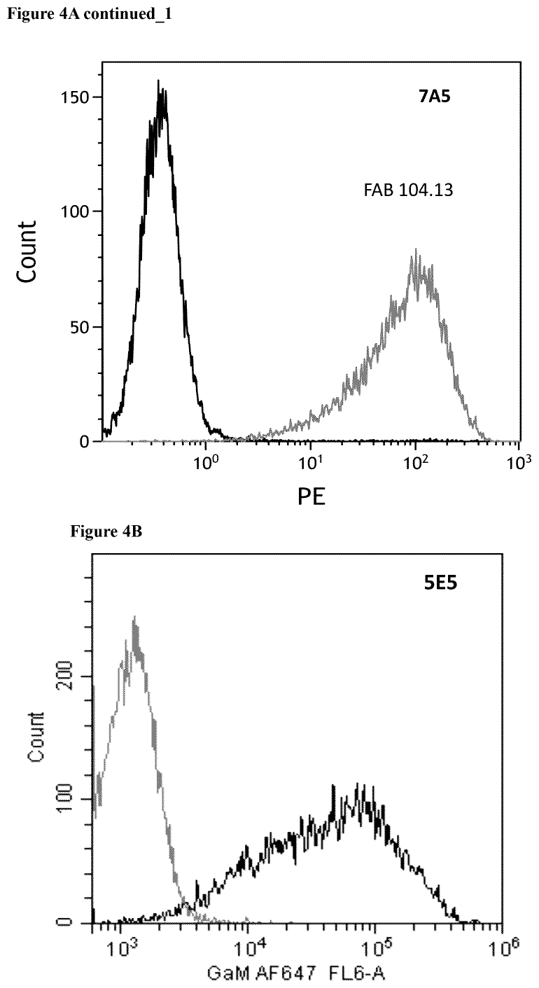

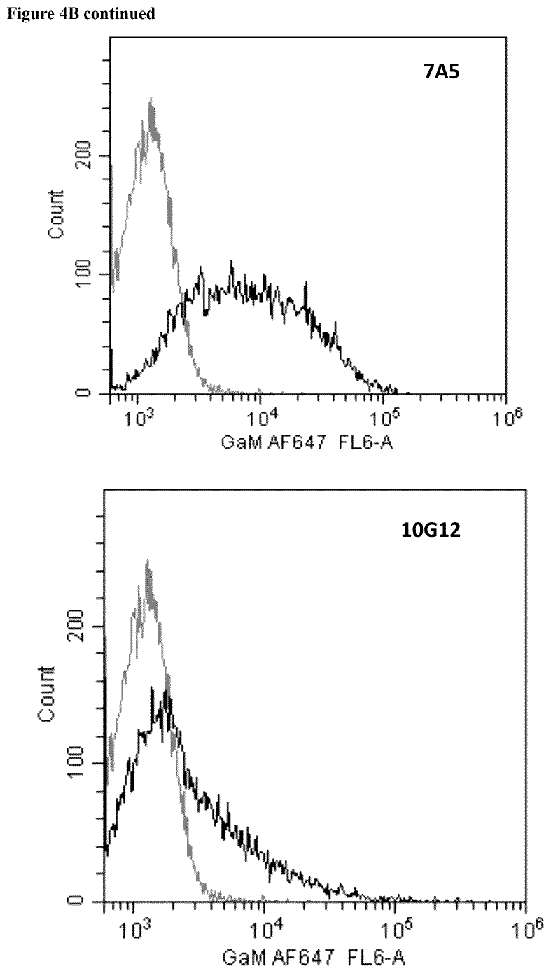

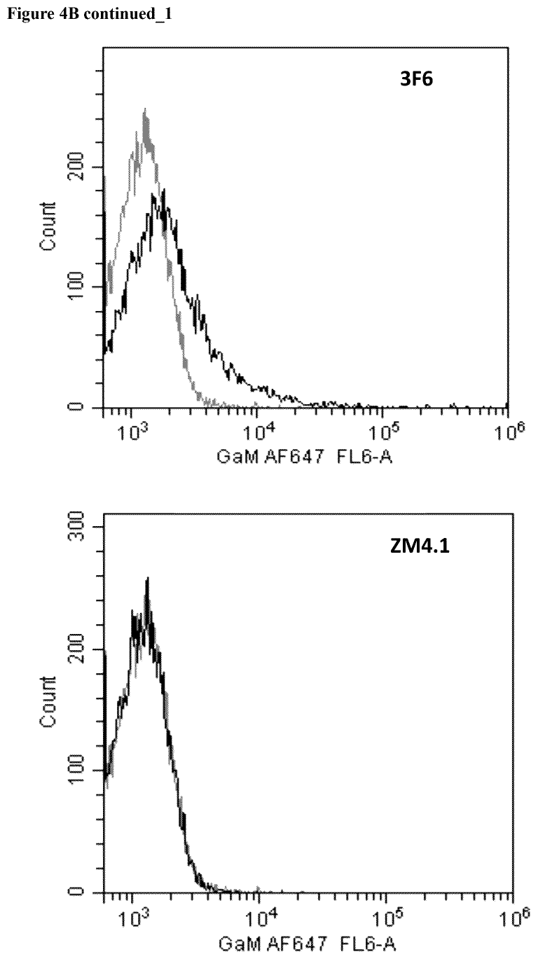

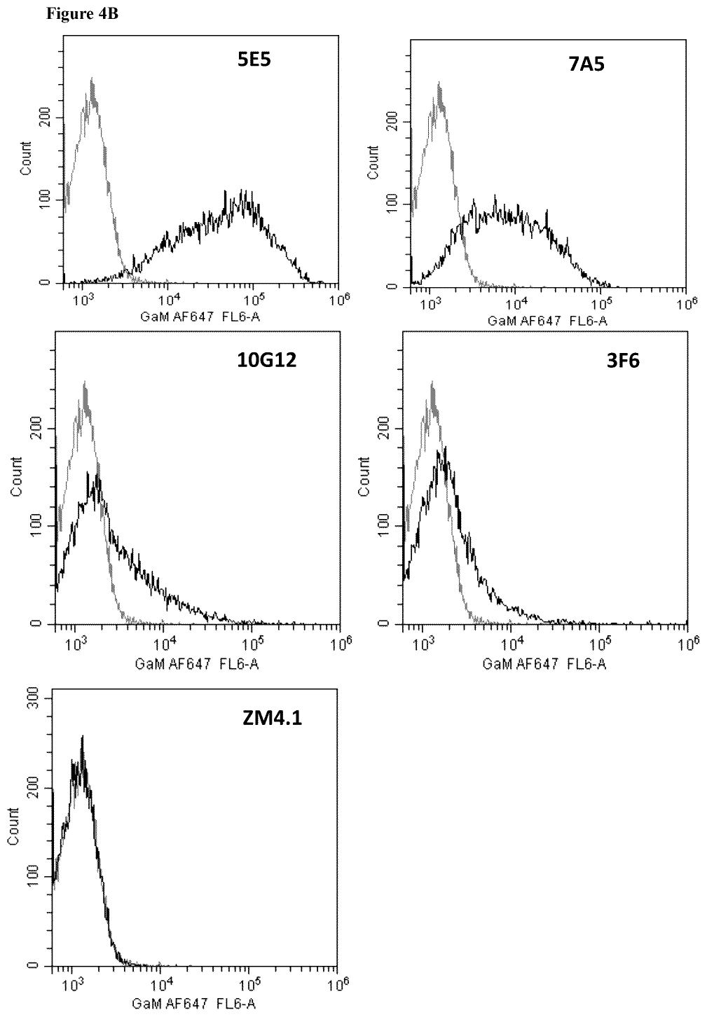

HEK cells were generated which exogenously overexpress recombinant human ILT3 and were then used for assessing antibody binding to membranal ILT3. Hybridoma media was used as a negative control and a commercial anti-ILT3 antibody (ZM4.1) was used as a positive control. The lead antibody, named 5E5, was found to produce an ILT3 expression signal that was just as good, if not better than the commercial antibody (average fluorescence 125 vs. 118 for the commercial antibody) (FIG. 4A). Three other antibodies (10G12, 3F6 and 7A5) were also found to produce positive signal and at levels that were comparable to the control antibody (10G12 showed a slightly reduced signal; 79 vs. 118). As these antibodies bound to recombinant ILT3 they are likely to also bind soluble ILT3 (SILT3). Interestingly, while 5E5 bound strongly to cynomolgus ILT3 (FIG. 4B) it did not bind mouse recombinant ILT3 at all (data not shown). The three other antibodies that were comparable to ZM4.1 also showed some binding to cynomolgus ILT3 although at noticeably lower levels than 5E5. Several other produced antibodies did not bind to cynomolgus and neither did the commercial antibody tested (ZM4.1). 5E5 was also evaluated for binding to other ILT proteins by ELISA and was not found to bind ILT5, ILT11 or ILT8, though several of the other antibodies produced from the hybridoma did show cross-reactivity.

A competition assay was performed to examine if the 5E5 antibody binds the same epitope as the commercial antibody ZM4.1. The ILT3 antibodies were biotinylated and used at a constant concentration in an ILT3 binding ELISA (1 μg/mL). Competing antibodies were added at varying dilutions and OD was measured. As a positive control, a “naked” antibody identical to the biotinylated antibody was added using the same range of concentrations. 5E5 and ZM4.1 did not compete with each other, indicating that 5E5 binds a unique epitope (FIG. 5). Interestingly, another of the hybridoma antibodies (AB #2) did compete with ZM4.1.

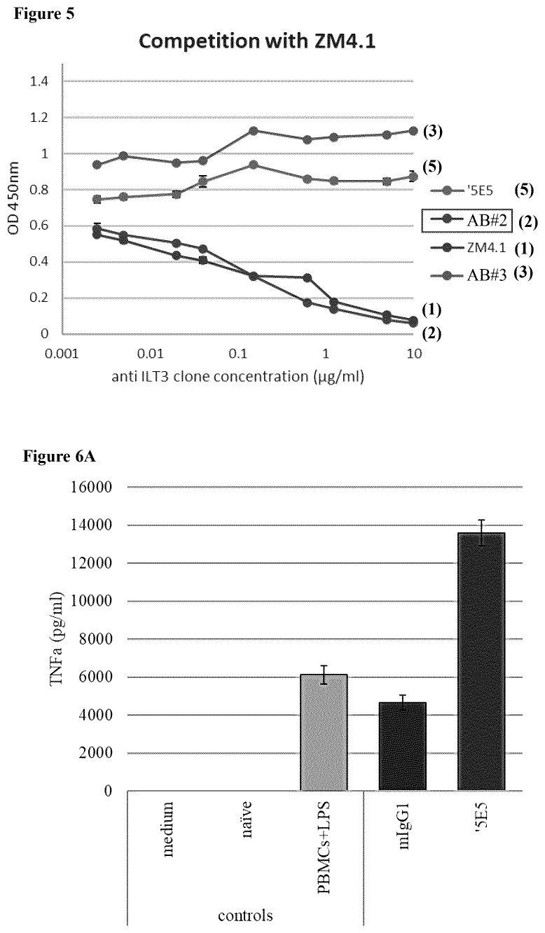

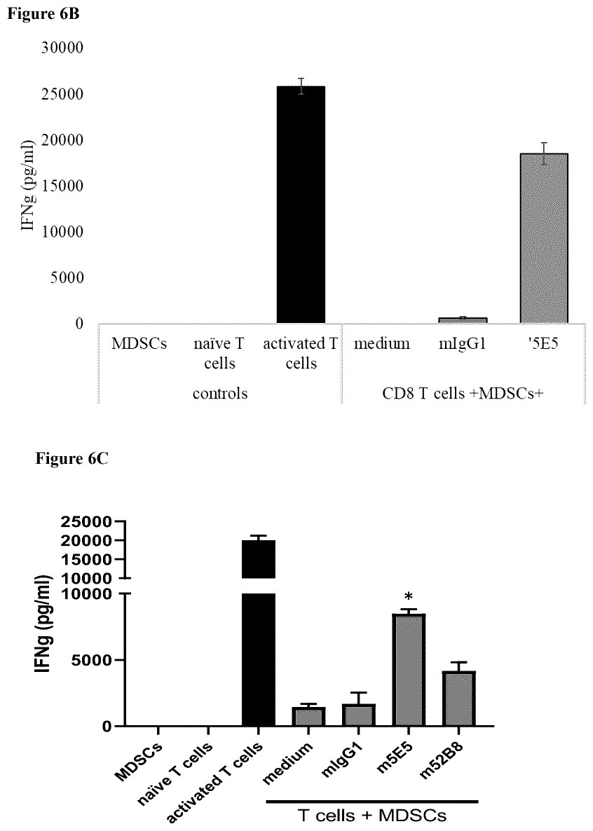

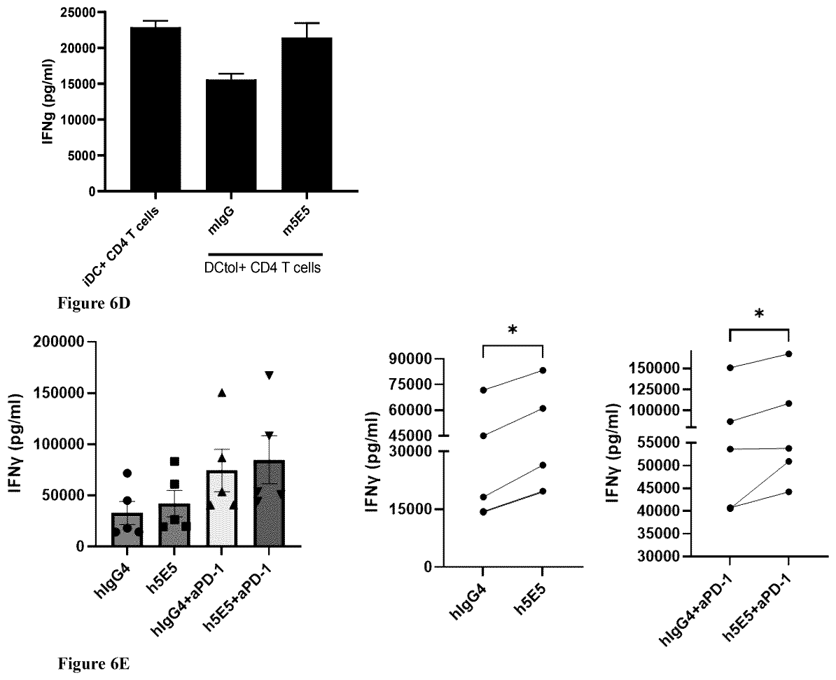

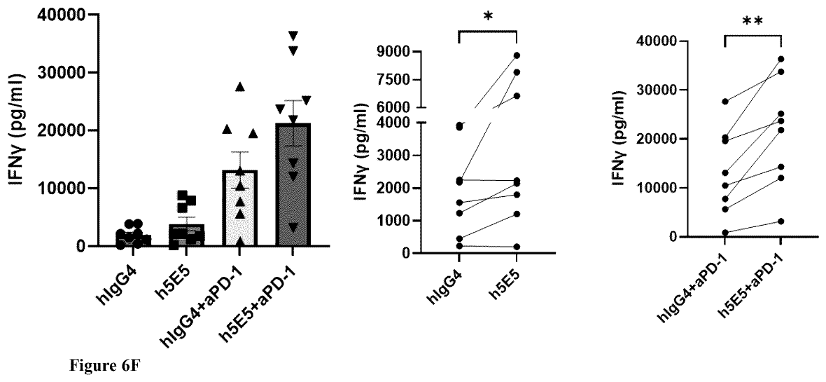

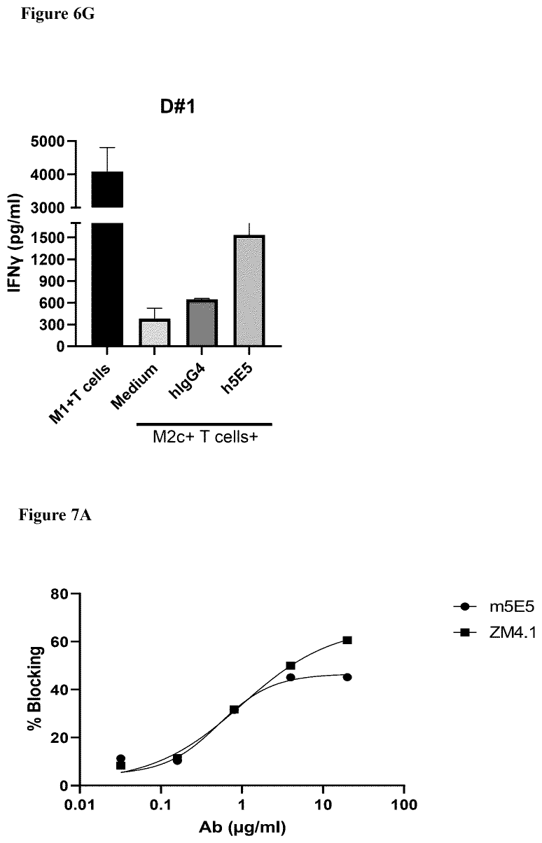

Next, the 5E5 antibody was evaluated for its ability to enhance PBMC activation. PBMCs were isolated form healthy donors and activated with LPS in the presence or absence of the 5E5 antibody for 6 hours at 37° C. TNF-α secretion levels were quantified by ELISA as a measure of immune cell activation. Medium alone and naïve PBMCs were used as negative controls, as was a mouse IgG1. 5E5 was able to more than double the levels of TNF-α secretion as compared to LPS stimulation without the antibody (FIG. 6A) indicating that this antibody can indeed enhance immune cell activation. Notably, several of the other generated antibodies, though specific to ILT3 did not enhance activation (data not shown).

T cell activation was examined next. MDSCs were generated from PBMCs from healthy donors as described hereinabove. Autologous CD8 T cells were activated with anti-CD3/CD28 beads. The activated T cells were cocultured with the MDSCs in the presence or absence of the 5E5 antibody and interferon gamma (IFNg) was measured. As can be seen in FIG. 6B, activated T cells alone secreted very high levels of IFNg which was essentially completely abolished by coculture with MDSCs (medium or mIgG1 control). However, the 5E5 antibody nearly completely abolished the inhibitory effect of the MDSCs, keeping IFNg levels at greater than 70% of those observed from activated T cells alone.

The 5E5 antibody was now compared with m52B8 antibody (Merck). While the 52B8 did restore some IFNg secretion indicating blocking of the MDSC's effect, the 5E5 antibody was found to be superior (FIG. 6C). The level of IFNg increase as compared to the negative controls (medium/mIgG1) was nearly twice as great with the 5E5 antibody as compared to the m52B8 antibody.

iDCs were generated from healthy donor monocytes and converted to DCtol as described hereinabove. Allogeneic CD4 T cells were activated with soluble anti-CD3 antibody and then incubated with the DCtol in the presence of 5E5 or mIgG1 control. CD4 T cells were also incubated with iDCs as a positive control. As can be seen in FIG. 6D, the DCtols reduced IFNg secretion from the T cells, but levels were restored to those of the control (iDCs) when 5E5 antibody was added. Thus, the antibody is capable of completely abolishing the immunosuppressive effect of DCtols on CD4 T cells. This experiment was repeated with iDCs generated for 5 different healthy donors and their allogeneic CD4 T cells. Once again, the increase in IFNg secretion produced by the 5E5 antibody was observed and was found to be statistically significant (FIG. 6E). The experiment was also run in combination with anti-PD-1 checkpoint blockade. As would be expected, the anti-PD-1 antibody administered alone (with an isotype control antibody) produced robust IFNg secretion and interestingly the h5E5 antibody produced an additive effect on top of the PD-1 effect, indicating that the two work through different pathways. This additive increase was also statistically significant. When the T cells were not first activated with anti-CD3 antibody similar results were observed though the overall IFNg secretion was much lower (FIG. 6F). Unexpectedly, without anti-CD3 activation, the combination of anti-PD-1 and 5E5 no longer produced merely an additive increase, but rather a synergistic increase was achieved with the IFNg secretion level increasing by much more than would be expected based on the effect that the 5E5 antibody had on its own.

In a parallel experiment, M2c macrophages were generated from monocytes isolated from healthy donors. Briefly, monocytes were cultured in complete RPMI medium supplemented with M-CSF (50 ng/ml) for 5-6 days to generated M-0 macrophages. M-0 macrophages were stimulated for 48 hours with IL10 (20 ng/ml) to generate M2c macrophages. The M2c macrophages were then incubated with allogenic CD4 T cells for 120 hours and levels of IFNg secretion were measured by ELISA. As expected, M2c macrophages greatly inhibited IFNg secretion from the T cells. However, when the M2c generation protocol was performed in the presence of 5E5 the M2c cells were less inhibitory, with the T cells producing higher levels of IFNg (FIG. 6G). The isotype control was similar to the results produced when no antibody was added and M1 macrophages were used as a positive control.

### Example 3: ILT3 Ligand Blocking

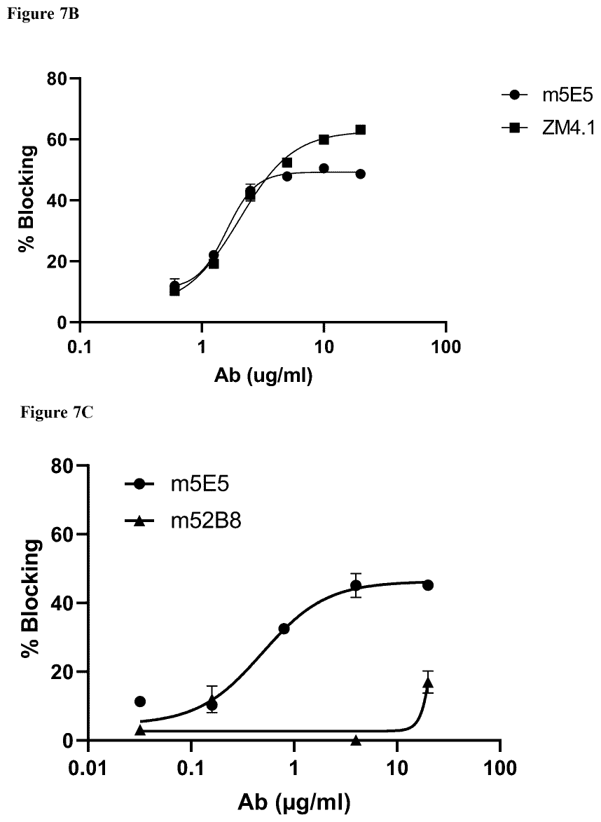

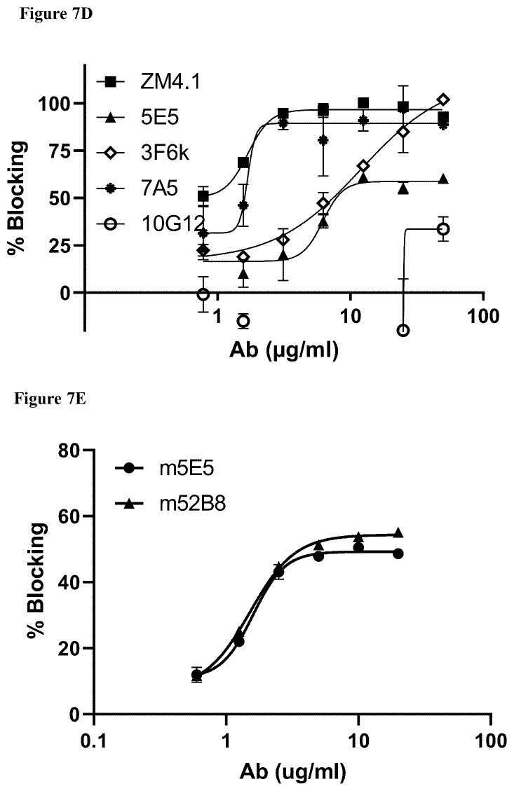

In order to further characterize the 5E5 antibody, its ability to block the binding of known ILT3 ligands fibronectin (FN1) and Apolipoprotein E (APOE) to ILT3 was evaluated. The binding of recombinant ILT3-His to fibronectin or APOE was determined by ELISA using anti-His conjugated to HRP by O.D analysis. The percent of blocking was determined by normalizing to the control (ILT3 binding without anti-ILT3 antibodies). 5E5 was compared to both ZM4.1 and m52B8. ZM4.1 was able to inhibit the binding of both ligands, and 5E5 was found to be comparable with respect to FN1 (FIG. 7A) and APOE (FIG. 7B). 5E5 produced a greater level of blocking at equal concentration. m52B8 was found to be a very poor blocker of ILT3-FN1 interaction, producing no blocking except at the highest concentration tested, whereas 5E5 was clearly superior (FIG. 7C). Several of the other antibodies produced also blocked FN1 binding, though once again 10G12 showed slightly inferior function (FIG. 7D). The two antibodies were nearly identical with respect to blocking of APOE, although the m52B8 antibody may have been slightly better (FIG. 7E).

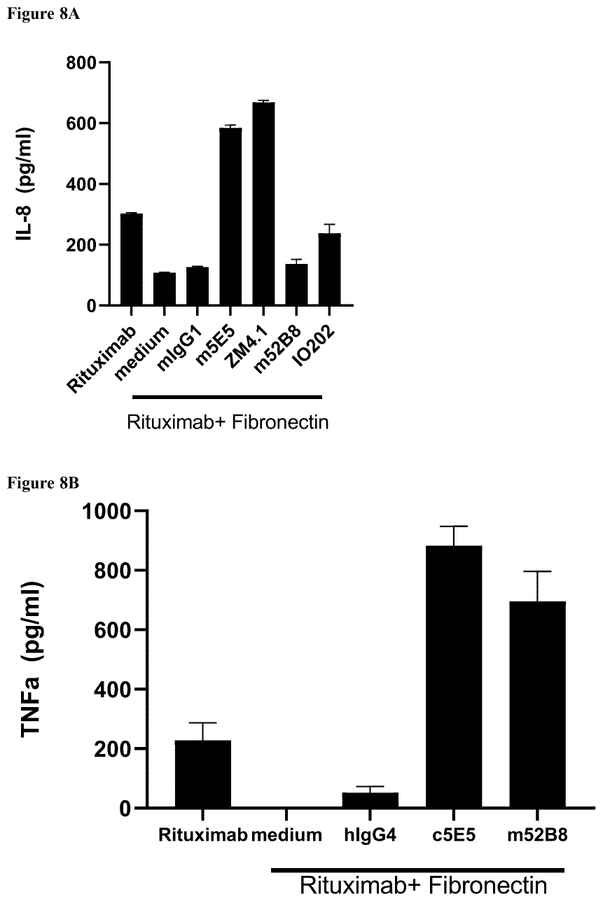

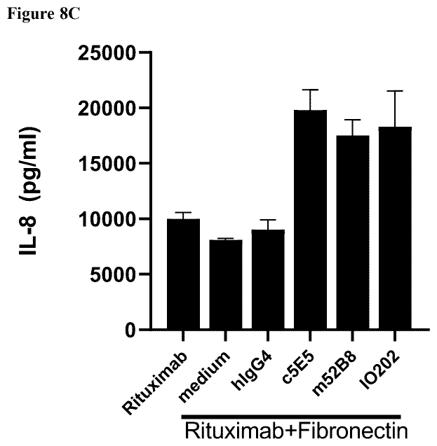

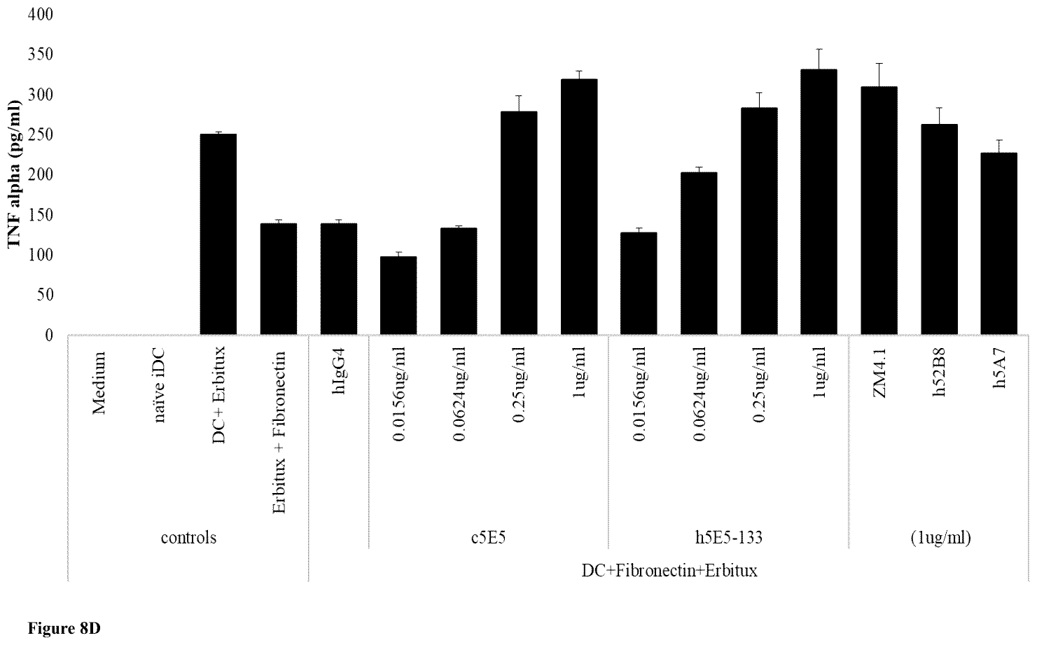

In order to functionally test the blocking of ligand binding to ILT3, THP-1 monocyte cells were activated with immobilized Rituximab through the Fc receptor signaling pathway. Activation was monitored by measuring proinflammatory cytokine IL-8 secretion. When fibronectin was added to the culture it inhibited IL-8 secretion, decreasing it by ˜67% (FIG. 8A). Various anti-ILT3 antibodies were then tested for their ability to restore IL-8 secretion. As expected, m52B8 had no effect on IL-8 secretion as it was not able to block fibronectin binding to ILT3. Antibody IO202 (Immune-One Therapeutics) had a modest effect, increasing IL-8 secretion but not restoring levels to those observed in absence of fibronectin. In contrast, 5E5 and ZM4.1 both not only restored IL-8 levels but actually increased them to levels not observed with Rituximab activation alone. Both antibodies about doubled the secretion of IL-8. Similar results are observed with APOE.

A similar experiment was run with DCs. The DCs were activated via Fc receptor signaling with immobilized Rituximab. Addition of fibronectin modestly reduced secretion of TNFa (FIG. 8B) and IL-8 (FIG. 8C), which was restored by the 5E5 antibody as well as by m52B8 and IO202. ILT3 blockade increased proinflammatory cytokine secretion levels to well above those produced by Rituximab alone. Again, the increase was more than a doubling and the 5E5 was very slightly superior to these two antibodies. The same experiment was also performed using immobilized Erbitux in order to induce Fc receptor signaling mediated activation. Both the c5E5 and h5E5 antibodies (see Example 4 hereinbelow) restored TNFa secretion inhibited by fibronectin (FIG. 8D). Indeed, the antibody of the invention was slightly superior to known antibody ZM4.1 and greatly superior to a humanized version of the known m52B8 antibody (h52B8) and the known humanized h5A7 antibody (NGM Biopharmaceuticals Inc., from US Patent Application US2021/0221887). Similar results are observed with APOE instead of fibronectin.

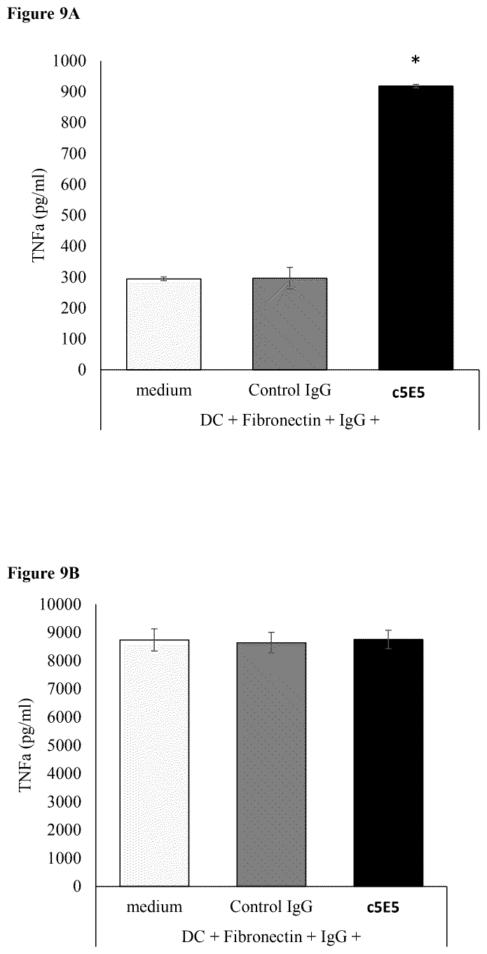

In order to demonstrate that the enhanced activation is ILT3 dependent, ILT3 expressing and non-expressing DCs were generated. The DCs were incubated with fibronectin in the presence of the 5E5 antibody of IgG control. Fibronectin only reduced TNFa secretion from the ILT3 expressing DCs (FIG. 9A) and not the non-expressing DCs (FIG. 9B). Similar results are observed with APOE. The 5E5 antibody had no effect on the non-expressing DCs and partially restored TNFa secretion from the ILT3 positive DCs. Thus, the inhibition and restoration of proinflammatory cytokine secretion is clearly ILT3 dependent.

### Example 4: Generating and Validating a Humanized 5E5 Antibody

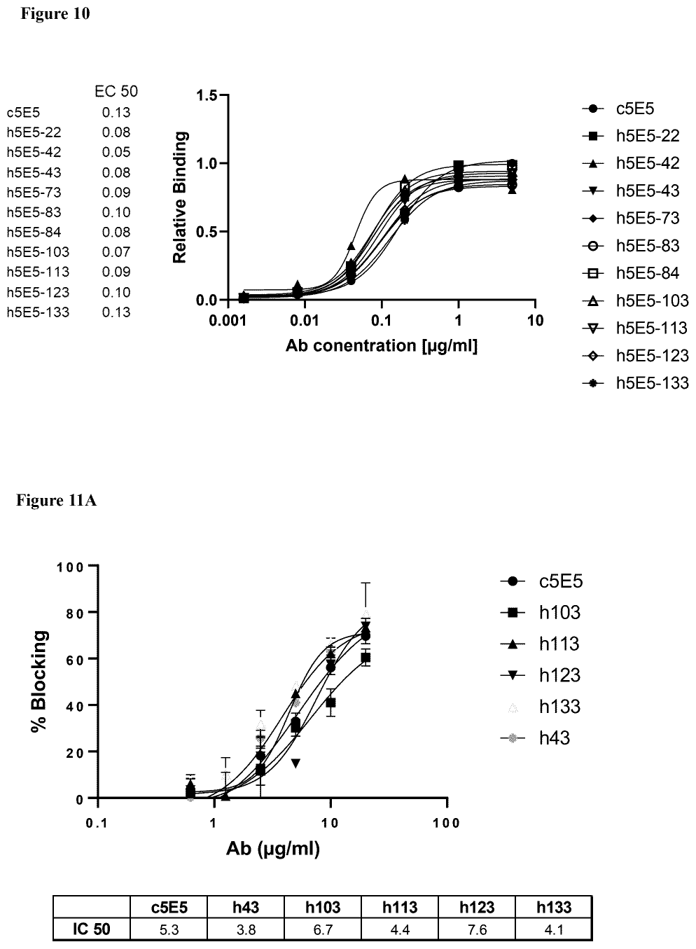

In order to generate a humanized 5E5 antibody, the mouse antibody (heavy chain SEQ ID NO: 7, light chain SEQ ID NO: 8) was sequenced and the CDRs (SEQ ID NO: 15-17 and 4-6 by the Chothia numbering system or SEQ ID NO: 13, 32, 17 and 4-6 by the Kabat numbering system) identified. The variable region of the heavy and light chain was transferred to a human constant region to produce a chimeric antibody (c5E5, heavy chain SEQ ID NO: 9, light chain SEQ ID NO: 10). Additionally, the CDRs only were removed and inserted into a completely human antibody scaffold and affinity matured (h5E5). Several slightly different human antibodies were generated and examined for their ability to bind human ILT3 (FIG. 10). All the antibodies were also found to bind cynomolgus and rhesus ILT3. H5E5-133 (heavy chain SEQ ID NO: 11, light chain SEQ ID NO: 12) was determined to be the best and was used in all future experiments. The various human antibodies are summarized in Table 1.

TABLE 1
Human antibodies as identified by Sequence ID number (Chothia for CDRs)

| | CDR- | CDR- | CDR- | CDR- | Heavy | Light | Heavy | Light |
|---|---|---|---|---|---|---|---|---|
| Name | H1 | H2 | H3 | L1/2/3 | Variable | Variable | chain | chain |
| h5E5-22 | 15 | 16 | 17 | 4/5/6 | 40 | 41 | 23 | 24 |
| h5E5--42 | 20 | 16 | 17 | 4/5/6 | 42 | 41 | 25 | 24 |
| h5E5-43 | 20 | 16 | 17 | 4/5/6 | 42 | 39 | 25 | 12 |
| h5E5-73 | 15 | 16 | 18 | 4/5/6 | 43 | 39 | 26 | 12 |
| h5E5-83 | 20 | 16 | 17 | 4/5/6 | 44 | 39 | 27 | 12 |
| h5E5-84 | 20 | 16 | 17 | 4/5/6 | 44 | 45 | 27 | 28 |
| h5E5-103 | 15 | 19 | 17 | 4/5/6 | 46 | 39 | 29 | 12 |
| h5E5-113 | 20 | 19 | 17 | 4/5/6 | 47 | 39 | 30 | 12 |
| h5E5-123 | 20 | 19 | 18 | 4/5/6 | 48 | 39 | 31 | 12 |
| h5E5-133 | 20 | 19 | 17 | 4/5/6 | 38 | 39 | 11 | 12 |

The various h5E5 antibodies were confirmed to block ILT3 interaction with FN1, with the c5E5 used as a positive control (FIG. 11A). Notably, h5E5-133 was a superior blocker as compared to c5E5 (FIG. 11B).

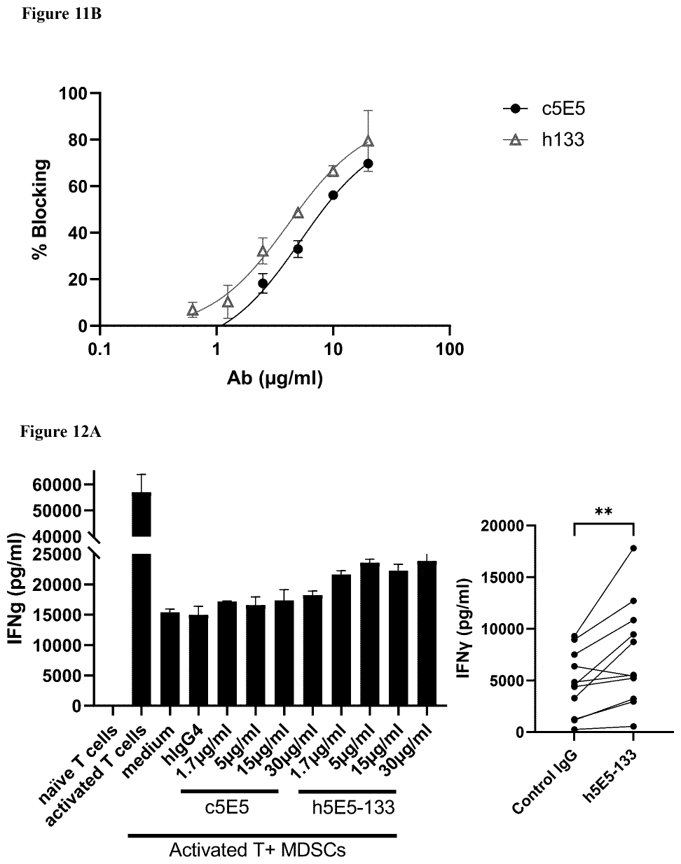

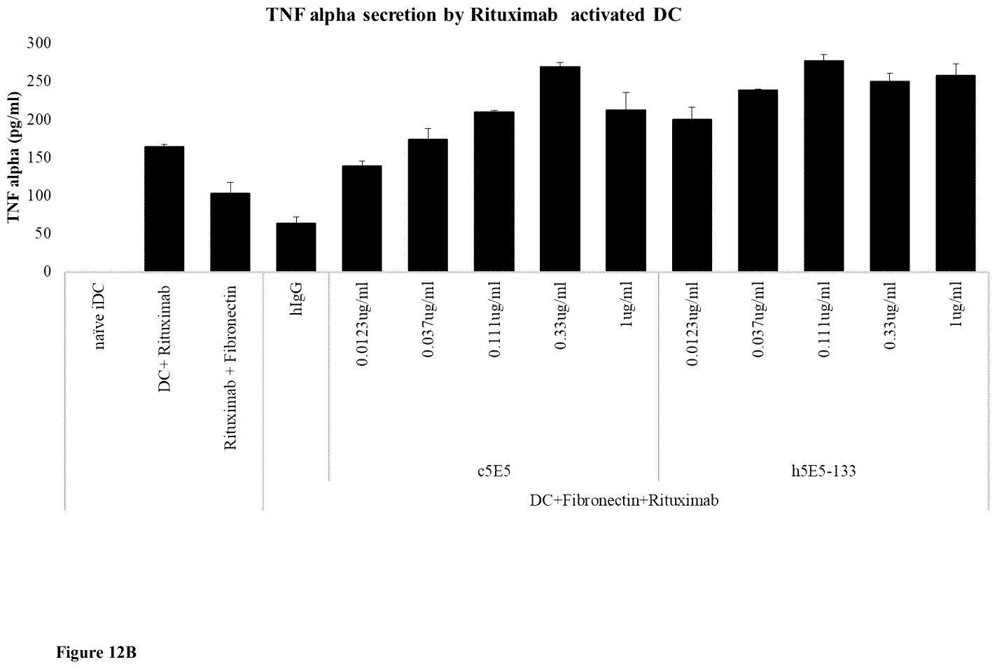

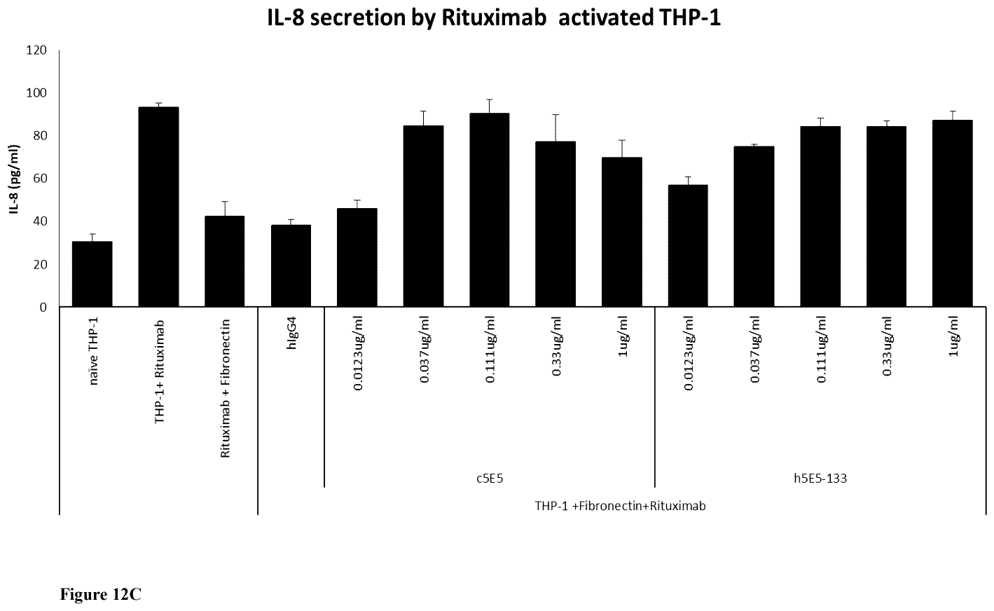

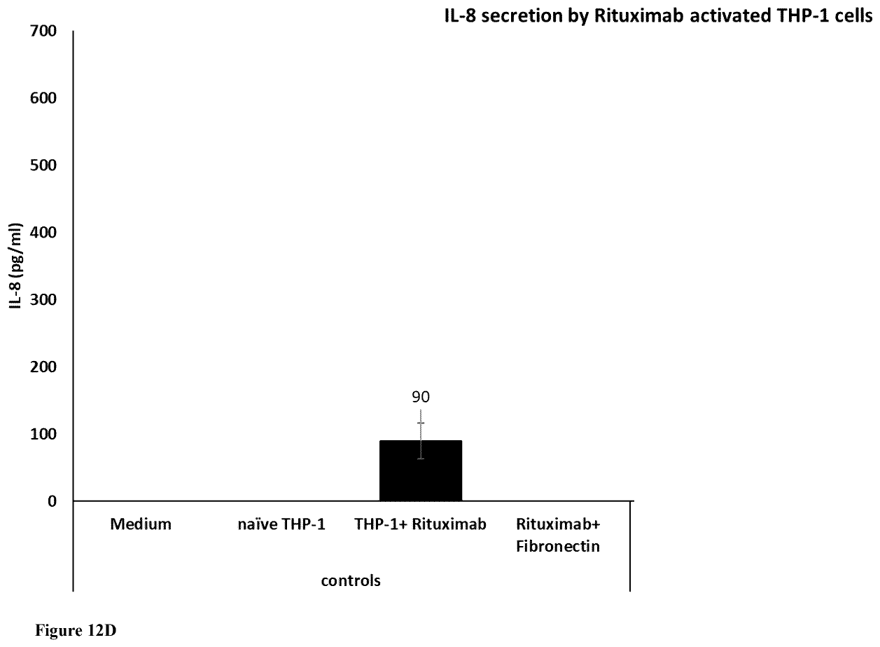

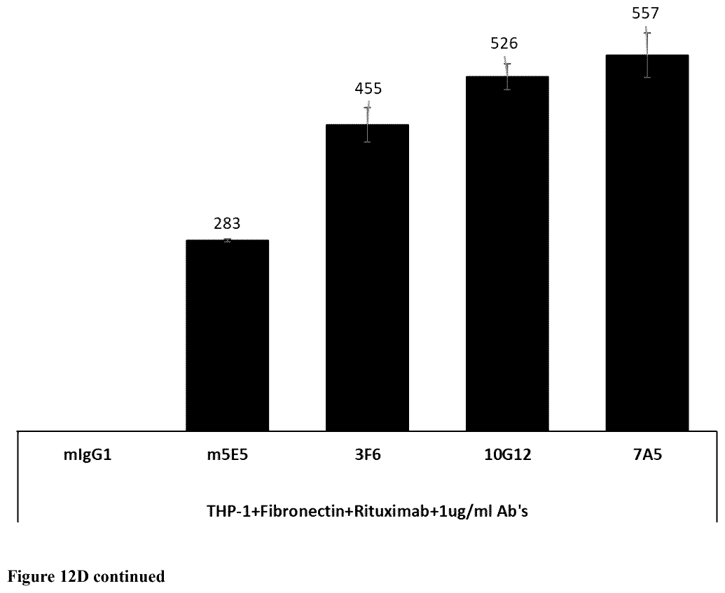

h5E5 was next examined functionally. CD8 T cell activation after MDSC suppression was examined as described hereinabove. c5E5 increased IFNg secretion from the T cells, but the h5E5 antibody was even better at alleviating MDSC suppression and produced a statistically significant increase as compared to the IgG control (FIG. 12A). DC activation in the presence of rituximab and fibronectin was examined as described hereinabove. In this assay as well h5E5-133 was again slightly superior to c5E5, having a stronger effect at lower concentrations (FIG. 12B). Similar results were also observed for restoring THP1 activation (FIG. 12C). Several other antibodies were also found to be slightly superior to the murine 5E5 (FIG. 12D). Similar results are observed with APOE instead of fibronectin.

To further evaluate h5E5 function, monocytes were stimulated with M-CSF for 5 days in the presence or absence of gastric tumor cells (GA-04) or primary colon cancer cells and the h5E5 antibody to produce M0 macrophages. The M0 macrophages then underwent the process of differentiation into M1 macrophages still in the presence or absence of the cancerous cells and h5E5 antibody and were activated by LPS stimulation. The M1 macrophages secreted high levels of TNFa, but this was abrogated when the cells were cocultured with autologous gastric cancer cells (GA-04) (FIG. 13A) or colon cells (FIG. 13B). This inhibition by the cancer cells is mediated by ILT3 as addition of h5E5 was able to relieve the inhibition and restore TNFa secretion. Addition of hIgG4 was used as a negative control. Thus, the human antibody is able to relieve cancer induced immunosuppression of M1 macrophages and emphasizes the importance of ILT3 in myeloid maturation.

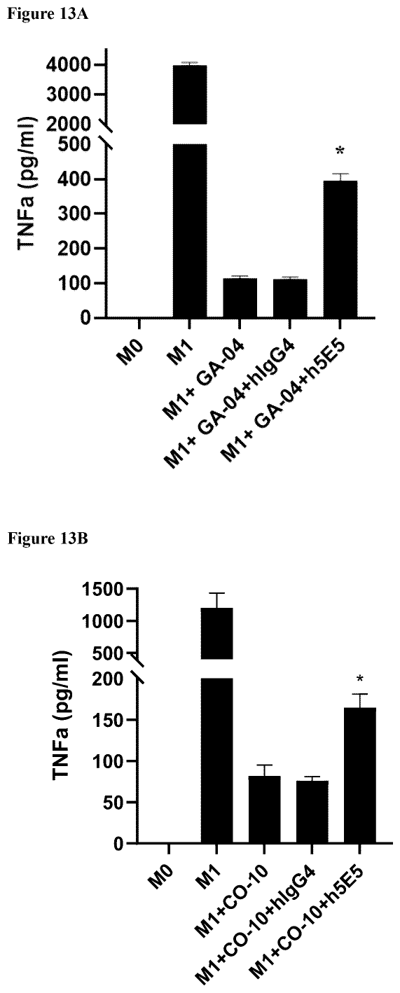

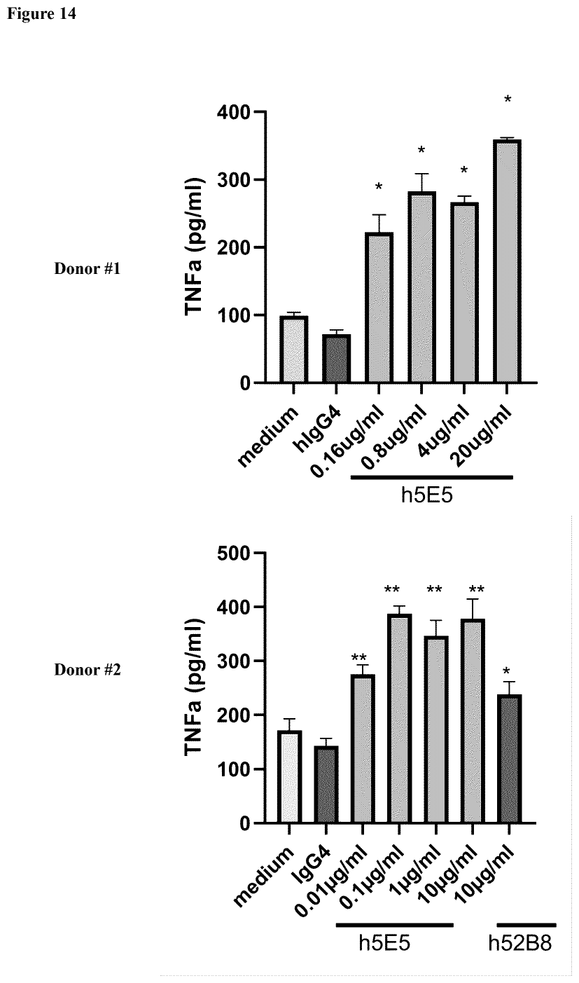

Next, monocytes were cultured in ImmunoCult DC Differentiation Medium for 5 days to generate immature DCs (iDCs). DCtol were generated by culturing iDCs with IL-10 and LPS for 2 days. The iDC stimulation was done in presence of the h5E5 antibody, the commercially available humanized h52B8 antibody, isotype control or without any additions (medium alone). TNFa secretion levels were determined by ELISA. DCtols produced very low levels of TNFa and the same was true when the isotype control was used. However, stimulation in the presence of the h5E5 antibody produced significantly increased levels of the cytokine secretion, which were superior to those produced when the h52B8 antibody was present (FIG. 14).

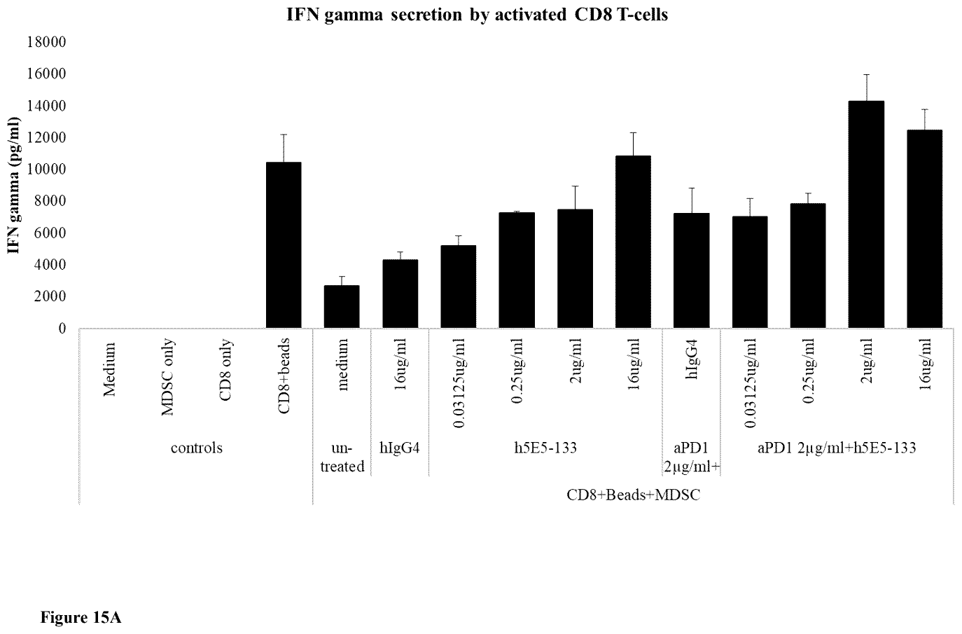

ILT3 blockade is known to be combinable with other immune checkpoint inhibitors (ICIs). To test this T cell activation in the presence of MDSCs was used to monitor the effect of h5E5, anti-PD1 and combination h5E5+PD-1 treatment. Indeed, it was found that the two blockades have a combined effect on T cell activation and produced a more robust effect than either antibody on its own (FIG. 15A). Indeed, the combined treatment was able to elevate IFNg secretion levels to above those observed in control activated CD8 T cells. A similar combined effect was observed when IFNg secretion from CD4 cells cocultured with DCtol was examined (FIG. 15B). Similar results were observed in the DCtols and CD4 T cell coincubation assay (FIG. 6E-6F).

### Example 5: In Vitro and In Vivo Evaluation of Therapeutic Effect of Humanized 5E5 Antibody

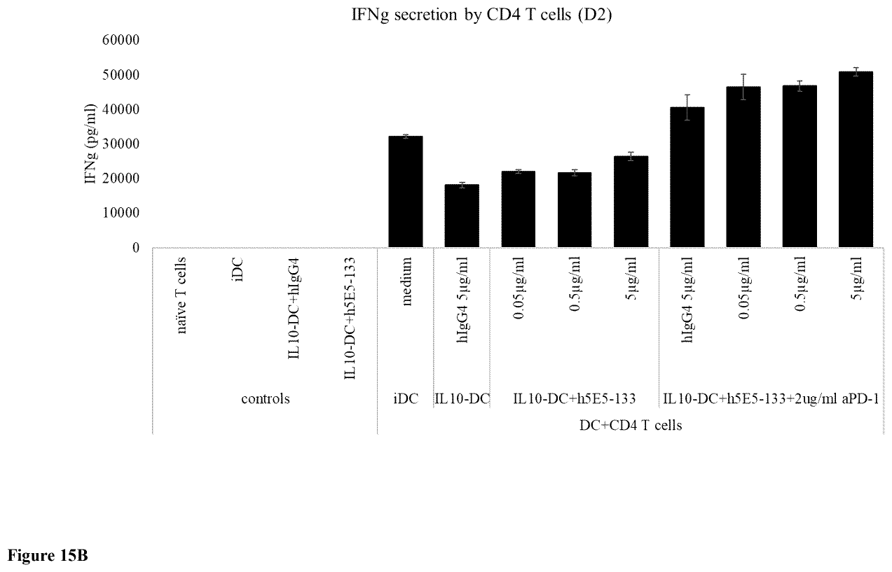

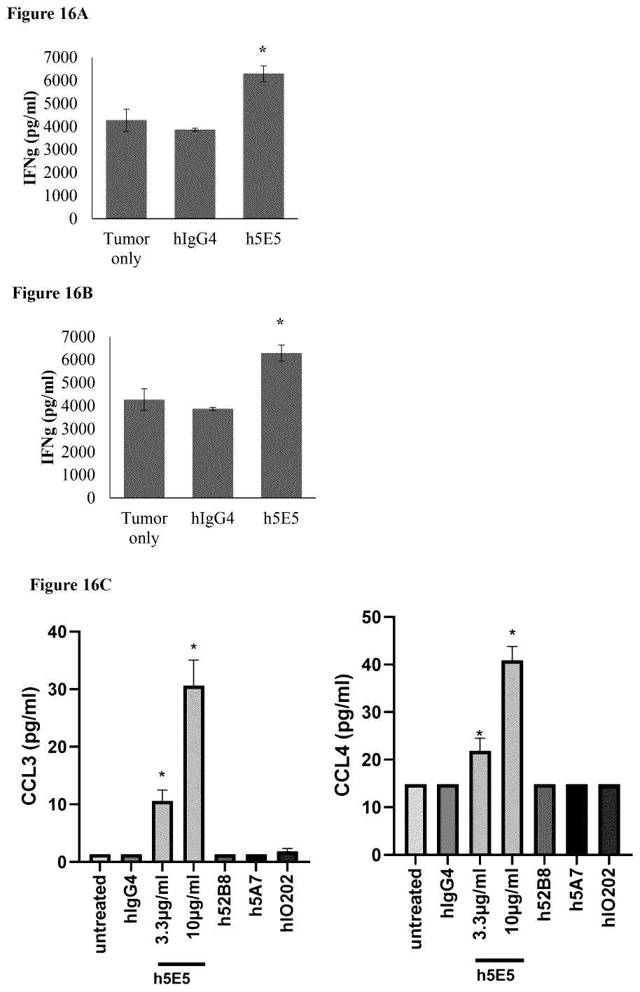

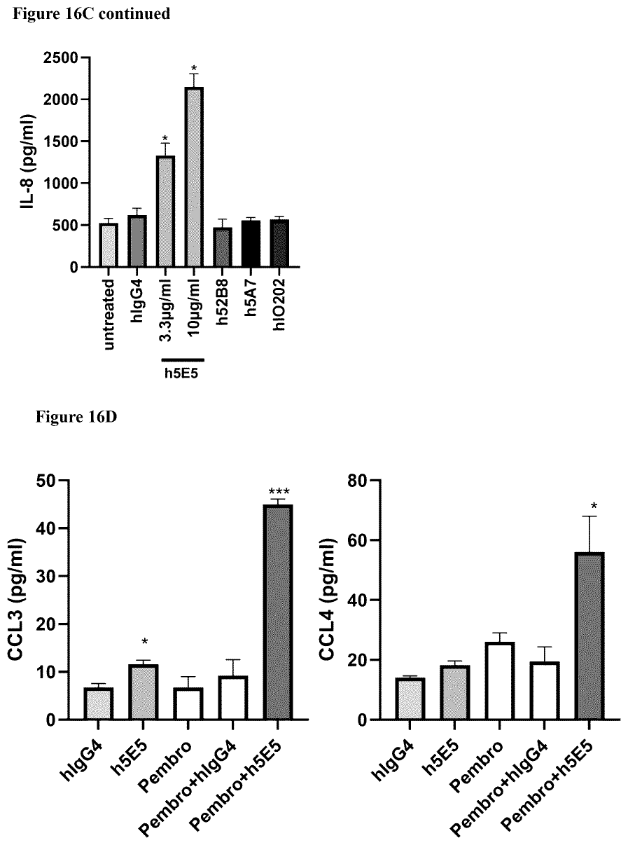

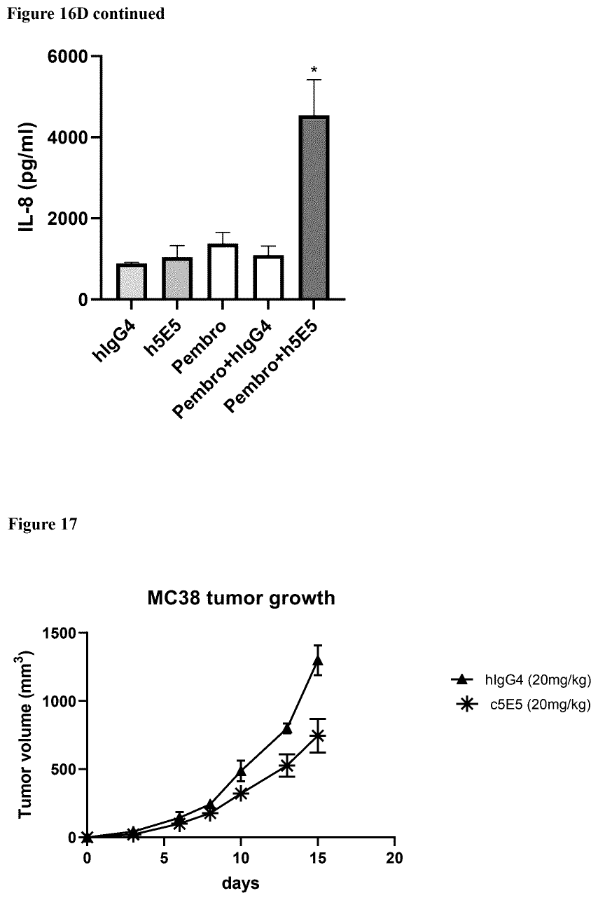

To test the therapeutic efficacy of the antibodies of the invention, primary tumor samples were processed into tumoroid particles using tissue processing by TissueGrinder. The tumoroids were activated with PMA and LPS (ionomycin was added for sarcoma tumoroids) for 48 hours and control tumoroids were unstimulated. Subsequently, the h5E5 antibody or commercially available anti-ILT3 antibodies were added, and cytokine secretion was measured by ELISA. Initial experiments found that h5E5 enhanced production of IFNg in both a sarcoma tumoroid (FIG. 16A) and a breast cancer tumoroid (FIG. 16B). When various commercially available antibodies were applied to the breast cancer tumoroid, no increase in proinflammatory chemokine (CCL3, CLL4) or cytokine (IL-8) production was observed though h5E5 produced a robust, dose-dependent increase of all tested molecules (FIG. 16C). Results in a colorectal cancer (CRC) tumoroid were similar though the effect of the h5E5 antibody was less pronounced and the h52B8 antibody produce a superior increase in cytokine/chemokine production (FIG. 16D). Surprisingly, incubation with the anti-PD-1 antibody Pembrolizumab did not have a significant effect on cytokine/chemokine secretion, however, when Pembrolizumab was combined with h5E5 the increase was highly significant and indicates a synergistic increase over each agent alone (FIG. 16D).

hILT3 transgenic mice were used to evaluate the therapeutic efficacy of the 5E5 antibody. The mice were subcutaneously inoculated with MC-38 cancer cells (a syngeneic murine colon cancer cell line). MC-38 engrafted mice were then treated with c5E5 or hIgG4 control (20 mg/kg/dose) twice a week starting from day 0 (treatments on days 0, 3, 7, 10, and 14). Tumor volume was recorded 3 times a week. As can be seen in FIG. 17, the anti-ILT3 antibody was highly effective, reducing the rate of tumor growth and the overall size of the tumor. Thus, the antibody of the invention is an effective therapeutic agent for treating cancer. Mice inoculated with tumor cells that moderately or poorly respond to anti-PD-1 therapy are also administered pembrolizumab alone or in combination with h5E5 and a synergistic reduction in tumor size is observed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An antibody or antigen binding fragment thereof comprising three heavy chain CDRs (CDR-H) and three light chain CDRs (CDR-L), wherein:
CDR-H1 comprises the amino acid sequence set forth in SEQ ID NO: 1 (GYSFX₁GF) wherein X₁ is S or T, CDR-H2 comprises the amino acid sequence as set forth in SEQ ID NO: 2 (FPSX₂GE) wherein X₂ is S or N, CDR-H3 comprises the amino acid sequence as set forth in SEQ ID NO: 3 (QAFYYFDX₃) wherein X₃ is S or Y, CDR-L1 comprises the amino acid sequence as set forth in SEQ ID NO: 4 (KSSQSLLSSSNQKNYLA), CDR-L2 comprises the amino acid sequence as set forth in SEQ ID NO: 5 (WASTRES), and CDR-L3 comprises the amino acid sequence as set forth in SEQ ID NO: 6 (QQYYSYPLT).

2. The antibody of antigen binding fragment of claim 1, wherein:
a. SEQ ID NO: 1 is SEQ ID NO: 15 (GYSFTGF), SEQ ID
NO: 2 is SEQ ID NO: 16 (FPSNGE) and SEQ ID NO: 3
is SEQ ID NO: 17 (QAFYYFDY);
b. SEQ ID NO: 1 is SEQ ID NO: 15 (GYSFTGF), SEQ ID
NO: 2 is SEQ IDNO: 16 (FPSNGE) and SEQ ID NO: 3
is SEQ ID NO: 18 (QAFYYFDS);
c. SEQ ID NO: 1 is SEQ ID NO: 15 (GYSFTGF), SEQ ID
NO: 2 is SEQ ID NO: 19 (FPSSGE) and SEQ ID NO: 3
is SEQ ID NO: 17 (QAFYYFDY);
d. SEQ ID NO: 1 is SEQ ID NO: 20 (GYSFSGF), SEQ ID
NO: 2 is SEQ ID NO: 16 (FPSNGE) and SEQ ID NO: 3
is SEQ ID NO: 17 (QAFYYFDY);
e. SEQ ID NO: 1 is SEQ ID NO: 20 (GYSFSGF), SEQ ID
NO: 2 is SEQ ID NO: 19 (FPSSGE) and SEQ ID NO: 3
is SEQ ID NO: 17 (QAFYYFDY);
or
f. SEQ ID NO: 1 is SEQ ID NO: 20 (GYSFSGF), SEQ ID
NO: 2 is SEQ ID NO: 19 (FPSSGE) and SEQ ID NO: 3
is SEQ ID NO: 18 (QAFYYFDS).

3. The antibody or antigen binding fragment of claim 2, comprising at least one of
a. a heavy chain variable region comprising the amino acid sequence of SEQ ID NO: 34 and a light chain variable region comprising the amino acid sequence of SEQ ID NO: 35;
b. a heavy chain variable region comprising the amino acid sequence of SEQ ID NO: 36 and a light chain variable region comprising the amino acid sequence of SEQ ID NO: 37;
c. a heavy chain variable region comprising the amino acid sequence of SEQ ID NO: 38 and a light chain variable region comprising the amino acid sequence of SEQ ID NO: 39;
d. a heavy chain variable region comprising the amino acid sequence of SEQ ID NO: 40 and a light chain variable region comprising the amino acid sequence of SEQ ID NO: 41;
e. a heavy chain variable region comprising the amino acid sequence of SEQ ID NO: 42 and a light chain variable region comprising the amino acid sequence of SEQ ID NO: 41;
f. a heavy chain variable region comprising the amino acid sequence of SEQ ID NO: 42 and a light chain variable region comprising the amino acid sequence of SEQ ID NO: 39;
g. a heavy chain variable region comprising the amino acid sequence of SEQ ID NO: 43 and a light chain variable region comprising the amino acid sequence of SEQ ID NO: 39;
h. a heavy chain variable region comprising the amino acid sequence of SEQ ID NO: 44 and a light chain variable region comprising the amino acid sequence of SEQ ID NO: 45;
i. a heavy chain variable region comprising the amino acid sequence of SEQ ID NO: 44 and a light chain variable region comprising the amino acid sequence of SEQ ID NO: 39;
j. a heavy chain variable region comprising the amino acid sequence of SEQ ID NO: 46 and a light chain variable region comprising the amino acid sequence of SEQ ID NO: 39;
k. a heavy chain variable region comprising the amino acid sequence of SEQ ID NO: 47 and a light chain variable region comprising the amino acid sequence of SEQ ID NO: 39; and
l. a heavy chain variable region comprising the amino acid sequence of SEQ ID NO: 48 and a light chain variable region comprising the amino acid sequence of SEQ ID NO: 39.

4. An antibody or antigen binding fragment thereof comprising three heavy chain CDRs (CDR-H) and three light chain CDRs (CDR-L), wherein:
CDR-H1 comprises the amino acid sequence set forth in SEQ ID NO: 49 (SYAMS), CDR-H2 comprises the amino acid sequence as set forth in SEQ ID NO: 50 (AITFGGGNTYYPDSVKG), CDR-H3 comprises the amino acid sequence as set forth in SEQ ID NO: 51 (HGDGNYDFYAMDY), CDR-L1 comprises the amino acid sequence as set forth in SEQ ID NO: 52 (KSSQSLLNSGNQKNYLT), CDR-L2 comprises the amino acid sequence as set forth in SEQ ID NO: 5 (WASTRES), and CDR-L3 comprises the amino acid sequence as set forth in SEQ ID NO: 53 (QNDYSYPLT);
CDR-H1 comprises the amino acid sequence set forth in SEQ ID NO: 49 (SYAMS), CDR-H2 comprises the amino acid sequence as set forth in SEQ ID NO: 56 (TISSDGGNTYYTDSVKG), CDR-H3 comprises the amino acid sequence as set forth in SEQ ID NO: 57 (HDGRGALDY), CDR-L1 comprises the amino acid sequence as set forth in SEQ ID NO: 58 (RASQDISNYLN), CDR-L2 comprises the amino acid sequence as set forth in SEQ ID NO: 59 (YTSRLHS), and CDR-L3 comprises the amino acid sequence as set forth in SEQ ID NO: 60 (QQGNTLPWT); or
CDR-H1 comprises the amino acid sequence set forth in SEQ ID NO: 63 (NSAVH), CDR-H2 comprises the amino acid sequence as set forth in SEQ ID NO: 64 (VIWAGGNTNYNSTLMS), CDR-H3 comprises the amino acid sequence as set forth in SEQ ID NO: 65 (HETYGDSFDY), CDR-L1 comprises the amino acid sequence as set forth in SEQ ID NO: 66 (RSSQSLLDSDGKTYLN), CDR-L2 comprises the amino acid sequence as set forth in SEQ ID NO: 67 (LVSKLDS), and CDR-L3 comprises the amino acid sequence as set forth in SEQ ID NO: 68 (WQGTHFPFT).

5. The antibody or antigen binding fragment of claim 4, comprising
a. a heavy chain variable region comprising the amino acid sequence of SEQ ID NO: 54 and a light chain variable region comprising the amino acid sequence of SEQ ID NO: 55;
b. a heavy chain variable region comprising the amino acid sequence of SEQ ID NO: 61 and a light chain variable region comprising the amino acid sequence of SEQ ID NO: 62; or
c. a heavy chain variable region comprising the amino acid sequence of SEQ ID NO: 69 and a light chain variable region comprising the amino acid sequence of SEQ ID NO: 70.

6. (canceled)

7. (canceled)

8. (canceled)

9. (canceled)

10. The antibody of antigen binding fragment of claim 2, wherein said heavy chain comprises an IgG4 constant region, optionally wherein said IgG4 constant region comprises a sequence with at least 80% sequence identity to SEQ ID NO: 21 (ASTKGPSVFPLAPCSRSTSESTAALGCLVKDYFPEPVTVSWNSGALTSGV HTFPAVLQSSGLYSLSSVVTVPSSSLGTKTYTCNVDHKPSNTKVDKRVES KYGPPCPPCPAPEFEGGPSVFLFSPKPKDTLMISRTPEVTCVVVDVSQEDP EVQFNWYVDGVEVHNAKTKPREEQFNSTYRVVSVLTVLHQDWLNGKEY KCKVSNKGLPSSIEKTISKAKGQPREPQVYTLPPSQEEMTKNQVSLTCLVK GFYPSDIAVEWESNGQPENNYKTTPPVLDSDGSFFLYSRLTVDKSRWQEG NVFSCSVMHEALHNHYTQKSLSLSLGK), optionally wherein said SEQ ID NO: 21 comprises an S124P and an L131E mutation.

11. The antibody of antigen binding fragment of claim 2, wherein said light chain comprises a kappa constant region, optionally wherein said kappa constant region comprises a sequence with at least 80% sequence identity to SEQ ID NO: 22
(RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPVT
KSFNRGEC).

12. The antibody of antigen binding fragment of claim 11, comprising at least one of
a. a heavy chain comprising the amino acid sequence of SEQ ID NO: 7 and a light chain comprising the amino acid sequence of SEQ ID NO: 8;
b. a heavy chain comprising the amino acid sequence of SEQ ID NO: 9 and a light chain comprising the amino acid sequence of SEQ ID NO: 10;
c. a heavy chain comprising the amino acid sequence of SEQ ID NO: 11 and a light chain comprising the amino acid sequence of SEQ ID NO: 12;
d. a heavy chain comprising the amino acid sequence of SEQ ID NO: 23 and a light chain comprising the amino acid sequence of SEQ ID NO: 24;
e. a heavy chain comprising the amino acid sequence of SEQ ID NO: 25 and a light chain comprising the amino acid sequence of SEQ ID NO: 24;
f. a heavy chain comprising the amino acid sequence of SEQ ID NO: 25 and a light chain comprising the amino acid sequence of SEQ ID NO: 12;
g. a heavy chain comprising the amino acid sequence of SEQ ID NO: 26 and a light chain comprising the amino acid sequence of SEQ ID NO: 12;
h. a heavy chain comprising the amino acid sequence of SEQ ID NO: 27 and a light chain comprising the amino acid sequence of SEQ ID NO: 12;
i. a heavy chain comprising the amino acid sequence of SEQ ID NO: 27 and a light chain comprising the amino acid sequence of SEQ ID NO: 28;
j. a heavy chain comprising the amino acid sequence of SEQ ID NO: 29 and a light chain comprising the amino acid sequence of SEQ ID NO: 12;
k. a heavy chain comprising the amino acid sequence of SEQ ID NO: 30 and a light chain comprising the amino acid sequence of SEQ ID NO: 12; and
l. a heavy chain comprising the amino acid sequence of SEQ ID NO: 31 and a light chain comprising the amino acid sequence of SEQ ID NO: 12.

13. The antibody of antigen binding fragment of claim 2, wherein said antibody or antigen binding fragment is capable of binding Leukocyte immunoglobulin-like receptor subfamily B member 4 (LILRB4 or ILT3).

14. The antibody or antigen binding fragment of claim 13, wherein at least one of:
a. said ILT3 comprises the amino acid sequence provided in SEQ ID NO: 71
b. said ILT3 is on the surface of myeloid cells, dendritic cells, macrophages or a combination thereof;
c. said ILT3 is on the surface of myeloid cells and said myeloid cells are myeloid derived suppressor cells (MDSC), said dendritic cells are tolerogenic dendritic cells, said macrophages are suppressive macrophages, optionally wherein said macrophages are tumor associated macrophages (TAMs) or a combination thereof;
d. said binding to ILT3 releases T cells, monocytes, macrophages or a combination thereof from ILT3-mediated inhibition;
e. said binding to ILT3 releases T cells from ILT3-mediated inhibition and wherein release of said T cells comprises increased T cell proliferation, increased pro-inflammatory cytokine secretion by said T cells, increased pro-inflammatory chemokine secretion by said T cells or a combination thereof;
f. said binding to ILT3 releases T cells from ILT3-mediated inhibition and wherein release of said T cells comprises increased secretion of a pro-inflammatory cytokine wherein said pro-inflammatory cytokine is selected from interferon gamma (IFNG) interleukin 8 (IL-8) and tumor necrosis factor alpha (TNF-α), said pro-inflammatory chemokine is selected from C—C Motif Chemokine Ligand 3 (CCL3) and CCL4, or both;
g. said binding to ILT3 increases dendritic cell activation;
h. said binding to ILT3 increases dendritic cell activation, wherein increasing dendritic cell activation comprises restoring dendritic cell activation suppressed by FN1, APOE or both;
i. said binding to ILT3 increases dendritic cell activation, wherein increasing dendritic cell activation comprises increasing secretion of a pro-inflammatory cytokine or chemokine by said dendritic cells;
j. said binding to ILT3 increases monocyte and/or macrophage activation; and
k. said binding to ILT3 increases monocyte and/or macrophage activation, wherein increasing monocyte and/or macrophage activation comprises increasing secretion of a pro-inflammatory cytokine or chemokine by said dendritic cells.

15. (canceled)

16. The antibody or antigen binding fragment of claim 13, wherein said binding to ILT3 inhibits binding of ILT3 to an ILT3 ligand, optionally wherein said ligand is Apolipoprotein E (APOE), fibronectin (FN1) or both.

17. (canceled)

18. (canceled)

19. (canceled)

20. (canceled)

21. (canceled)

22. (canceled)

23. (canceled)

24. (canceled)

25. (canceled)

26. (canceled)

27. The antibody or antigen binding fragment of claim 2, wherein at least one of:
a. the antigen binding fragment is selected from the group consisting of a Fv, Fab, F(ab′)₂, scFV or a scFV₂ fragment;
b. said antibody or antigen binding fragment is humanized;
c. said antibody is a monoclonal antibody; and
d. said antibody or antigen binding fragment thereof does not induce antibody-dependent cell-mediated cytotoxicity (ADCC) or complement-dependent cytotoxicity (CDC).

28. (canceled)

29. (canceled)

30. (canceled)

31. A nucleic acid molecule or a plurality of nucleic acid molecules encoding an antibody or antigen binding fragment of claim 1.

32. A pharmaceutical composition comprising an antibody or antigen binding fragment of claim 1 and a pharmaceutically acceptable carrier, excipient or adjuvant.

33. The pharmaceutical composition of claim 32, formulated for administration to a subject, for systemic administration or for intratumoral administration.

34. (canceled)

35. A method of treating cancer in a subject in need thereof, the method comprising administering to said subject a pharmaceutical composition of claim 32, thereby treating cancer.

36. The method of claim 35, wherein said cancer is at least one of:
a. a solid cancer;
b. selected from breast cancer, kidney cancer, head and neck cancer, lung cancer, sarcoma, gastric cancer, colorectal cancer and ovarian cancer;
c. characterized by the presence of tumor infiltrating immune cells expressing ILT3; and
d. comprises a tumor microenvironment (TME) characterized by expression of APOE, FN1 or both.

37. (canceled)

38. (canceled)

39. (canceled)

40. (canceled)

41. The method of claim 35, further comprising administering an immune checkpoint inhibitor (ICI).

42. The method of claim 41, wherein said ICI inhibits the PD-1/PD-L1/L2 checkpoint.

43. The method of claim 42, wherein said ICI is selected from pembrolizumab, nivolumab, atezolizumab, cemiplimab, dostarlimab, durvalumab and avelumab.


SEQUENCE LISTING (71 sequences)

SEQ ID NO: 1 - protein, 7 aa, synthetic construct
  REGION 1..7: note = Synthetic
  VARIANT 5: note = X is S or T
GYSFXGF

SEQ ID NO: 2 - protein, 6 aa, synthetic construct
  REGION 1..6: note = Synthetic
  VARIANT 4: note = X is S or N
FPSXGE

SEQ ID NO: 3 - protein, 8 aa, synthetic construct
  REGION 1..8: note = Synthetic
  VARIANT 8: note = X is S or Y
QAFYYFDX

SEQ ID NO: 4 - protein, 17 aa, synthetic construct
  REGION 1..17: note = Synthetic
KSSQSLLSSSNQKNYLA

SEQ ID NO: 5 - protein, 7 aa, synthetic construct
  REGION 1..7: note = Synthetic
WASTRES

SEQ ID NO: 6 - protein, 9 aa, synthetic construct
  REGION 1..9: note = Synthetic
QQYYSYPLT

SEQ ID NO: 7 - protein, 441 aa, synthetic construct
  REGION 1..441: note = Synthetic
EVQLQQSGPELVKPGASVKISCKASGYSFTGFYIDWVKQSPGKSLEWIGYIFPSNGETSYNQKFKGKATLTVDKSSSTVNMQLNSLTSEDSAVYYCARQAFYYFDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFIYSKLNVQKSNWEAGNTFTCSVLHEGLHNHHTEKSLSHSPGK

SEQ ID NO: 8 - protein, 220 aa, synthetic construct
  REGION 1..220: note = Synthetic
DIVMSQSPSSLAVSVGEKVTMSCKSSQSLLSSSNQKNYLAWYQQKPGQSPKLLIYWASTRESGVPDRFAGSGSGTDFTLTISSVKAEDLAVYYCQQYYSYPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 444 aa, synthetic construct
  REGION 1..444: note = Synthetic
EVQLQQSGPELVKPGASVKISCKASGYSFTGFYIDWVKQSPGKSLEWIGYIFPSNGETSYNQKFKGKATLTVDKSSSTVNMQLNSLTSEDSAVYYCARQAFYYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 220 aa, synthetic construct
  REGION 1..220: note = Synthetic
DIVMSQSPSSLAVSVGEKVTMSCKSSQSLLSSSNQKNYLAWYQQKPGQSPKLLIYWASTRESGVPDRFAGSGSGTDFTLTISSVKAEDLAVYYCQQYYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 444 aa, synthetic construct
  REGION 1..444: note = Synthetic
QVQLVQSGAEVKKPGASVKVSCKASGYSFSGFYIDWVKQPPGKGLEWIGYIFPSSGETSYNQKFKGRVTMTVDKSTSTVYMELSSLRSEDTAVYYCARQAFYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 220 aa, synthetic construct
  REGION 1..220: note = Synthetic
DIVMTQSPDSLAVSLGERATINCKSSQSLLSSSNQKNYLAWYQQKPGQAPRLLIYWASTRESGVPDRFAGSGSGTDFTLTISSLQAEDVAVYYCQQYYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 5 aa, synthetic construct
  REGION 1..5: note = Synthetic
GFYID

SEQ ID NO: 14 - protein, 17 aa, synthetic construct
  REGION 1..17: note = Synthetic
  VARIANT 6: note = X is S or N
YIFPSXGETSYNQKFKG

SEQ ID NO: 15 - protein, 7 aa, synthetic construct
  REGION 1..7: note = Synthetic
GYSFTGF

SEQ ID NO: 16 - protein, 6 aa, synthetic construct
  REGION 1..6: note = Synthetic
FPSNGE

SEQ ID NO: 17 - protein, 8 aa, synthetic construct
  REGION 1..8: note = Synthetic
QAFYYFDY

SEQ ID NO: 18 - protein, 8 aa, synthetic construct
  REGION 1..8: note = Synthetic
QAFYYFDS

SEQ ID NO: 19 - protein, 6 aa, synthetic construct
  REGION 1..6: note = Synthetic
FPSSGE

SEQ ID NO: 20 - protein, 7 aa, synthetic construct
  REGION 1..7: note = Synthetic
GYSFSGF

SEQ ID NO: 21 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFSPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 107 aa, Homo sapiens [sequence omitted]

SEQ ID NO: 23 - protein, 444 aa, synthetic construct
  REGION 1..444: note = Synthetic
EVQLVQSGAEVKKPGASVKVSCKASGYSFTGFYIDWVKQPPGKGLEWIGYIFPSNGETSYNQKFKGRATMTVDKSTSTVYMELSSLRSEDTAVYYCARQAFYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 220 aa, synthetic construct
  REGION 1..220: note = Synthetic
DIVMTQSPDSLAVSLGERATINCKSSQSLLSSSNQKNYLAWYQQKPGQPPKLLIYWASTRESGVPDRFAGSGSGTDFTLTISSLQAEDVAVYYCQQYYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 444 aa, synthetic construct
  REGION 1..444: note = Synthetic
QVQLVQSGAEVKKPGASVKVSCKASGYSFSGFYIDWVKQPPGKGLEWIGYIFPSNGETSYNQKFKGRVTMTVDKSTSTVYMELSSLRSEDTAVYYCARQAFYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 444 aa, synthetic construct
  REGION 1..444: note = Synthetic
QVQLVQSGAEVKKPGASVKVSCKASGYSFTGFYIDWVKQPPGKGLEWIGYIFPSNGETSYNQKFKGRVTMTVDKSTSTVYMELSSLRSEDTAVYYCARQAFYYFDSWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 444 aa, synthetic construct
  REGION 1..444: note = Synthetic
EVQLVQSGAEVKKPGASVKVSCKASGYSFSGFYIDWVKQPPGKGLEWIGYIFPSNGETSYNQKFKGRATMTVDKSTSTVYMELSSLRSEDTAVYYCARQAFYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 220 aa, synthetic construct
  REGION 1..220: note = Synthetic
DIVMTQSPDSLAVSLGERATINCKSSQSLLSSSNQKNYLAWYQQKPGQPPKLLIYWASTRESGVPDRFSGSGSGTDFTLTISSLQAEDVAVYYCQQYYSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 444 aa, synthetic construct
  REGION 1..444: note = Synthetic
EVQLVQSGAEVKKPGASVKVSCKASGYSFTGFYIDWVKQPPGKGLEWIGYIFPSSGETSYNQKFKGRATMTVDKSTSTVYMELSSLRSEDTAVYYCARQAFYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 444 aa, synthetic construct
  REGION 1..444: note = Synthetic
EVQLVQSGAEVKKPGASVKVSCKASGYSFSGFYIDWVKQPPGKGLEWIGYIFPSSGETSYNQKFKGRATMTVDKSTSTVYMELSSLRSEDTAVYYCARQAFYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 444 aa, synthetic construct
  REGION 1..444: note = Synthetic
QVQLVQSGAEVKKPGASVKVSCKASGYSFSGFYIDWVKQPPGKGLEWIGYIFPSSGETSYNQKFKGRVTMTVDKSTSTVYMELSSLRSEDTAVYYCARQAFYYFDSWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 17 aa, synthetic construct
  REGION 1..17: note = Synthetic
YIFPSNGETSYNQKFKG

SEQ ID NO: 33 - protein, 17 aa, synthetic construct
  REGION 1..17: note = Synthetic
YIFPSSGETSYNQKFKG

SEQ ID NO: 34 - protein, 117 aa, synthetic construct
  REGION 1..117: note = Synthetic
EVQLQQSGPELVKPGASVKISCKASGYSFTGFYIDWVKQSPGKSLEWIGYIFPSNGETSYNQKFKGKATLTVDKSSSTVNMQLNSLTSEDSAVYYCARQAFYYFDYWGQGTTLTVSS

SEQ ID NO: 35 - protein, 113 aa, synthetic construct
  REGION 1..113: note = Synthetic
DIVMSQSPSSLAVSVGEKVTMSCKSSQSLLSSSNQKNYLAWYQQKPGQSPKLLIYWASTRESGVPDRFAGSGSGTDFTLTISSVKAEDLAVYYCQQYYSYPLTFGAGTKLELK

SEQ ID NO: 36 - protein, 117 aa, synthetic construct; identical to SEQ ID NO: 34
  REGION 1..117: note = Synthetic

SEQ ID NO: 37 - protein, 113 aa, synthetic construct; identical to SEQ ID NO: 35
  REGION 1..113: note = Synthetic

SEQ ID NO: 38 - protein, 117 aa, synthetic construct
  REGION 1..117: note = Synthetic
QVQLVQSGAEVKKPGASVKVSCKASGYSFSGFYIDWVKQPPGKGLEWIGYIFPSSGETSYNQKFKGRVTMTVDKSTSTVYMELSSLRSEDTAVYYCARQAFYYFDYWGQGTTVTVSS

SEQ ID NO: 39 - protein, 113 aa, synthetic construct
  REGION 1..113: note = Synthetic
DIVMTQSPDSLAVSLGERATINCKSSQSLLSSSNQKNYLAWYQQKPGQAPRLLIYWASTRESGVPDRFAGSGSGTDFTLTISSLQAEDVAVYYCQQYYSYPLTFGQGTKLEIK

SEQ ID NO: 40 - protein, 117 aa, synthetic construct
  REGION 1..117: note = Synthetic
EVQLVQSGAEVKKPGASVKVSCKASGYSFTGFYIDWVKQPPGKGLEWIGYIFPSNGETSYNQKFKGRATMTVDKSTSTVYMELSSLRSEDTAVYYCARQAFYYFDYWGQGTTVTVSS

SEQ ID NO: 41 - protein, 113 aa, synthetic construct
  REGION 1..113: note = Synthetic
DIVMTQSPDSLAVSLGERATINCKSSQSLLSSSNQKNYLAWYQQKPGQPPKLLIYWASTRESGVPDRFAGSGSGTDFTLTISSLQAEDVAVYYCQQYYSYPLTFGQGTKLEIK

SEQ ID NO: 42 - protein, 117 aa, synthetic construct
  REGION 1..117: note = Synthetic
QVQLVQSGAEVKKPGASVKVSCKASGYSFSGFYIDWVKQPPGKGLEWIGYIFPSNGETSYNQKFKGRVTMTVDKSTSTVYMELSSLRSEDTAVYYCARQAFYYFDYWGQGTTVTVSS

SEQ ID NO: 43 - protein, 117 aa, synthetic construct
  REGION 1..117: note = Synthetic
QVQLVQSGAEVKKPGASVKVSCKASGYSFTGFYIDWVKQPPGKGLEWIGYIFPSNGETSYNQKFKGRVTMTVDKSTSTVYMELSSLRSEDTAVYYCARQAFYYFDSWGQGTTVTVSS

SEQ ID NO: 44 - protein, 117 aa, synthetic construct
  REGION 1..117: note = Synthetic
EVQLVQSGAEVKKPGASVKVSCKASGYSFSGFYIDWVKQPPGKGLEWIGYIFPSNGETSYNQKFKGRATMTVDKSTSTVYMELSSLRSEDTAVYYCARQAFYYFDYWGQGTTVTVSS

SEQ ID NO: 45 - protein, 113 aa, synthetic construct
  REGION 1..113: note = Synthetic
DIVMTQSPDSLAVSLGERATINCKSSQSLLSSSNQKNYLAWYQQKPGQPPKLLIYWASTRESGVPDRFSGSGSGTDFTLTISSLQAEDVAVYYCQQYYSYPLTFGQGTKLEIK

SEQ ID NO: 46 - protein, 117 aa, synthetic construct
  REGION 1..117: note = Synthetic
EVQLVQSGAEVKKPGASVKVSCKASGYSFTGFYIDWVKQPPGKGLEWIGYIFPSSGETSYNQKFKGRATMTVDKSTSTVYMELSSLRSEDTAVYYCARQAFYYFDYWGQGTTVTVSS

SEQ ID NO: 47 - protein, 117 aa, synthetic construct
  REGION 1..117: note = Synthetic
EVQLVQSGAEVKKPGASVKVSCKASGYSFSGFYIDWVKQPPGKGLEWIGYIFPSSGETSYNQKFKGRATMTVDKSTSTVYMELSSLRSEDTAVYYCARQAFYYFDYWGQGTTVTVSS

SEQ ID NO: 48 - protein, 117 aa, synthetic construct
  REGION 1..117: note = Synthetic
QVQLVQSGAEVKKPGASVKVSCKASGYSFSGFYIDWVKQPPGKGLEWIGYIFPSSGETSYNQKFKGRVTMTVDKSTSTVYMELSSLRSEDTAVYYCARQAFYYFDSWGQGTTVTVSS

SEQ ID NO: 49 - protein, 5 aa, synthetic construct
SYAMS

SEQ ID NO: 50 - protein, 17 aa, synthetic construct
AITFGGGNTYYPDSVKG

SEQ ID NO: 51 - protein, 13 aa, synthetic construct
HGDGNYDFYAMDY

SEQ ID NO: 52 - protein, 17 aa, synthetic construct
KSSQSLLNSGNQKNYLT

SEQ ID NO: 53 - protein, 9 aa, synthetic construct
QNDYSYPLT

SEQ ID NO: 54 - protein, 122 aa, synthetic construct
EVKLVESGGGLVKPGGSLKLSCAASGFTFSSYAMSWVRQTPAKRLEWVAAITFGGGNTYYPDSVKGRFTISRDNARNTLYLQMSSLRSEDTAMYYCARHGDGNYDFYAMDYWGQGTSVTVSS

SEQ ID NO: 55 - protein, 113 aa, synthetic construct
DIVMTQSPSSLTVTAGEKVTMSCKSSQSLLNSGNQKNYLTWYQQKPGQPPKLLIYWASTRESGVPDRFTGSGSRTDFTLTISRVQAEDLAVYYCQNDYSYPLTFGAGTKLELK

SEQ ID NO: 56 - protein, 17 aa, synthetic construct
TISSDGGNTYYTDSVKG

SEQ ID NO: 57 - protein, 9 aa, synthetic construct
HDGRGALDY

SEQ ID NO: 58 - protein, 11 aa, synthetic construct
RASQDISNYLN

SEQ ID NO: 59 - protein, 7 aa, synthetic construct
YTSRLHS

SEQ ID NO: 60 - protein, 9 aa, synthetic construct
QQGNTLPWT

SEQ ID NO: 61 - protein, 118 aa, synthetic construct
EVKLVESGGGLVKPGGSLKLSCAASGFTFSSYAMSWVRQTPAKRLEWVATISSDGGNTYYTDSVKGRFTISRDNARNTLDLQMSSLRSEDTAMYYCARHDGRGALDYWGQGTSVTVSS

SEQ ID NO: 62 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 63 - protein, 5 aa, synthetic construct
NSAVH

SEQ ID NO: 64 - protein, 16 aa, synthetic construct
VIWAGGNTNYNSTLMS

SEQ ID NO: 65 - protein, 10 aa, synthetic construct
HETYGDSFDY

SEQ ID NO: 66 - protein, 16 aa, synthetic construct
RSSQSLLDSDGKTYLN

SEQ ID NO: 67 - protein, 7 aa, synthetic construct
LVSKLDS

SEQ ID NO: 68 - protein, 9 aa, synthetic construct
WQGTHFPFT

SEQ ID NO: 69 - protein, 118 aa, synthetic construct
QVQLKESGPVLVAPSQSLSITCTVSGFSLTNSAVHWVRQPPGKGLEWLGVIWAGGNTNYNSTLMSRLTINKDNSKSQVFLRMNSLQTDDTAIYYCAKHETYGDSFDYWGQGTTLTVSS

SEQ ID NO: 70 - protein, 112 aa, synthetic construct
DVVMTQTPLTLSVTIGQPASISCRSSQSLLDSDGKTYLNWLFQRPGQSPKRLIYLVSKLDSGVPDRFTGSGSGTDFTLKISRVEAEDLGVYYCWQGTHFPFTFGSGTKLEIK

SEQ ID NO: 71 - protein, 259 aa, Homo sapiens
MIPTFTALLCLGLSLGPRTHMQAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWE